(19) United States
(12) Patent Application Publication
(10) Pub. No.: US 2026/0151515 A1
(43) Pub. Date: Jun. 4, 2026

(54) SIGMA-1 COMPOUNDS, RADIOLIGANDS, AND RELATED METHODS OF USE

(71) Applicant: Washington University, St. Louis, MO (US)

(72) Inventors: Zhude Tu, St. Louis, MO (US); Hao Jiang, St. Louis, MO (US); Anil Soda, St. Louis, MO (US); Jiawen Lang, St. Louis, MO (US); Lin Qiu, St. Louis, MO (US)

(73) Assignee: Washington University, St. Louis, MO (US)

(21) Appl. No.: 19/406,923

(22) Filed: Dec. 2, 2025

Related U.S. Application Data
(60) Provisional application No. 63/727,190, filed on Dec. 2, 2024.
(60) Provisional application No. 63/727,192, filed on Dec. 3, 2024.

(51) Int. Cl. A61K 51/04; A61P 25/28; C07D 401/04

(52) U.S. Cl. CPC A61K 51/0455; A61P 25/28; C07D 401/04; A61K 2123/00

(57) ABSTRACT

Among the various aspects of the present disclosure is the provision of sigma-1 compounds, their radioligands, and related methods of use. The present teachings include compositions for compounds that target the sigma-1 receptor, as well as their radioligands. The present teachings also include a method to assess treatment efficacy of a sigma-1 modulator in a subject in need, which can include acquiring medical images after administration of a sigma-1 radioligand, characterizing sigma-1 expression from the acquired images, and assessing treatment efficacy of a sigma-1 modulator in the subject based on the assessed sigma-1 expression. The methods can assess treatment efficacy in neurological diseases, including but not limited to Alzheimer's disease.

### CROSS-REFERENCE TO RELATED APPLICATIONS

This application claims the benefit of priority to U.S. Provisional Application Ser. No. 63/727,190 filed on Dec. 2, 2024, and U.S. Provisional Application Ser. No. 63/727,192 filed on Dec. 3, 2024, which are incorporated herein by reference in its entirety.

### STATEMENT REGARDING FEDERALLY SPONSORED RESEARCH OR DEVELOPMENT

This invention was made with government support under NS075527 and NS103988 awarded by the National Institutes of Health. The government has certain rights in the invention.

### MATERIAL INCORPORATED-BY-REFERENCE

Not applicable.

### FIELD OF THE INVENTION

The present disclosure generally relates to radiolabeled ligands targeting Sigma-1.

### BACKGROUND OF THE INVENTION

The sigma-1 receptor is a unique intracellular protein. It plays a major role in various pathological conditions in the central nervous system (CNS), implicated in several neuropsychiatric disorders. Sigma receptors are recognized as a non-opioid receptor family and have their own specific bioactivity binding patterns; they have a characteristic anatomical expression, unique properties, and modulation functions in the central nervous system. Imaging of sigma-1 receptor in the brain using positron emission tomography (PET) could serve as a noninvasive tool for enhancing the understanding of the disease's pathophysiology. Moreover, σ1R PET tracers can be used for target validation and quantification in diagnosis.

### SUMMARY OF THE INVENTION

Among the various aspects of the present disclosure is the provision of sigma-1 compounds, their radioligands, and related methods of use.

Briefly, therefore, the present disclosure is directed to compositions that target the sigma-1 receptor and can be radiolabeled for imaging purposes to assess sigma-1 expression in vivo, particularly for neurological disorders.

The present teachings include compositions configured to target a sigma-1 receptor in which the composition comprises a compound selected from:

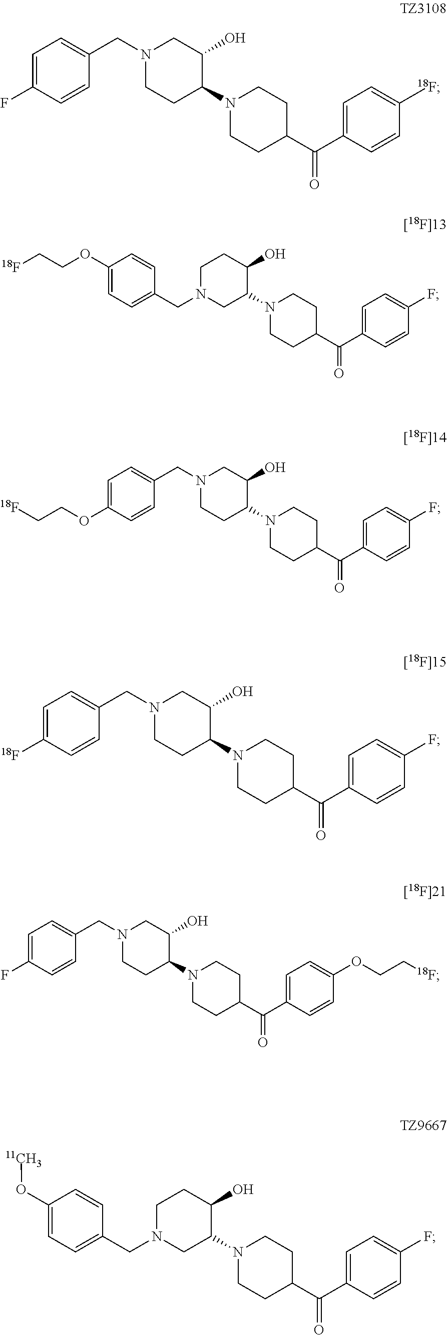

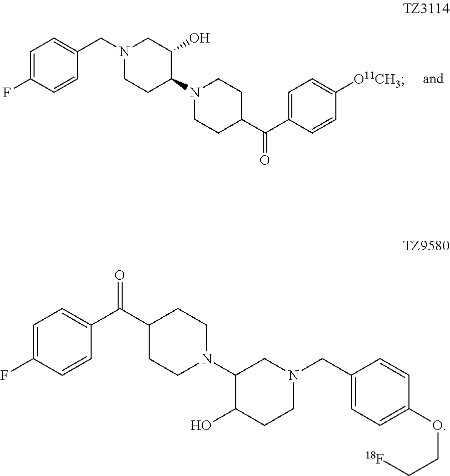

In some aspects, the compound is radiolabeled with a radionuclide. In some aspects, the radionuclide is a radiohalogen. In some aspects, the radiohalogen is F-18. In some aspects, the radionuclide is C-11. In some aspects, the compound is [¹⁸F]13.

The present teachings also include a method to assess treatment efficacy of a sigma-1 modulator in a subject in need. In some aspects, the method can include administering a therapeutically effective amount of a radiolabeled compound configured to target a sigma-1 receptor to the subject; acquiring at least two radioactive images, wherein a first radioactive image is acquired before a sigma-1 modulator treatment begins and a second radioactive image is acquired after the sigma-1 modulator treatment begins; characterizing a sigma-1 expression of the subject based on the acquired images; and assessing a treatment efficacy of a sigma-1 modulator in the subject based on the characterized sigma-1 expression. In some aspects, the radiolabeled compound is configured for uptake to a brain of the subject, specificity for the sigma-1 receptor, and fast metabolic kinetics. In other aspects, the fast metabolic kinetics comprise clearance of the radiolabeled compound from the brain within 60 minutes. In other aspects, the radiolabeled compound is a compound selected from:

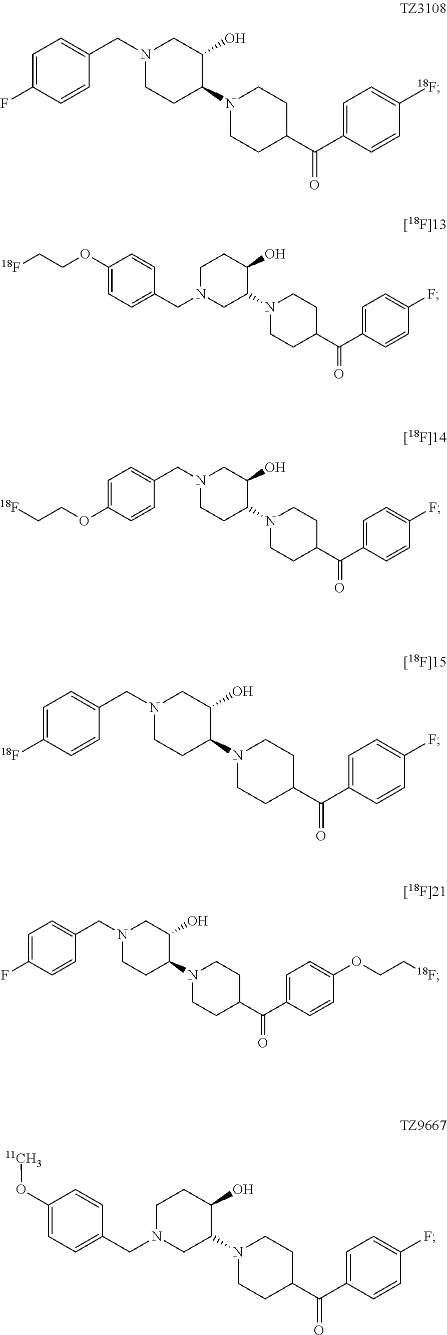

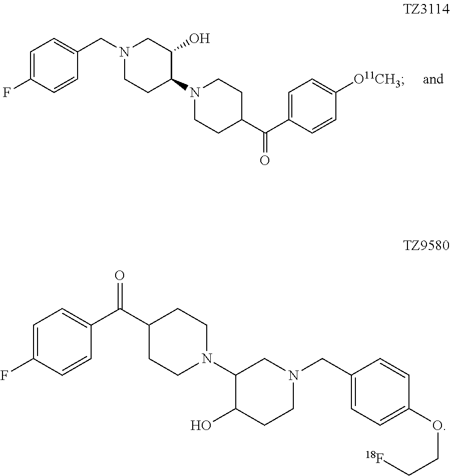

In some aspects, an exemplary embodiment, radiolabeled compound selected is [¹⁸F]13. In other aspects, the at least one radioactive image is selected from PET and SPECT. In other aspects, the radiolabeled compound is administered at a dosage ranging from about 7 MBq to about 370 MBq. In other aspects, the subject has a neurological disorder. In other aspects, the neurological disorder is Alzheimer's disease. In other aspects, assessing the treatment efficacy of the sigma-1 modulator in the subject based on the characterized sigma-1 expression further comprises comparing the characterized expression of sigma-1 receptor in the first radioactive image to the characterized expression of sigma-1 receptor in the second radioactive image, wherein: the treatment is characterized as effective if the characterized expression of sigma-1 receptor in the second radioactive images is the same or increased compared to the characterized expression of sigma-1 receptor in the first radioactive image; and the treatment is characterized as ineffective if the characterized expression of sigma-1 receptor in the second radioactive images is the decreased compared to the characterized expression of sigma-1 receptor in the first radioactive image.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a set of chemical structures of F-18 labeled σ₁R radioligands.

FIG. 1B is a set of graphs showing uptake of the radiotracer (−)-[18F]13 in multiple organs (top) and in the brain (bottom) of SD rats. Ex vivo biodistribution analysis showed a high brain uptake of (−)-[18F]13 with a % ID/g of 0.98±0.11 at 5 min. The uptake was also high in heart, kidney, liver, lung, pancreas, and spleen.

FIG. 1C is a set of images showing in vitro autoradiography analysis that indicated that the uptake of (−)-[¹⁸F]13 was high in the striatum, cortex, thalamus, hippocampus and other gray matter-rich regions of the brain in SD rats, and low in the white matter regions; the distribution of (−)-[¹⁸F]13 matched well with immunohistology of σ₁R in adjacent tissue. Blocking with 10 μM of cold (−)-TZ3108 or Haloperidol can significantly reduce the uptake of (−)-[¹⁸F]13 with nearly no uptake under blocking conditions.

FIG. 1D is a graph of ARG showing that (−)-[¹⁸F]13 had the highest uptake in parietal cortex and striatum and lowest uptake in the corpus callosum and brain stem.

FIG. 1E is a graph showing the relative activity after blocking with 10 μM of cold (−)-[¹⁸F]13 and Haloperidol reduced 55% and 60% of (−)-[¹⁸F]13 uptake with p values of <0.0001 using the student t-test (n=3).

FIG. 2A is a plot and corresponding bar graph showing the standard uptake value (SUV) of σ₁R radioligands in 8-week-old male CD-1 mouse brains (n=3 to 4 per group). At baseline level (blue), radioligands show high brain uptake with a peak SUV of 4.35 at 2 min for (−)-[¹⁸F]13. Preblock with Haloperidol at 2 mg/kg showed a significant reduction of brain uptake for all radioligands (red lines).

FIG. 2B is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 4.35 at 2 min for (−)-[¹⁸F]13. Preblock with cold (−)-TZ3108 at 2 mg/kg showed a significant reduction of brain uptake for all radioligands (green lines).

FIG. 2C is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 4.39 at 3 min for (−)-[¹⁸F]14. Preblock with Haloperidol at 2 mg/kg showed a significant reduction of brain uptake for all radioligands (red lines).

FIG. 2D is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 4.39 at 3 min for (−)-[¹⁸F]14. Preblock with cold (−)-TZ3108 at 2 mg/kg also significantly reduced the brain uptake of all radioligands (green lines).

FIG. 2E is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 3.39 at 2 min for (−)-[¹⁸F]TZ3108. Preblock with Haloperidol at 2 mg/kg showed a significant reduction of brain uptake for all radioligands (red lines).

FIG. 2F is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 3.39 at 2 min for (−)-[¹⁸F]TZ3108. Preblock with cold (−)-TZ3108 at 2 mg/kg also significantly reduced the brain uptake of all radioligands (green lines).

FIG. 2G is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 2.4 at 3 min for (−)-[¹⁸F]15 ((−)-[¹⁸F]TZ3108B). All radioligands washed out from the brain quickly in mouse brains. Preblock with Haloperidol at 2 mg/kg showed a significant reduction of brain uptake for all radioligands (red lines).

FIG. 2H is a plot and corresponding graph showing the standard uptake value (SUV) of σ₁R radioligands in 8-week-old male CD-1 mouse brains (n=3 to 4 per group). At baseline level (blue), radioligands show high brain uptake with a peak SUV of 2.4 at 3 min for (−)-[¹⁸F]15 ((−)-[¹⁸F]TZ3108B). All radioligands washed out from the brain quickly in mouse brains. Preblock with cold (−)-TZ3108 at 2 mg/kg also significantly reduced the brain uptake of all radioligands (green lines).

FIG. 2I is a PET image of a reference brain atlas.

FIG. 2J are representative PET images of (−)-[¹⁸F]13 at baseline, after blocking with Haloperidol, and after blocking with (−)-TZ3108.

FIG. 2K are representative PET images of (−)-[¹⁸F]14 at baseline, after blocking with Haloperidol, and after blocking with (−)-TZ3108.

FIG. 2L are representative PET images of (−)-[¹⁸F]TZ3108 at baseline, after blocking with Haloperidol, and after blocking with (−)-TZ3108.

FIG. 2M are representative PET images of (−)-[¹⁸F]15 at baseline, after blocking with Haloperidol, and after blocking with (−)-TZ3108.

FIG. 3A is a plot of SUV of σ₁R radioligands in 11-month-old female 3xTg-AD and C57BL/6 mouse brains (n=4 to 5 per group). The uptake of (−)-[¹⁸F]13 was significantly lower in the 3xTg-AD mouse brain.

FIG. 3B is a graph of average SUV of σ₁R radioligands in 11-month-old female 3xTg-AD and C57BL/6 mouse brains (n=4 to 5 per group). The uptake of (−)-[¹⁸F]13 was significantly lower in the 3xTg-AD mouse brain.

FIG. 3C is a graph of average SUV of σ₁R radioligands in 11-month-old female 3xTg-AD and C57BL/6 mouse brains (n=4 to 5 per group). The average SUV of (−)-[¹⁸F]13 from 5 to 25 min was 17% lower in 3xTg-AD mice (P=0.0215) and was significantly reduced in all major regions.

FIG. 3D is a plot of SUV of σ₁R radioligands in 11-month-old female 3xTg-AD and C57BL/6 mouse brains (n=4 to 5 per group). The uptake of (−)-[¹⁸F]14 was significantly lower in the 3xTg-AD mouse brain.

FIG. 3E is a graph of average SUV of σ₁R radioligands in 11-month-old female 3xTg-AD and C57BL/6 mouse brains (n=4 to 5 per group). The uptake of (−)-[¹⁸F]14 was significantly lower in the 3xTg-AD mouse brain.

FIG. 3F is a plot of SUV of σ₁R radioligands in 11-month-old female 3xTg-AD and C57BL/6 mouse brains (n=4 to 5 per group). The average SUV of (−)-[¹⁸F]14 from 5 to 25 min was 15.6% lower in 3xTg-AD mice (P=0.0179) and was significantly reduced in major regions.

FIG. 3G is a plot of SUV of σ₁R radioligands in 11-month-old female 3xTg-AD and C57BL/6 mouse brains (n=4 to 5 per group). The uptake of (−)-[¹⁸F]TZ3108 was also significantly lower in the 3xTg-AD mouse brain.

FIG. 3H is a graph of average SUV of σ₁R radioligands in 11-month-old female 3xTg-AD and C57BL/6 mouse brains (n=4 to 5 per group). The uptake of (−)-[¹⁸F]TZ3108 was also significantly lower in the 3xTg-AD mouse brain.

FIG. 3I is a plot of SUV of σ₁R radioligands in 11-month-old female 3xTg-AD and C57BL/6 mouse brains (n=4 to 5 per group). The average SUV of (−)-[¹⁸F]TZ3108 from 5 to 25 min was 18.3% lower in of 3xTg-AD mice (P=0.0341) and was significantly reduced in major regions.

FIG. 3J is a PET image of a reference brain atlas.

FIG. 3K are representative PET images of (−)-[¹⁸F]13 comparing C57BL/6J and 3xTg-AD mice.

FIG. 3L are representative PET images of (−)-[¹⁸F]14 comparing C57BL/6J and 3xTg-AD mice.

FIG. 3M are representative PET images of (−)-[¹⁸F]TZ3108 comparing C57BL/6J and 3xTg-AD mice.

FIG. 4A is a set of immunofluorescent images of σ₁R in the brain of 3xTg-AD (right) and age-matched C57BL/6J mice (left). σ₁R (red) was highly expressed in wild-type C57BL6/J mice whereas a significantly lower number of neuronal σ₁R was identified in the 3xTg-AD mouse brain (Scale bar=50 μm).

FIG. 4B is a graph quantifying the number of σ₁R positive cells per mm² in FIG. 4A. The number of positive neurons in the CA1 regions of the C57BL/6J mouse brain was 2878 per mm² versus 2407 per mm² in 3xTg-AD mouse brain with a p-value of 0.0010 (n=7).

FIG. 4C is a set of immunofluorescent images demonstrating that σ₁R (red) was activated in Aβ plaque-associated astrocytes (white arrows) and low in astrocytes that were distanced from Aβ plaque (pink arrows) (Scale bar=50 μm).

FIG. 5A is a plot of SUV uptake of σ₁R radiotracers in adult male macaque brains. Comparison of brain uptake of the σ₁R radioligands showed (−)-[¹⁸F]TZ3108 had the highest brain uptake with a peak SUV of ˜4.64 at 120 min post-injection, (−)-[¹⁸F]13 and (−)-[¹⁸F]21 had a peak SUV of 3.14 at 27.5 min and ˜2.87 at 105 min post-injection respectively, and (−)-[¹⁸F]14 had a relatively lower SUV.

FIG. 5B is a plot of SUV uptake of σ₁R radiotracers in adult male macaque brains. Comparison of brain uptake of racemic isomers of (−)-[¹⁸F]13. (−)-[¹⁸F]13, (+)-[¹⁸F]13, and (±)-[¹⁸F]13 had high brain uptake but significantly different radiopharmaceutical kinetics, (−)-[¹⁸F]13 showed very good brain washout kinetics whereas (+)-[¹⁸F]13 showed much slower brain washout kinetics post injection.

FIG. 5C is a plot of SUV uptake of σ₁R radiotracers in adult male macaque in specific areas of the brain. Brain regional uptakes of (−)-[¹⁸F]13 in macaque showed that cerebellum had the highest uptake with a peak SUV of 4.12 at 18.5 min post injection, and gray matter regions such as thalamus, frontal cortex, caudate, hippocampus had high uptake, but white matter regions such as corpus callosum had a relatively lower uptake.

FIG. 5D is a set of representative PET images (left) and MRI images (right) of (−)-[¹⁸F]13 distribution in macaque brain, average SUV from 0-120 min in different brain regions in sagittal view.

FIG. 5E is a plot showing radiometabolite analysis of (−)-[¹⁸F]13 in NHP plasma; (−)-[¹⁸F]13 was stable in vivo with 74.79% of total activity remaining as parental (−)-[¹⁸F]13 at 90 min.

FIG. 5F is a representative graph of HPLC-radiometabolite analysis of (−)-[¹⁸F]13 over 20 min.

FIG. 6 contains chemical structure images of the radioligand (−)-[¹⁸F]TZ3108 and other reported σ1R radioligands.

FIG. 7 is a schematic of the synthesis of (±)-13 (or (±)-TZ9580) and (±)-14 (or (±)-TZ96110) along with their enantiomers and hydroxy precursors (±)-11, (−)-11, (+)-11, (±)-12, (−)-12, (+)-12. Reaction and conditions⁸: (a) (C₂H₅)₃N, CH₂Cl₂; (b) m-CPBA, CH₂Cl₂, RT (c) (C₂H₆)₃N, ethanol, 70° C.; (d) acetic anhydride, CH₂Cl₂; (e) 6 N HCl, reflux; (f) BBr₃, CH₂Cl₂, RT, 6 h; (g) 1-Fluoro-2-iodoethane, K₂CO₃, DMF, 120° C., 12 h.

FIG. 8 is a schematic of the synthesis of (±)-TZ3108 and (±)-TZ1064 along with its hydroxy precursor (±)-20. Reaction and conditions^{8,9}: (a) (C₂H₅)₃N, CH₂Cl₂; (b) AcNHOH, K₂CO₃, DMSO, 80° C. (c) 1-Fluoro-2-iodoethane, K₂CO₃, DMF, 120° C., 12 h. The absolute stereochemistry of all ligands was determined by a modified Mosher's method.

FIG. 9 contains ¹H (top) and ¹³C (bottom) NMR spectra of the compound ((3′R,4′R)-1′-(4-(2-fluoroethoxy)benzyl)-4′-hydroxy-[1,3′-bipiperidin]-4-yl)(4-fluorophenyl)methanone [(−)13]((−)-TZ95-80).

FIG. 10 contains ¹H (top) and ¹³C (bottom) NMR spectra of the compound ((3′R,4′R)-1′-(4-(2-fluoroethoxy)benzyl)-3′-hydroxy-[1,4′-bipiperidin]-4-yl)(4-fluorophenyl)methanone [(−)14]((−)-TZ96-110).

FIG. 11 is a schematic of the radiosynthesis of (±)-[¹⁸F]13, (−)-[¹⁸F]13 (+)-[¹⁸F]13, (−)-[¹⁸F]14, (−)-[¹⁸F]15, (−)-[¹⁸F]TZ3-108 and (−)-[¹⁸F]21; Reagents and conditions^{4,7}: (a) [¹⁸F]KF, K₂₂₂, MeCN, 110° C., 10 min; (b) [¹⁸F]18, Cs₂CO₃, DMSO, 110° C., 10 min; (c) CpRu(COD)Cl, ethanol, 85° C., 30 min; (d) Imidazolium chloride (N,N′-bis(2,6-diisopropylphenyl)-2-chloroimidazolium chloride, [¹⁸F]KF, CH₃CN:DMSO (1:1), 160° C., 30 min; (e) [¹⁸F]KF, K₂₂₂, K₂CO₃, TMEDA, DMSO, 150° C., 10 min.

FIG. 12 contains a set of analytical HPLC traces of formulated (−)-[¹⁸F]13, including a UV trace for (−)-[¹⁸F]13 with 10% EtOH in saline (top); (−)-[¹⁸F]13 radio trace (upper center); UV trace for co-injection of (−)-[¹⁸F]13 and cold 13 (lower center); radio trace for co-injection of (−)-[¹⁸F]13 and cold 13 (bottom). Analytical HPLC conditions: Phenomenex Luna SB-C18 column (250×4.6 mm, 5μ), mobile phase 48% acetonitrile in 0.1 M ammonium formate, pH 4.5, flow rate 1.0 mL/min, detection wavelength at 254 nm.

FIG. 13 is a set of analytical HPLC trace of formulated (+)-[¹⁸F]13. UV trace for (+)-[¹⁸F]13 with 10% EtOH in saline (top); (+)-[¹⁸F]13 radio trace (upper center); UV trace for co-injection of (+)-[¹⁸F]13 and cold 13 (lower center); radio trace for co-injection of (+)-[¹⁸F]13 and cold 13 (bottom). Analytical HPLC conditions: Phenomenex Luna SB-C18 column (250×4.6 mm, 5μ), mobile phase 48% acetonitrile in 0.1 M ammonium formate, pH 4.5, flow rate 1.0 mL/min, detection wavelength at 254 nm.

FIG. 14 is a set of analytical HPLC trace of formulated (±)-[¹⁸F]13. UV trace for (±)-[¹⁸F]13 with 10% EtOH in saline (first panel); (±)-[¹⁸F]13 radio trace (second panel); UV trace for co-injection of (±)-[¹⁸F]13 and cold 13 (third panel); radio trace for co-injection of (±)-[¹⁸F]13 and cold 13 (fourth panel). Analytical HPLC conditions: Phenomenex Luna SB-C18 column (250×4.6 mm, 5μ), mobile phase 48% acetonitrile in 0.1 M ammonium formate, pH 4.5, flow rate 1.0 mL/min, detection wavelength at 254 nm.

FIG. 15 is a set of analytical HPLC trace of formulated (−)-[¹⁸F]14. (First panel) UV trace for (−)-[¹⁸F]14 with 10% EtOH in saline; (second panel) (−)-[¹⁸F]14 radio trace; (third panel) UV trace for co-injection of (−)-[¹⁸F]14 and cold 14; (fourth panel) radio trace for co-injection of (−)-[¹⁸F]14 and cold 14. Analytical HPLC conditions: Agilent Zorbax SB-C18 column, 250×4.6 mm, mobile phase 45% acetonitrile in 0.1 M ammonium formate, pH 4.5, flow rate 1.0 mL/min, detection wavelength at 254 nm.

FIG. 16 is a set of analytical HPLC trace of formulated (−)-[¹⁸F]15. (first panel) UV trace for (−)-[¹⁸F]15 with 10% EtOH in saline; (second panel) (−)-[¹⁸F]15 radio trace; (third panel) UV trace for co-injection of (−)-[¹⁸F]15 and cold 15; (fourth panel) radio trace for co-injection of (−)-[¹⁸F]15 and cold 15. Analytical HPLC conditions: Phenomenex Luna SB-C18 column (250×4.6 mm, 5μ), mobile phase 40% acetonitrile in 0.1 M ammonium formate, pH 4.5, flow rate 1.0 mL/min, detection wavelength at 254 nm.

FIG. 17 is a representative HPLC chromatogram for the separation of (−)-[¹⁸F]13. (top) UV absorbance at 254 nm; (bottom) radioactivity detection. The retention time of the product was 21-23 minutes.

FIG. 18 is a representative HPLC chromatogram for the separation of (−)-[¹⁸F]14. (top) UV absorbance at 254 nm; (bottom) radioactivity detection. The retention time of the product was 20-22 minutes.

FIG. 19 is a representative HPLC chromatogram for the separation of (−)-[¹⁸F]15. (top) UV absorbance at 254 nm; (bottom) radioactivity detection. The retention time of the product was 34-36 minutes.

FIG. 20 is a set of representative 3D PET/CT images of (−)-[¹⁸F]13 in normal male CD-1 mice over the course of 60 minutes.

FIG. 21A is a set of immunohistology images of σ1R in mouse brain with a PET image (top left) and magnified regions (right panels). σ1R (red) was heavily expressed in the neuronal cells (green) including cortical and hippocampal neurons with almost all NeuN⁺ cells colocalized with σ1R (scale bar=100 μm).

FIG. 21B is a set of immunohistology images of σ1R in mouse brain with a PET image (top left) and magnified regions (right panels). σ1R (red) was expressed in oligodendrocytes identified by Olig2 (green) with almost all Olig2⁺ cells expressed σ1R (white arrows) (scale bar=100 μm).

FIG. 21C is a set of immunohistology images of σ1R in mouse brain with a PET image (top left) and magnified regions (right panels). σ1R (red) was identified in a portion of astrocyte identified by GFAP (green) staining, while σ1R was not expressed in all GFAP⁺ cells, a high colocalization of σ1R and GFAP at ventricular membrane and neurovascular were identified (white arrows) (scale bar=100 μm).

FIG. 21D is a set of immunohistology images of σ1R in mouse brain with a PET image (top left) and magnified regions (right panels). σ1R (red) was identified in a portion of microglia cells identified by Iba-1 (green) staining (white arrows) (scale bar=100 μm).

FIG. 22A is a plot of radiometabolite analysis of (−)-[¹⁸F]13 in NHP plasma showed (−)-[¹⁸F]13 was stable in vivo with 74.79% of total activity remaining as parental (−)-[¹⁸F]13 at 90 min.

FIG. 22B is a representative image of HPLC-radiometabolite analysis of (−)-[¹⁸F]13 at 60 min.

FIG. 22C is a plot of radiometabolite analysis showing that (+)-[¹⁸F]13 was less stable and metabolized much faster compared to (−)-[¹⁸F]13.

FIG. 22D is a representative image of HPLC-radiometabolite analysis of (+)-[¹⁸F]13 at 60 min.

FIG. 22E is a plot of radiometabolite analysis showing that (−)-[¹⁸F]13 was less stable and metabolized much faster in rat plasma.

FIG. 22F is a representative image of HPLC-radiometabolite analysis of (−)-[¹⁸F]13 at 60 min in rat plasma.

FIG. 22G is a plot of radiometabolite analysis showing that no metabolite of (−)-[¹⁸F]13 could cross BBB and was detectable in rat brain homogenates.

FIG. 22H is a representative image of HPLC-radiometabolite analysis of (−)-[¹⁸F]13 at 60 min in rat brain homogenates.

FIG. 22I is a plot of radiometabolite analysis showing that (−)-[¹⁸F]TZ3108 was less stable compared to (−)-[¹⁸F]13 in vivo with 16.83% of total activity remaining as parental (−)-[¹⁸F]TZ3108 at 60 min.

FIG. 22J is a representative image of HPLC-radiometabolite analysis of (−)-[¹⁸F]TZ3108 at 60 min.

FIG. 22K is a plot of radiometabolite analysis showing that (−)-[¹⁸F]15 has similar in vivo stability to (−)-[¹⁸F]TZ3108.

FIG. 22L is a representative image of HPLC-radiometabolite analysis of (−)-[¹⁸F]15 at 60 min.

FIG. 22M is a plot of radiometabolite analysis showing that (−)-[¹⁸F]TZ3108 was more stable in rat plasma.

FIG. 22N is a representative image of HPLC-radiometabolite analysis of (−)-[¹⁸F]TZ3108 at 60 min in rat plasma.

FIG. 22O is a plot of radiometabolite analysis showing that no metabolite of (−)-[¹⁸F]TZ3108 crossed BBB and was detectable in rat brain homogenates.

FIG. 22P is a representative image of HPLC-radiometabolite analysis of (−)-[¹⁸F]TZ3108 at 60 min in rat brain homogenates.

FIG. 23A is a set of images of (−)-[¹¹C]TZ3114 in NHP brain, including an MRI image (top), an MRI image overlayed on a PET image (middle), and a summed PET image (bottom).

FIG. 23B is a plot of SUV over time showing tissue activity for microPET in the brain.

FIG. 23C is a plot of SUV in specific areas of the brain showing brain tissue time activity curve (TAC) from the baseline scan.

FIG. 24 is a schematic showing the radiosynthesis of (−)[¹¹C]TZ3114 and (+)[¹¹C]TZ3114. Reagents and conditions: (a) [¹¹C]Me-OTf, NaOH, DMF, 90° C., 5 min.

FIG. 25 is a schematic showing the radiosynthesis of (−)-[¹¹C]TZ96-67 and (+)-[¹¹C]TZ96-67. Reagents and conditions: (a) [¹¹C]Me-OTf, NaOH, DMF, 90° C., 5 min.

FIG. 26 is a schematic showing the radiosynthesis of (±)-[¹⁸F]TZ9580 and (−)-[¹⁸F]TZ9580, (+)-[¹⁸F]TZ9580. Reagents and conditions: (a) BBr₃, DCM, 0° C.˜RT; (b) 1-fluoro-2-iodoethane, K₂CO₃, MeCN, 80° C.; (c) [¹⁸F]KF, K₂₂₂, MeCN, 110° C., 10 min; (d) [18F]1, Cs₂CO₃, DMSO, 110° C., 10 min.

FIG. 27 contains a graphs summarizing a competition binding assay of (−)TZ9580 toward σ1R (top), σ2R (middle), and VAChT (bottom). (−)TZ9580 is potent and selective to σ1R with an IC₅₀ of 7.9±1.4 nM (R2=0.91) toward σ1R, an IC₅₀ of 328.3±131.5 nM (R²=0.95) toward σ2R, and an IC₅₀ of 984.7±50.8 nM (R²=0.67) toward VAChT. (+)TZ9580 and the racemic mixture (±)TZ9580 showed a similar potency and selectivity as (−)TZ9580.

FIG. 28 is a set of images showing autoradiography distribution of (−)[¹⁸F]TZ9580 in SD rat brain. Raw autoradiography image (left) and processed color image (middle) showed (−)[¹⁸F]TZ9580 was high in the striatum, cortex, thalamus, hippocampus and other gray matter rich region of the brain, and low in the white matter regions compared to the reference brain atlas (right), immunohistochemistry analysis of σ1R from adjacent tissue matched well with autoradiography signal of (−)[¹⁸F]TZ9580.

FIG. 29 contains images showing autoradiography distribution (−)[¹⁸F]TZ9580 in SD rat brain with blocking. Autoradiography with preblocking with a group of known σ1R compounds showed (−)[18F]TZ9580 is specific to σ1R. Blocking with (−)TZ9580 and the σ1R specific ligand (−)TZ3108 showed a dramatic blockage of (−)[18F]TZ9580 in rat brain. Blocking with haloperidol also showed almost a complete blockage of (−)[18F]TZ9580 signal. In contrast, blocking with PRE-08 and SA4503 showed a slight increase of (−)[18F]TZ9580 uptake.

FIG. 30A is a plot of microPET analysis of (−)[¹⁸F]TZ9580 in mouse brain (n=4). MicroPET analysis was performed in CD1 mouse and the uptake of (−)[¹⁸F]TZ9580 in the brain was high with a peak SUV of 2.68 at 2 min, (−)[¹⁸F]TZ9580 was gradually washed out from the brain over the 60 min of the scan.

FIG. 30B is a plot of microPET analysis of (−)[¹⁸F]TZ9580 in different areas of mouse brain (n=4). The uptake of (−)[¹⁸F]TZ9580 was high in gray matter rich regions such as cortex, striatum, hippocampus, and hypothalamus in consistent with the uptake from autoradiography analysis

FIG. 30C is a representative image of (−)[¹⁸F]TZ9580 in the mouse brain (top) and reference brain atlas “Mouse Brain Benveniste Mirrione” (bottom).

FIG. 31A is a plot showing in vivo microPET blocking of (−)[¹⁸F]TZ9580 in mouse brain (n=3 per group). MicroPET analysis was performed in CD1 mouse and the impact of known σ1R compound on uptake of (−)[¹⁸F]TZ9580 was analyzed. Preblocking with 1 mg/kg of haloperidol 5 min prior to the dose reduced the uptake of (−)[¹⁸F]TZ9580 (red trace).

FIG. 31B is a graph quantifying the average uptake of (−)[¹⁸F]TZ9580 from 20 to 30 min was reduced by 24.4% with a P value of 0.036.

FIG. 31C is a plot showing microPET analysis in CD1 mice and uptake of (−)[¹⁸F]TZ9580. Preblocking with 1 mg/kg of (−)TZ3108 also reduces the uptake of (−)[¹⁸F]TZ9580 (green trace).

FIG. 31D is a graph quantifying the average uptake of (−)[¹⁸F]TZ9580 from 20 to 30 min was reduced by 19.3% with a P value of 0.040.

FIG. 32 is a plot showing the uptake of (−)[¹⁸F]TZ9580 in mouse brain (n=3 per group). (−)[¹⁸F]TZ9580 was induced by pretreatment of 1 mg/kg SA4503 5 min prior to the dose at beginning of the scan (red trace).

FIG. 33A is a plot of SUV of (−)[¹⁸F]TZ9580 (red), (+)[¹⁸F]TZ9580 (green), and (±)[¹⁸F]TZ9580 (blue) in NHP brain. NHP brain PET studies showed the uptake (−)[¹⁸F]TZ9580 was high in the brain with a peak SUV of 3.47 at 15 min, (−)[¹⁸F]TZ9580 the gradually washed out from the brain over 120 min of the scan. In contrast, (+)[¹⁸F]TZ9580 showed a slightly slower and higher uptake than (−)[¹⁸F]TZ9580 with a peak SUV of 3.80 at 100 min, and then slowly washed out. (±)[¹⁸F]TZ9580 showed a slightly higher uptake than (−)[¹⁸F]TZ9580 with a similar dynamic.

FIG. 33B is a plot of SUV of (−)[¹⁸F]TZ9580 in areas of NHP brain. The regional uptake of (−)[¹⁸F]TZ9580 was very high in cerebellum, and also high in other gray matter rich regions including cortex, hippocampus, and thalamus;

FIG. 33C is a plot of SUV of (+)[¹⁸F]TZ9580 in areas of NHP brain. The uptake of (+)[¹⁸F]TZ9580 was also high in different subregions of the brain.

FIG. 34 is a plot of SUV of (−)[¹⁸F]TZ9580 with blocking in NHP brain. Pretreatment with the σ1R compound (−)TZ3108 at 1 mg/kg 5 min prior to the dose showed a significant impact on the uptake of (−)[¹⁸F]TZ9580 (red trace). The uptake was dramatically reduced, the average uptake from 20 to 60 min of (−)[¹⁸F]TZ9580 was reduced by 30% by preblocking with (−)TZ3108.

FIG. 35A is a plot of SUV of (−)[¹⁸F]TZ9580 with blocking in NHP brain. Preblocking with SA4503 induced the uptake of (−)[¹⁸F]TZ9580 (red trace). The increase of (−)[¹⁸F]TZ9580 level possible due to the occupation of σ1R in the peripheral tissues resulted in an enhanced influx of (−)[¹⁸F]TZ9580.

FIG. 35B is a plot of SUV of (−)[¹⁸F]TZ9580 with blocking in NHP brain. Preblocking with different (+)pentazocine induced the uptake of (−)[¹⁸F]TZ9580. The increase of (−)[¹⁸F]TZ9580 level possible due to the occupation of σ1R in the peripheral tissues resulted in an enhanced influx of (−)[¹⁸F]TZ9580.

FIG. 35C is a plot of SUV of (−)[¹⁸F]TZ9580 with blocking in NHP brain. Preblocking with different Yun122 induced the uptake of (−)[¹⁸F]TZ9580. The increase of (−)[¹⁸F]TZ9580 level possible due to the occupation of σ1R in the peripheral tissues resulted in an enhanced influx of (−)[¹⁸F]TZ9580.

FIG. 35D is a plot of SUV of (−)[¹⁸F]TZ9580 with blocking in NHP brain. Preblocking with different haloperidol induced the uptake of (−)[¹⁸F]TZ9580. The increase of (−)[¹⁸F]TZ9580 level possible due to the occupation of σ1R in the peripheral tissues resulted in an enhanced influx of (−)[¹⁸F]TZ9580.

FIG. 36 is a set of plots of NHP plasma radiometabolite analysis of (−)[¹⁸F]TZ9580 (top) and (+)[¹⁸F]TZ9580 (bottom). (−)[¹⁸F]TZ9580 was stable in vivo with a percentage of 72.78% at 90 min post injection, a single peak of radiometabolite was identified with 27.22% of parent compounds 90 min post injection. In contrast, (+)[¹⁸F]TZ9580 metabolized faster than (−)[¹⁸F]TZ9580 with a 32.61% remained parent compound at 90 min post injection and two peaks of metabolite were identified.

FIG. 37 is a plot (left) and corresponding graph (right) of in vivo microPET of (−)[¹⁸F]TZ9580 in 9 month old 3xTg-AD and age-matched C57/Bl6 mouse brain (n=5 per group). The uptake of (−)[¹⁸F]TZ9580 was significantly lower in the 3xTg-AD mice compared to C57/Bl6 WT mice that two-way ANOVA showed F(1, 8)=11.67 with a P value of 0.0091. The average uptake of (−)[¹⁸F]TZ9580 from 10 to 20 min was 15% lower than WT with a p-value of 0.01.

FIG. 38 is a plot (left) and corresponding graph (right) of in vivo microPET of (−)[¹⁸F]TZ9580 in 16 month old 5xFAD and age-matched C57/Bl6 mouse brain (n=3 per group). The uptake of (−)[¹⁸F]TZ9580 was significantly lower in the 5xFAD mice compared to C57/Bl6 WT mice that two-way ANOVA showed (1, 4)=2.51 with a P value of 0.1882; The average uptake of (−)[¹⁸F]TZ9580 from 10 to 20 min was 23.6% lower than WT with a p-value of 0.14.

FIG. 39 is a set of plots showing PET of (−)[¹¹C]TZ3114 in mouse brain. MicroPET analysis was performed in CD1 mouse and the impact of known σ1R compound on uptake of (−)[¹¹C]TZ3114 was analyzed. Preblocking with 2 mg/kg of haloperidol (top) 5 min prior to the dose reduced the uptake of (−)[¹¹C]TZ3114. Preblocking with 2 mg/kg of SA4503 (middle) 5 min prior to the dose induced the uptake of (−)[¹¹C]TZ3114. Preblocking with 2 mg/kg of (−)TZ3108 (bottom) 5 min prior to the dose induced the uptake of (−)[¹¹C]TZ3114.

FIG. 40A is a plot of SUV of (−)[¹¹C]TZ3114 and (+)[¹¹C]TZ3114 in NHP brain. The uptake of (−)[¹¹C]TZ3114 and (+)[¹¹C]TZ3114 was nearly identical with (−)[¹¹C]TZ3114 showed a slightly higher uptake with a peak SUV at 2.43 at 50 min.

FIG. 40B is a plot of SUV of (−)[¹¹C]TZ3114 and (+)[¹¹C]TZ3114 in NHP brain areas. Regional uptake of (−)[¹¹C]TZ3114 was very high in the cerebellum as well as other gray matter rich regions such as cortex, striatum, hippocampus, and thalamus

FIG. 40C is a representative PET image of (−)[¹¹C]TZ3114 in NHP brain.

FIG. 41 is a plot of radiometabolite analysis of (−)[¹¹C]TZ3114 (top) and (+)[¹¹C]TZ3114 (bottom) in NHP plasma samples. Both (−)[¹¹C]TZ3114 and (+)[¹¹C]TZ3114 were stable at the time of tested. No radiometabolite was identified at the time of tested.

FIG. 42A is a plot of SUV of (−)[¹¹C]TZ9667 and (+)[¹¹C]TZ9667 in NHP brain. The uptake of (−)[¹¹C]TZ9667 and (+)[¹¹C]TZ9667 was similar with (+)[¹¹C]TZ9667 showed a slightly higher uptake with a peak SUV at 4.2 at 40 min, the uptake of (−)[¹¹C]TZ9667 had a peak SUV of 3.7 at 20 to 30 min.

FIG. 42B is a plot of SUV of (−)[¹¹C]TZ9667 and (+)[¹¹C]TZ9667 in NHP brain. Regional uptake of (−)[¹¹C]TZ9667 was very high in the cerebellum as well as other gray matter rich regions such as cortex, striatum, hippocampus, and thalamus.

FIG. 42C is a set of representative images of (−)[¹¹C]TZ9667 in NHP brain.

FIG. 43 is a plot of SUV of (±)[¹⁸F]TZ3108 racemic mixture in NHP brain.

FIG. 44 is a plot of SUV of (−)[¹⁸F]TZ3108 in NHP brain. Yun122 and SA4503 affected the tracer dynamics significantly was a bit higher uptake at beginning of the scan and a much faster brain washed out rate and thus a lower uptake at later of the scan. The baseline scan showed a much slower wash out rate with a peak SUV of 5.19 at 45 min of the scan and 4.82 at 120 min

FIG. 45 is a plot of SUV of (−)[¹⁸F]TZ3108 and (+)[¹⁸F]TZ3108 in older NHP brain. All NHPs showed a similar brain uptake and tracer dynamics, no significant trend of wash out was identified over 120 or 180 min of the scan. (−)[¹⁸F]TZ3108 showed a higher uptake than (+)[¹⁸F]TZ3108 on the same subject.

FIG. 46A is a plot of (−)[¹⁸F]TZ3108 in 10 month old 3xTg-AD and age-matched C57/Bl6 mouse brain (n=3 per group). The uptake of (−)[¹⁸F]TZ3108 was significantly lower in the 3xTg-AD mice compared to C57/Bl6 WT mice that two-way ANOVA showed F(1, 4)=19.62 with a P value of 0.0114.

FIG. 46B is a graph of the average uptake of (−)[¹⁸F]TZ3108 from 20 to 40 min was 21.2% lower than WT with a p-value of 0.01.

FIG. 46C is a set of representative images of brain uptake of (−)[¹⁸F]TZ3108 in the C57BL/6 (top) and 3xTg-AD (bottom) mouse brain.

FIG. 47 is a plot (top) and corresponding graph (bottom) of in vivo microPET of (−)[¹⁸F]TZ3108 in 16 month old 5xFAD and age-matched C57/Bl6 mouse brain (n=1 per group). The uptake of (−)[¹⁸F]TZ3108 was significantly lower in the 5xFAD mice compared to C57/Bl6 WT mice. The average uptake of (−)[¹⁸F]TZ3108 from 10 to 20 min was 10% lower than WT.

FIG. 48A contains a set of chemical structures (top) and corresponding graph of SUV uptake in NHP brain. All radiotracers showed good brain uptakes. (±)-[¹⁸F]1, (−)-[¹⁸F]1, (+)-[¹⁸F]1, (−)-[¹⁸F]TZ3108, and (−)-[¹⁸F]3 had a high and (−)-[¹⁸F]2 had the moderate brain uptake in macaque. (−)-[¹⁸F]1 had the initial brain uptake with SUV of ˜2.9 at 30 min and then gradually washout from the brain, suggesting it is the most favorable σ1R radiotracer for brain imaging compared to the other five 18-F σ1R tracers.

FIG. 48B is a graph of SUV of (−)-[¹⁸F]1 in mouse brain showed pretreatment with known σ1R ligand including haloperidol and (±)TZ3108 can significantly reduce the brain uptake compared to the control group indicating (−)-[¹⁸F]1 is specific to σ1R in vivo.

FIG. 48C is a graph of SUV of (−)-[¹⁸F]1 in mouse brain showed the uptake of (−)-[¹⁸F]1 is significantly lower in 3xTg-AD mouse model of AD compared to wildtype control indicating (−)-[¹⁸F]1 is capable of quantifying differential expression of σ1R in AD compared to healthy control.

FIG. 49 is a plot of SUV of (+) and (−) [¹¹C]TZ3-114, both show similar uptake and dynamics. The uptake of (−) [¹¹C]TZ3-114 is slightly lower than (+) [¹¹C]TZ3-114.

FIG. 50A is a plot of SUV of (+) and (−) [¹¹C]TZ96-67. Both show similar uptake and dynamics. Blocking with cold (−)TZ96-67 showed a decrease of brain uptake of (−) [¹¹C]TZ96-67.

FIG. 50B is a plot of SUV of (−)-[¹¹C]TZ96-67. Blocking with cold (−)TZ96-67 showed a decrease of brain uptake of (−) [¹¹C]TZ96-67.

FIG. 51 is a plot of SUV of (±) and (−) [¹⁸F]TZ95-80 showed different uptakes. (±)-[¹⁸F]TZ95-80 showed higher uptake and clearly trend of washout, whereas (−)-[¹⁸F]TZ95-80 showed lower uptake but similar trend of washout in the same monkey. Lou. (−)-[¹⁸F]TZ95-80 showed similar uptake and dynamics on Ollie.

FIG. 52 is a plot of SUV of (−) [¹⁸F]TZ95-80 was done with SA4503 at 1.5 mg/kg and Yun122 at 1 mg/kg 5 min before injection of tracer. Displacement study was done with SA4503 at 1.5 mg/kg 20 post injection.

FIG. 53 contains a set of chemical structures (bottom) and corresponding SUV plot (top) of (−)[¹⁸F]TZ3-108B, which shows similar tracer dynamics as (−)[¹⁸F]TZ3-108.

FIG. 54 contains a set of plots showing the SUV of (+)[¹¹C]TZ96-105 (top) and (−)[¹¹C]TZ96-105 (bottom), which show identical tracer dynamics. Both entered the brain quickly and gradually increased without a trend of washing out.

FIG. 55 is a plot showing the SUV of (−)[¹⁸F]TZ96-110 entered the brain quickly and gradually increased without a trend of washing out.

FIG. 56 contains a chemical structure and a set of plots showing radiotracer uptake of (−)[¹⁸F]TZ3-108 (top) and (−)[¹⁸F]TZ3-108B (bottom). In vivo blocking of (+) and (−) [18F]TZ3-108 with SA4503, (±)TZ3-108, and Haloperidol.

FIG. 57 contains a chemical structure and a set of plots showing radiotracer uptake of (−)TZ96-105. In vivo blocking of (−)[¹¹C]TZ96-105 with SA4503, (±)TZ3-108, and Haloperidol.

FIG. 58 contains a chemical structure and a set of plots showing radiotracer uptake of (−)-TZ96-110. In vivo blocking of (−)[¹⁸F]TZ96-110 with SA4503, (±)TZ3-108, and Haloperidol.

FIG. 59 contains a chemical structure and a set of plots showing radiotracer uptake of (−)-TZ96-110. In vivo microPET study of (−)[¹⁸F]TZ96110 in 10 month old 3xTg-AD and age-matched C57/Bl6 mouse brain (n=3 per group).

### DETAILED DESCRIPTION OF THE INVENTION

The present disclosure is based, at least in part, on the development of new molecules targeting on sigma-1 protein. In vitro binding studies demonstrated these compounds are potent and selective for sigma-1 receptor. C-11 and F-18 radiochemistry of making these radiotracers was further performed and in vitro and in vivo binding properties were characterized. The data identified a promising Sigma-1 receptor PET radiotracer for imaging Sigma-1 for neurological diseases and/or psychiatric abnormalities. This exemplary embodiment may be used for assessing the therapeutical efficacy of sigma-1 modulator(s) for treating AD diseases.

As shown herein, radiosynthesis and in vivo evaluation of Carbon-11 PET ligands for imaging the σ1 receptors in the brain and radiosynthesis and in vivo evaluation of six F-18 radioligands for imaging sigma-1 receptor in the brain are described.

One aspect of the present disclosure provides radiolabeled compounds for imaging of sigma-1. In some aspects, the compounds can be radiolabeled with C-11. In other aspects, the compounds can be radiolabeled with F-18.

In one aspect of the present disclosure, the σ1R radioligands may comprise any one of the structures of Table 1.

TABLE 1
σ1R radioligands

| Compound | |
|---|---|
| Name | Structure |
| TZ3108 | 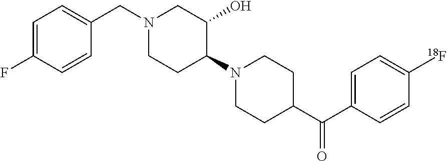 [SMILES: O=C(c1ccc([18F])cc1)C1CCN([C@H]2CCN(Cc3ccc(F)cc3)C[C@@H]2O)CC1] |
| [¹⁸F]13 | 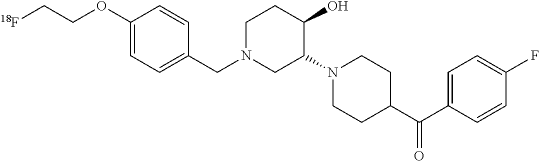 |
| [¹⁸F]14 | 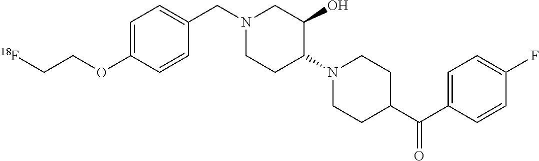 |
| [¹⁸F]15 | 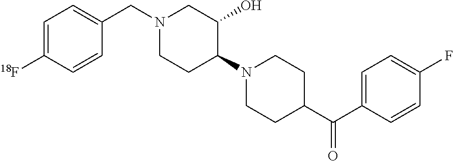 [SMILES: O=C(c1ccc(F)cc1)C1CCN([C@H]2CCN(Cc3ccc([18F])cc3)C[C@@H]2O)CC1] |
| [¹⁸F]21 | 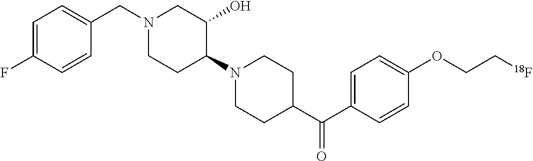 [SMILES: O=C(c1ccc(OCC[18F])cc1)C1CCN([C@H]2CCN(Cc3ccc(F)cc3)C[C@@H]2O)CC1] |
| TZ9667 | 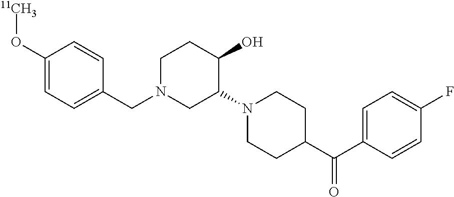 |
| TZ3114 | 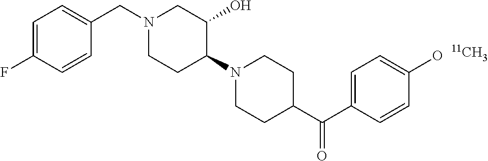 |
| TZ9580 | 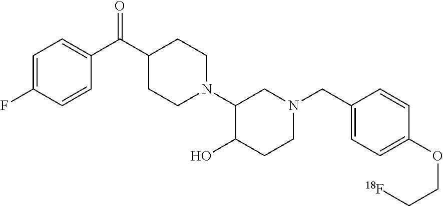 [SMILES: O=C(c1ccc(F)cc1)C1CCN(C2CN(Cc3ccc(OCC[18F])cc3)CCC2O)CC1] |

### Sigma-1 Modulation Agents

As described herein, sigma-1 expression has been implicated in various diseases, disorders, and conditions. As such, modulation of sigma-1 (e.g., modulation of sigma-1 receptors in an Alzheimer's disease patient) can be used for treatment of such conditions. A sigma-1 modulation agent can modulate sigma-1 response or induce or inhibit sigma-1. Sigma-1 modulation can comprise modulating the expression of sigma-1 on cells, modulating the quantity of cells that express sigma-1, or modulating the quality of the sigma-1-expressing cells.

Sigma-1 modulation agents can be any composition or method that can modulate sigma-1 expression on cells (e.g., a small molecule that agonizes sigma-1). For example, a sigma-1 modulation agent can be an activator, an inhibitor, an agonist, or an antagonist. As another example, the sigma-1 modulation can be the result of gene editing.

A sigma-1 modulation agent can be an anti-sigma-1 antibody (e.g., a monoclonal antibody to sigma-1).

A sigma-1 modulating agent can be an agent that induces or inhibits progenitor cell differentiation into sigma-1 expressing cells. For example, a small molecule can be used to agonize sigma-1.

Sigma-1 Signal Modulation by Small Molecule Inhibitors, shRNA, siRNA, or ASOs

As described herein, a sigma-1 modulation agent can be used for use in therapy for a neurodegenerative disease, including but not limited to Alzheimer's disease. A sigma-1 modulation agent can be used to reduce/eliminate or enhance/increase sigma-1 signals. For example, a sigma-1 modulation agent can be a small molecule agonizer of sigma-1. As another example, a sigma-1 modulation agent can be a short hairpin RNA (shRNA). As another example, a sigma-1 modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Sigma-1 Modulating Agent

One aspect of the present disclosure provides for targeting of sigma-1, its receptor, or its downstream signaling. The present disclosure provides methods of treating or preventing a neurodegenerative disease based on the discovery that imaging sigma-1 in a subject can be used to assess therapeutic efficacy in a neurodegenerative disease of the subject.

As described herein, modulators of sigma-1 (e.g., antibodies, fusion proteins, small molecules) can reduce or prevent a neurodegenerative disease, including but not limited to Alzheimer's disease. A sigma-1 modulation agent can be any agent that can enhance sigma-1, upregulate sigma-1, or knock-in sigma-1.

As an example, a sigma-1 modulation agent can modulate sigma-1 signaling.

For example, the sigma-1 modulating agent can be an anti-sigma-1 antibody. Furthermore, the anti-sigma-1 antibody can be a murine antibody, a humanized murine antibody, or a human antibody.

As another example, the sigma-1 modulating agent can be an anti-sigma-1 antibody, wherein the anti-sigma1 antibody prevents binding of sigma-1 to its receptor or prevents activation of sigma-1 and downstream signaling.

As another example, the sigma-1 modulating agent can be a fusion protein. For example, the fusion protein can be a decoy receptor for sigma-1. Furthermore, the fusion protein can comprise a mouse or human Fc antibody domain fused to the ectodomain of sigma-1.

As another example, a sigma-1 modulating agent can be an inhibitory protein that antagonizes sigma-1. In another example, a sigma-1 modulating agent can be an activating protein that agonizes sigma-1. For example, the sigma-1 modulating agent can be a viral inhibitory protein that antagonizes sigma-1, which has been shown to antagonize sigma-1. In another example, the sigma-1 modulating agent can be a viral activating protein that agonizes sigma-1, which has been shown to agonize sigma-1.

As another example, a sigma-1 modulating agent can be a short hairpin RNA (shRNA) or a short interfering RNA (siRNA) targeting sigma-1 or associated pathways.

As another example, a sigma-1 modulating agent can be an sgRNA targeting sigma-1 or associated pathways.

Methods for preparing a sigma-1 modulating agent (e.g., an agent capable of modulating sigma-1 signaling) can comprise construction of a protein/Ab scaffold containing the natural sigma-1 receptor as a sigma-1 neutralizing agent; developing modulators of the sigma-1 receptor “down-stream”; or developing modulators of the sigma-1 production “up-stream”.

Modulating sigma-1 can be performed by genetically modifying sigma-1 in a subject or genetically modifying a subject to modulate expression of the sigma-1 gene, such as through the use of CRISPR-Cas9 or analogous technologies, wherein, such modification reduces or prevents a neurodegenerative disease.

### Chemical Agent:

Examples of sigma-1 imaging agents are described herein. In some aspects, the sigma-1 imaging agents are _{(±)-}TZ3-108, ₍₋₎₋TZ3-108, or ₍₊₎₋TZ3-108, as shown below:

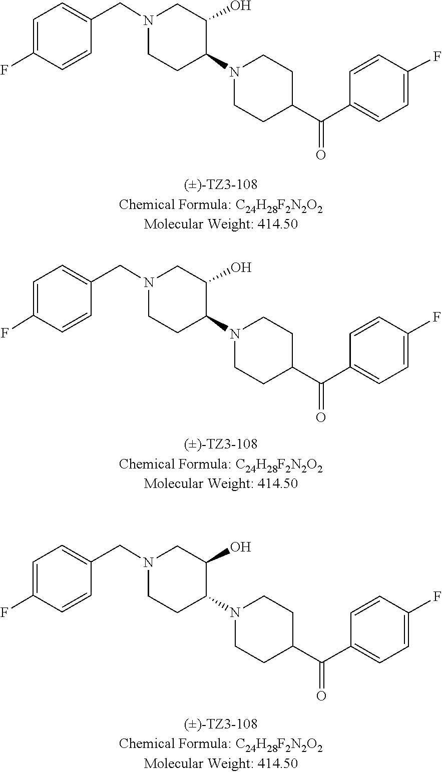 [SMILES: O=C(c1ccc(F)cc1)C1CCN([C@H]2CCN(Cc3ccc(F)cc3)C[C@@H]2O)CC1 ; O=C(c1ccc(F)cc1)C1CCN([C@H]2CCN(Cc3ccc(F)cc3)C[C@@H]2O)CC1 ; O=C(c1ccc(F)cc1)C1CCN([C@@H]2CCN(Cc3ccc(F)cc3)C[C@H]2O)CC1]

In other embodiments, the sigma-1 imaging agents can be any compound comprising the structures of Table 2 below:

TABLE 2
Sigma-1 Imaging Agents

| Compound | |
|---|---|
| Name | Structure |
| TZ3-108 | 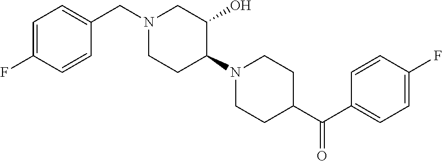 [SMILES: O=C(c1ccc(F)cc1)C1CCN([C@H]2CCN(Cc3ccc(F)cc3)C[C@@H]2O)CC1] |
| TZ96-105 | 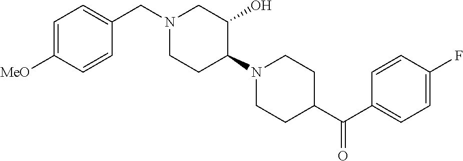 [SMILES: COc1ccc(CN2CC[C@H](N3CCC(C(=O)c4ccc(F)cc4)CC3)[C@@H](O)C2)cc1] |
| TZ3-114 | 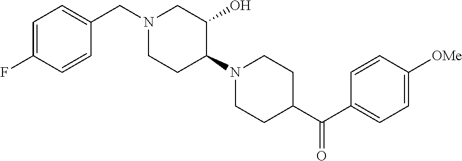 [SMILES: COc1ccc(C(=O)C2CCN([C@H]3CCN(Cc4ccc(F)cc4)C[C@@H]3O)CC2)cc1] |
| TZ96-110 | 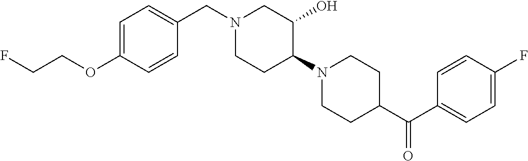 [SMILES: O=C(c1ccc(F)cc1)C1CCN([C@H]2CCN(Cc3ccc(OCCF)cc3)C[C@@H]2O)CC1] |
| TZ96-67 | 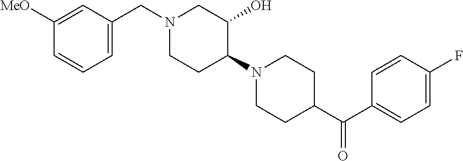 [SMILES: COc1cccc(CN2CC[C@H](N3CCC(C(=O)c4ccc(F)cc4)CC3)[C@@H](O)C2)c1] |
| TZ95-80 | 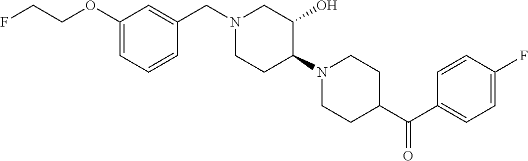 [SMILES: O=C(c1ccc(F)cc1)C1CCN([C@H]2CCN(Cc3cccc(OCCF)c3)C[C@@H]2O)CC1] |

In other aspects, the F of the chemical agents shown above comprise F-18 radiolabels. In other aspects, the above compound can be radiolabeled with C-11.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O— CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Genome Editing

As described herein, sigma-1 signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate modulation of sigma-1 by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for neurodegenerative diseases to target cells by the modulation of sigma-1 signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a neurodegenerative disease in a subject in need of administration of a therapeutically effective amount of a sigma-1 modulation agent, as assessed by the sigma-1 imaging methods of the present disclosure, so as to treat a neurodegenerative disease.

Methods described herein are generally performed on a subject in need thereof. A subject in need of the therapeutic methods described herein can be a subject having, diagnosed with, suspected of having, or at risk for developing a neurodegenerative disease, including but not limited to Alzheimer's disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a sigma-1 modulation agent is, for example, an amount that would cause the desired therapeutic effect in a subject while minimizing undesired side effects. In various embodiments, an effective amount of a sigma-1 modulation agent described herein can substantially inhibit a neurodegenerative disease, slow the progress of a neurodegenerative disease, or limit the development of a neurodegenerative disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of the radiolabeled imaging agent of the present disclosure or subsequent therapeutic compounds administered such as a sigma-1 modulator can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a neurodegenerative disease.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the radiolabeled imaging agents of the present disclosure or subsequent therapeutic administrations can occur as a single event or over a time course of treatment. For example, the agents can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a neurodegenerative disease.

A sigma-1 agent, including the imaging agents of the present disclosure and sigma-1 modulation agents can be administered simultaneously or sequentially with another agent, such as an antibiotic, an anti-inflammatory, or another agent. For example, a sigma-1 agent can be administered simultaneously with another agent, such as an antibiotic or an anti-inflammatory. Simultaneous administration can occur through administration of separate compositions, each containing one or more of a sigma-1 modulation agent, an antibiotic, an anti-inflammatory, or another agent. Simultaneous administration can occur through administration of one composition containing two or more of a sigma-1 modulation agent, an antibiotic, an anti-inflammatory, or another agent. A sigma-1 agent can be administered sequentially with an antibiotic, an anti-inflammatory, or another agent. For example, a sigma-1 agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to, the radiolabeled sigma-1 compounds of the present disclosure, solvents, solubilizers, syringes, and sterile packaging. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

The examples describe the synthetic methods of synthesizing radiolabeled ligands for sigma-1, characterizing their binding performance in vitro and in vivo, and performing PET imaging experiments with the ligands.

### Example 1—Design, Synthesis, and Characterization of F-18 Sigma-1 Receptor Radiotracers for Alzheimer Disease

### Background

Alzheimer's disease (AD) is the most common form of dementia with nearly 7 million Americans living with AD. Previous studies reported the importance and significance of the sigma-1 receptor (σ₁R) for its neuroprotective effects in neurological disorders. Mounting evidence highlights the association of σ₁R with AD pathologies. Pioneer studies in postmortem AD tissues using sigma receptor agonists [³H]DTG found a 26% reduction of sigma receptors in the hippocampus. PET imaging with σ₁R radioligand [¹¹C]SA4503 in early AD patients showed a low σ₁R density in cerebral and cerebellar regions and an increase of σ₁R in various brain regions but a decrease in the hippocampus, suggesting elevated σ₁R expression in early AD and a decrease as the disease progresses. Mechanistic studies using an σ₁R agonist, N,N-Dimethyltryptamine (DMT), demonstrated decreased σ₁R expression and disrupted neuronal endoplasmic reticulum (ER)-mitochondria signaling in AD. These findings unveil the critical role of σ₁R in AD pathogenesis and its great potential for early AD monitoring, progression tracking, and therapeutic intervention.

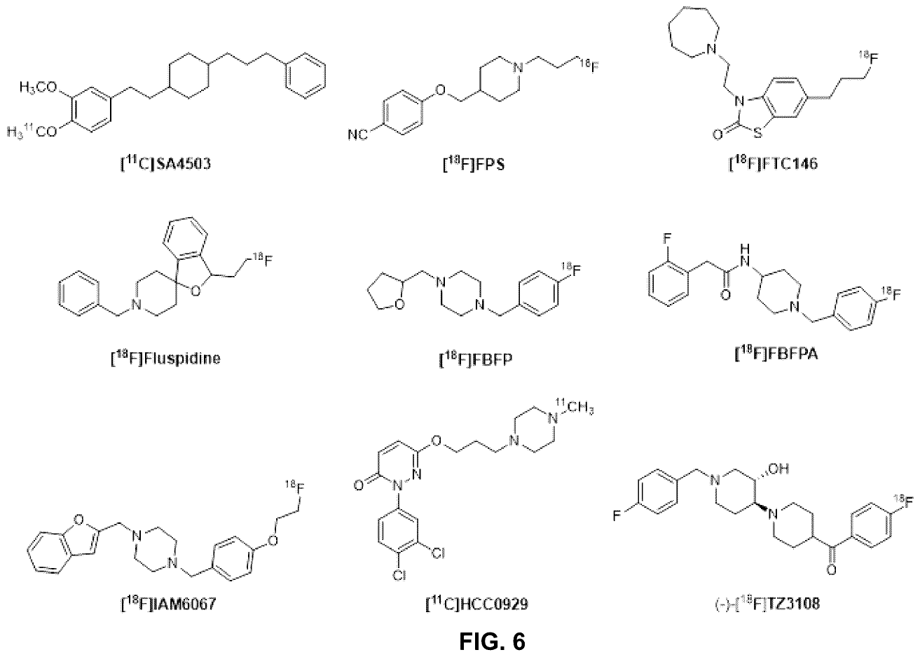

Non-invasive PET with a σ₁R-specific radioligand could serve as a powerful tool for in vivo assessment of σ₁R expression. Several radioligands have been developed to quantify σ₁R expression in the brain with a few tested for clinical use (FIG. 6). [¹¹C]SA4503 was the first radioligand for human studies showing differential expression of σ₁R in AD. However, its low selectivity limited its use and led to inconsistent results across studies. [¹⁸F]FPS exhibited slow dynamics and struggled to reach transient equilibrium in four hours due to high affinity, limiting its application. [¹⁸F]FTC-146, also faced challenges in reaching equilibrium within three hours. Other radioligands have various limitations, including low selectivity, irreversible binding profiles, slow dynamics, or low in vivo specificity, hindering their progression to clinical studies.

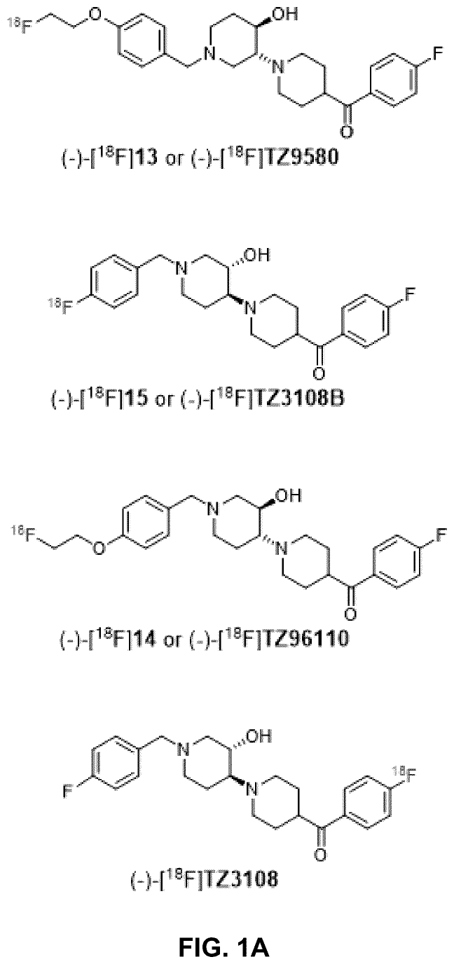

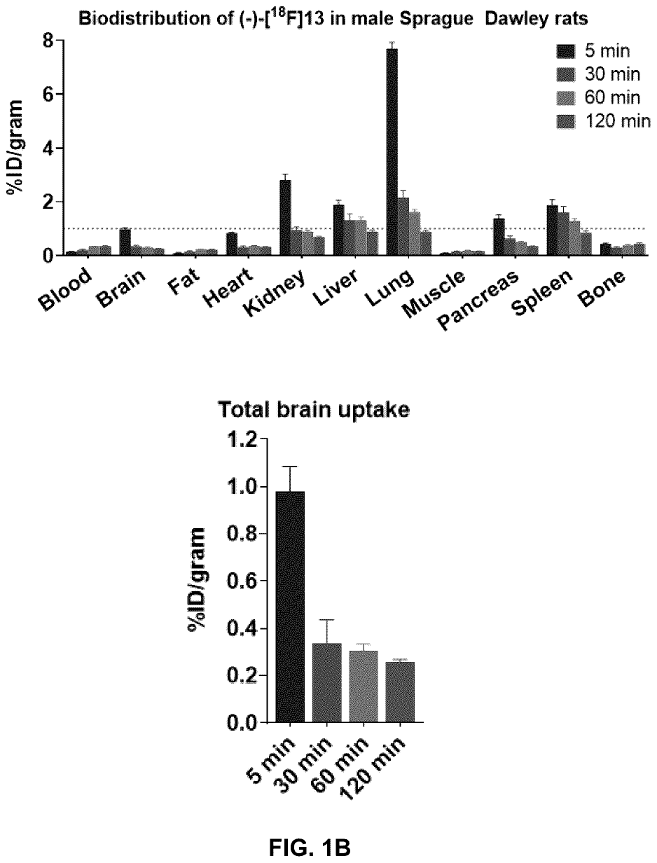

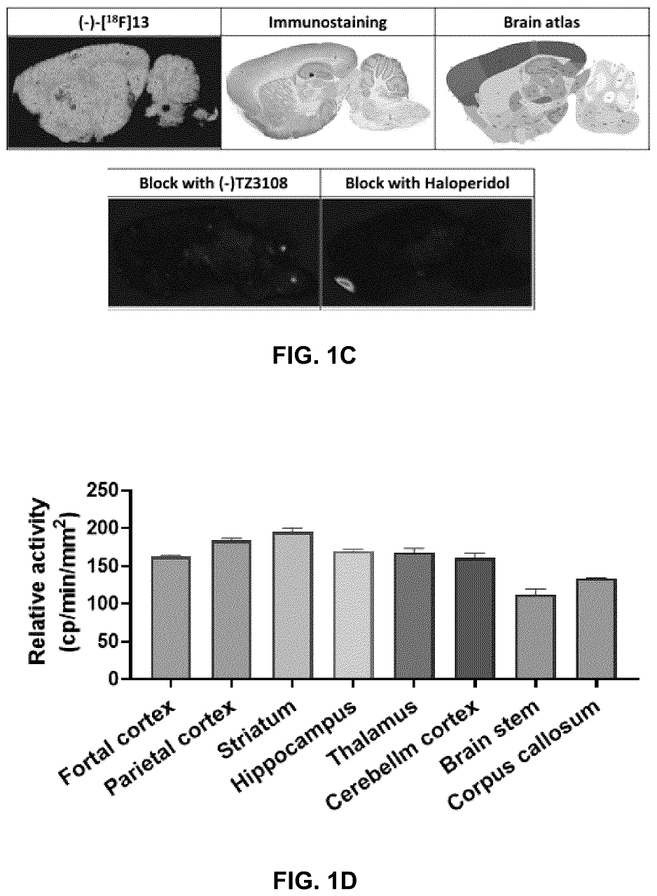

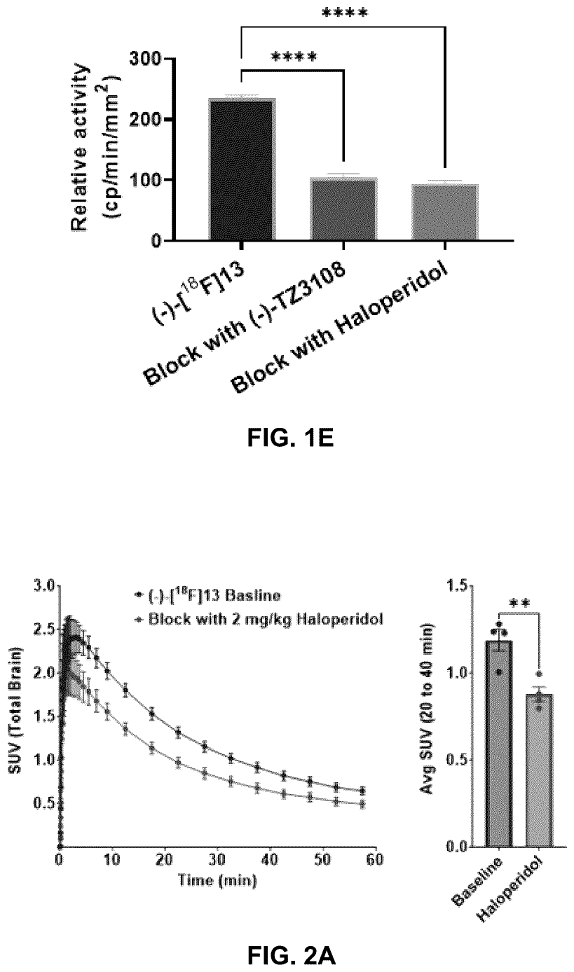

A previously synthesized σ₁R ligand, (−)-TZ3108 (FIG. 6), is highly potent and selective to σ₁R and has high brain uptake in animals. PET studies in nonhuman primates (NHP) showed (−)-[¹⁸F]TZ3108 achieved equilibrium ˜45 min post-injection and can be blocked with known σ₁R compounds in vivo. To further understand the molecular properties of (−)-[¹⁸F]TZ3108 and to develop a promising F-18 labeled σ₁R radioligand for clinical usage, in this study, the in vivo properties of (−)-[¹⁸F]TZ3108 in AD mice are characterized. Additionally, an alternative F-18 methodology was explored to introduce F-18 into the fluorobenzene for (−)-[¹⁸F]15 (also named (−)-[¹⁸F]TZ3108B) (FIG. 1A) to test if the differential labeling position could improve brain washout pharmacokinetics. Also designed and synthesized were three new radiotracers (−)-[¹⁸F]13, (−)-[¹⁸F]14, and (−)-[¹⁸F]21 and performed a systematic characterization of these radioligands in rodents and NHPs. PET studies with the leading radioligands in the 3xTg-AD mice demonstrated all radioligands could detect the reduced σ₁R expression in AD. The results indicated (−)-[¹⁸F]13 is a promising σ₁R radioligand and worth seeking FDA approval for further clinical investigations in AD and other neurodegenerative diseases.

### Results

### Synthesis of σ₁R Ligands

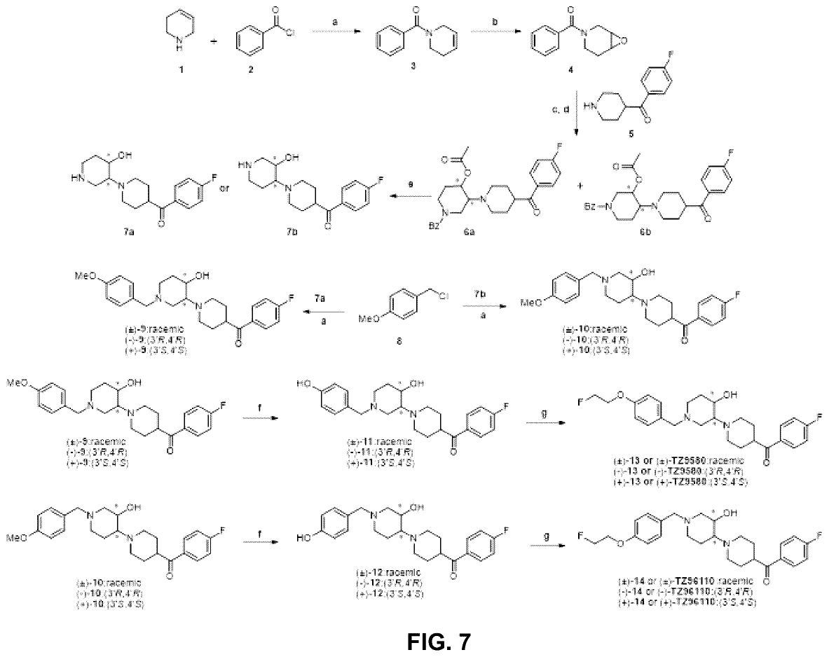

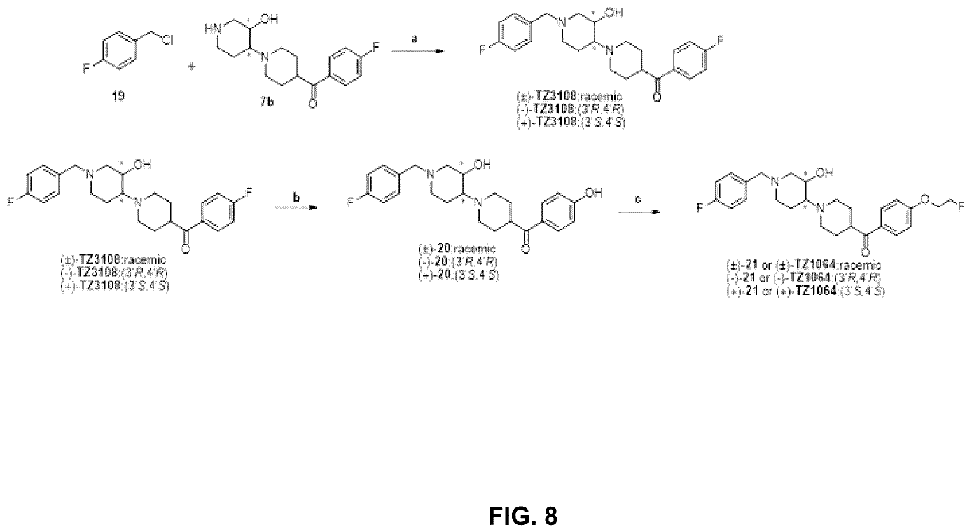

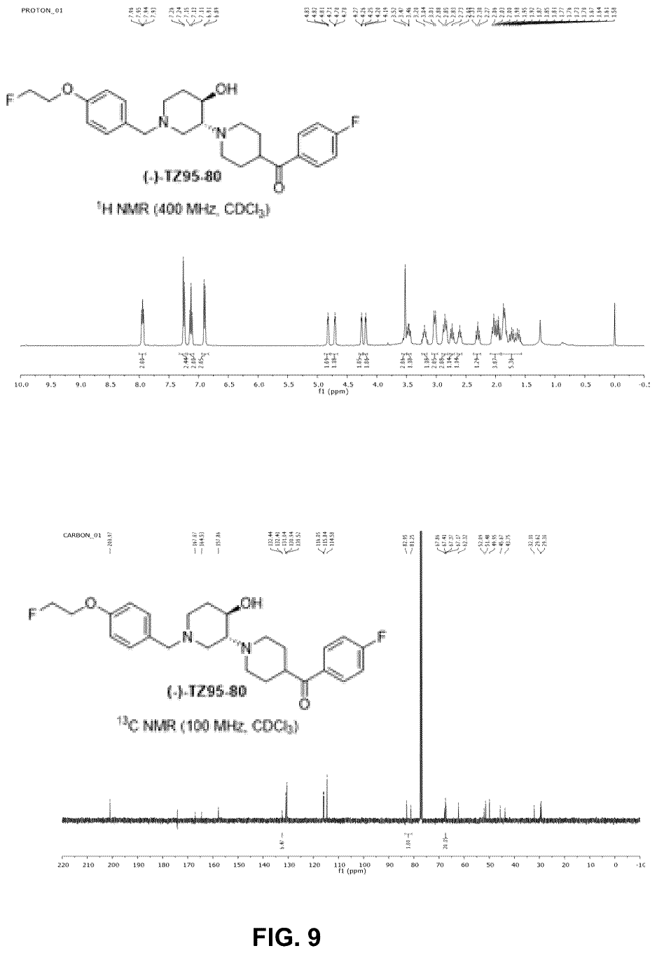

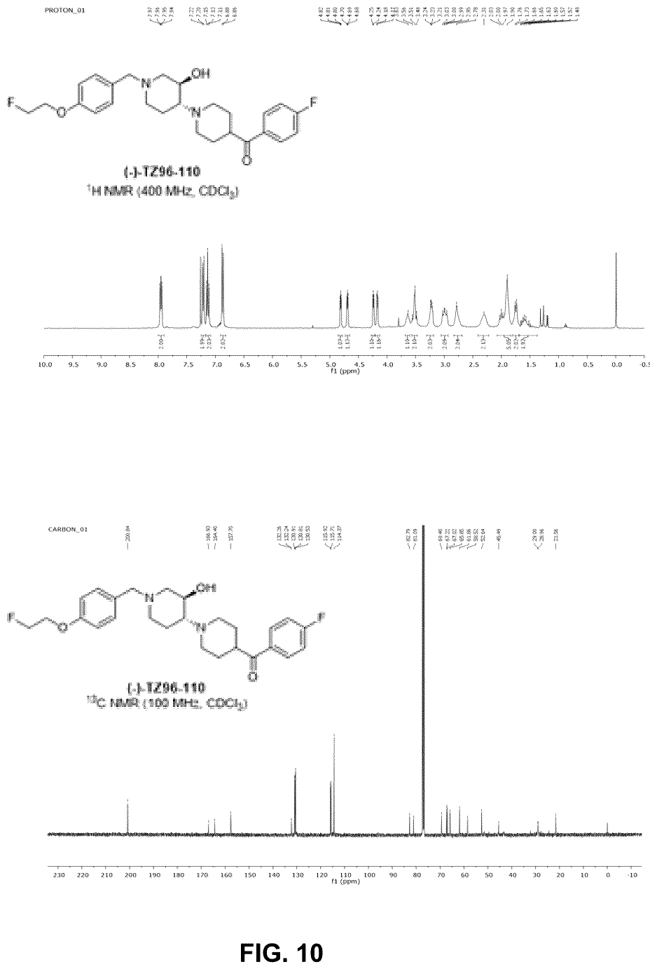

Racemic σ₁R compounds, (±)-13, (±)-14, (±)-15, and (±)-21 along with their enantiomers, and hydroxy precursors (±)-11 for (±)-[¹⁸F]13, (−)-11 for (−)-[¹⁸F]13, (+)-11 for (+)-[¹⁸F]13, (−)-12 for (−)-14 and (−)-15, (−)-20 for (−)-21 were synthesized according to the reported procedures (FIG. 7, FIG. 8). The absolute configuration and NMR data of these molecules, providing structural evidence, have previously been confirmed. The HPLC mobile phase and column details for the unknown compounds, along with the enantiomeric excess (ee) of chiral isomers determined by normal phase HPLC (FIG. 9, FIG. 10).

### In Vitro Characterization of Potency and Specificity of Novel σ₁R Ligands

All compounds, including racemic mixture, (−) and (+) isoforms of compounds 13, 14, and 21 were highly potent and selective to σ₁R over σ₂R and VAChT proteins (Table 3). For example, (±)-13, (−)-13, and (+)-13 had Ki of values 6.1±1.5, 7.7±1.4, and 2.7±0.9 nM to σ₁R. These compounds were also highly selective to σ₁R over σ₂R and VAChT, for example, (−)-13 was 79.3-fold selective to σ₁R over σ₂R and 93.6-fold selective to σ₁R over VAChT. Compound 15 shares the same structure as TZ3108. Overall, the change of fluorobenzyl on either side of TZ3108 to fluoroethoxy benzyl on compounds 14 or 21 reduced the potency and selectivity of these compounds, indicating a key role of fluorobenzyl from both sides of TZ3108 on its molecular interaction with σ₁R protein. Whereas changing 1,4′-bipiperidin on 14 to 1,3′-bipiperidin on 13 showed no significant impact. Overall, all newly synthesized ligands are potent and selective to σ₁R and are suitable for further characterization in vivo.

TABLE 3
Binding affinities of candidate compounds σ₁R, σ₂R, and VAChT.

| | Ki (nM) | | | Selective Ratio | |
|---|---|---|---|---|---|
| Compounds | σ₁R | σ₂R | VAChT | σ₁R/σ₂R | σ₁R/VAChT |
| (±)-13 | 6.1 ± 1.5 | 587.9 ± 51.8 | 626.3 ± 53.1 | 96.4 | 102.7 |
| (−)-13 | 7.7 ± 1.4 | 610.8 ± 32.6 | 720.6 ± 34.0 | 79.3 | 93.6 |
| (+)-13 | 2.7 ± 0.9 | 563.6 ± 41.2 | 536.1 ± 19.9 | 208.7 | 198.6 |
| (±)-14 | 7.8 ± 0.4 | 738.5 ± 37.4 | 727.8 ± 21.8 | 94.7 | 93.3 |
| (−)-14 | 9.3 ± 1.5 | 926.0 ± 70.1 | 961.6 ± 127.2 | 99.6 | 103.4 |
| (+)-14 | 6.6 ± 1.6 | 809.6 ± 52.4 | 564.0 ± 15.1 | 122.7 | 85.5 |
| (±)-21 | 15.2 ± 2.1 | 641.7 ± 16.9 | 487.2 ± 49.9 | 42.2 | 32.1 |
| (−)-21 | 15.4 ± 3.9 | 511.2 ± 13.0 | 541.7 ± 23.9 | 33.2 | 35.2 |
| (+)-21 | 6.8 ± 0.4 | 556.4 ± 29.5 | 673.4 ± 24.4 | 81.8 | 99.0 |
| (±)-TZ3108* | 0.48 ± 0.14 | 1740 ± 280 | 1360 ± 295 | 3625.0 | 2833.3 |
| (−)-TZ3108* | 1.8 ± 0.4 | 6960 ± 810 | 980 ± 87 | 3866.7 | 544.4 |
| (+)-TZ3108* | 0.14 ± 0.02 | 2390 ± 340 | 1090 ± 200 | 17071.4 | 7785.7 |

### Radiosynthesis of Novel F-18 σ₁R Tracers

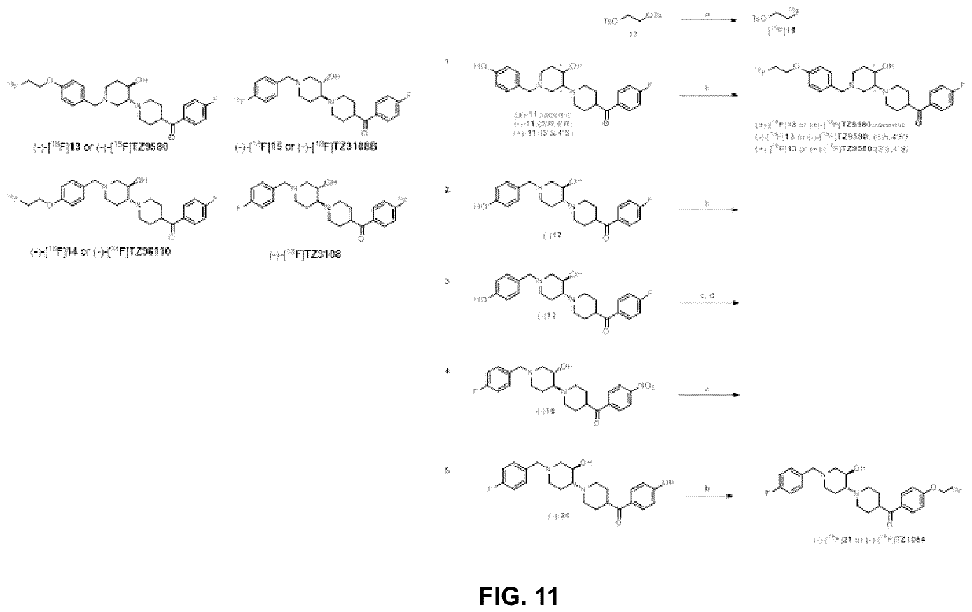

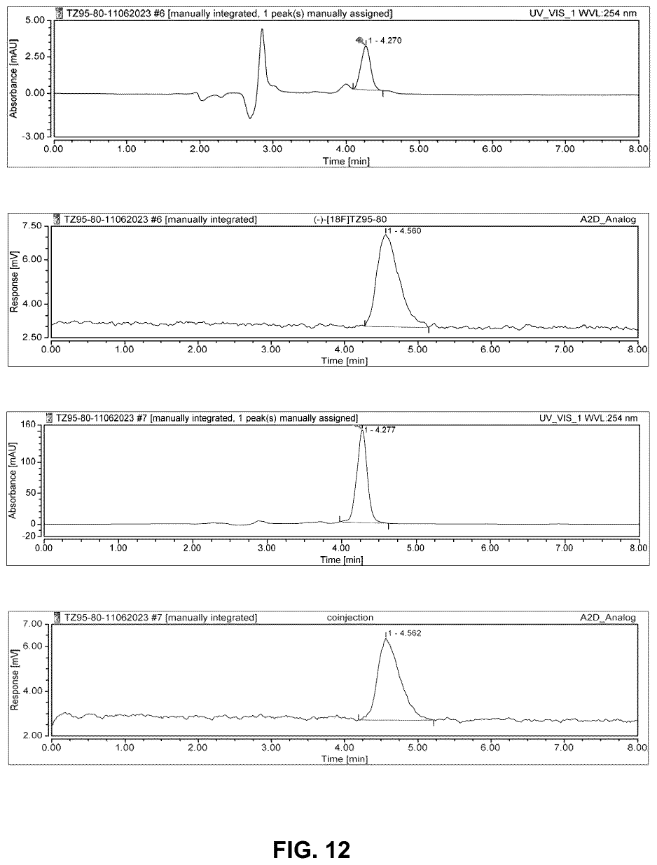

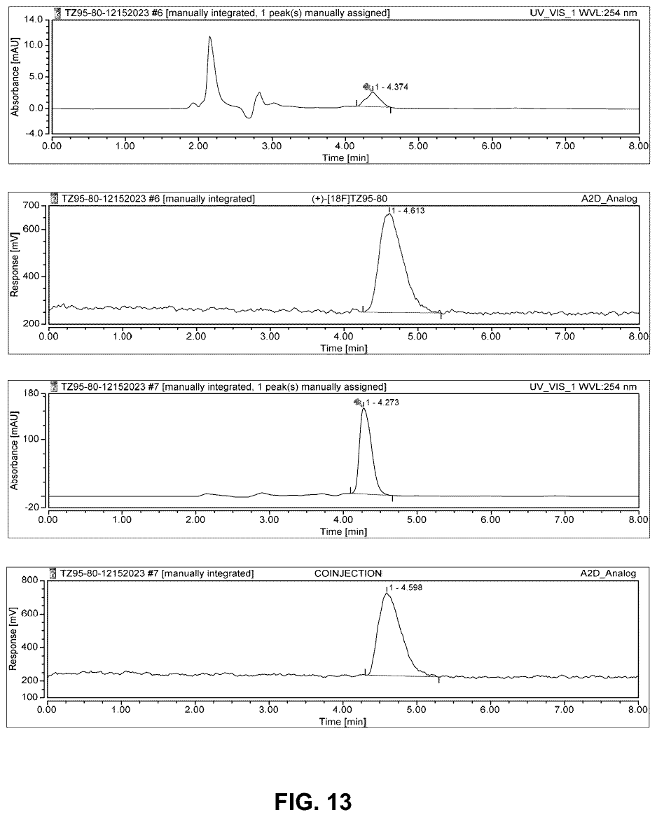

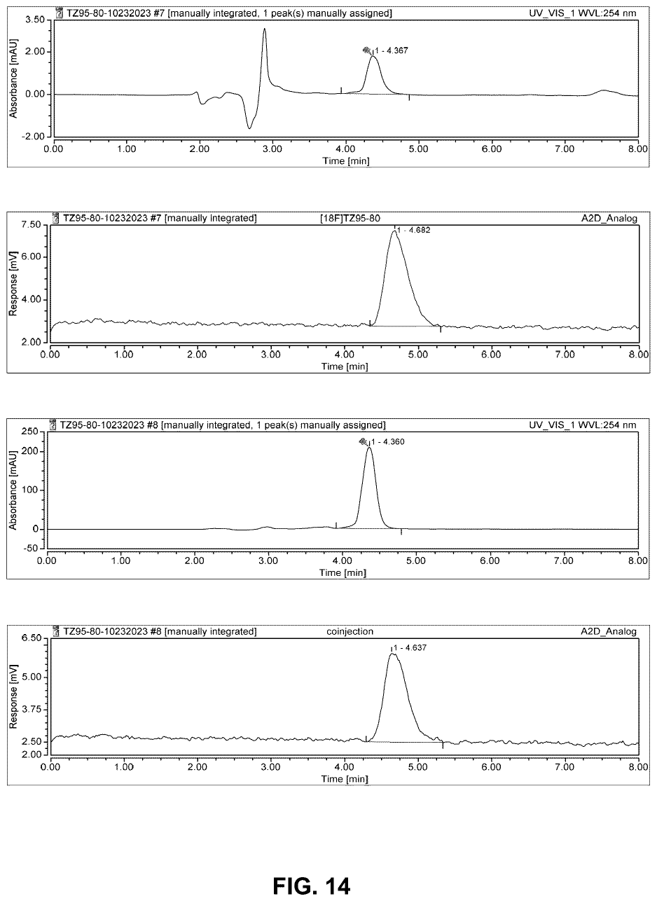

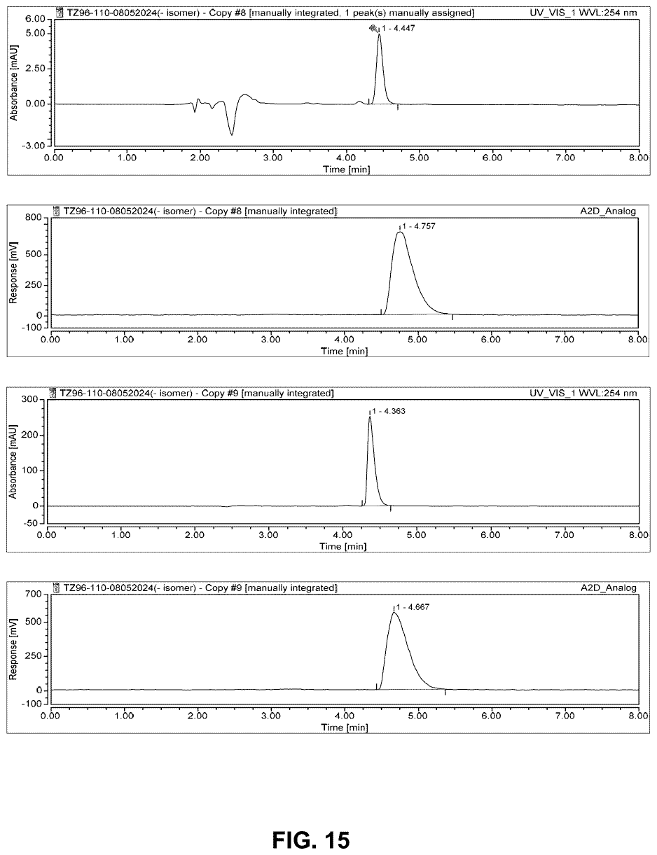

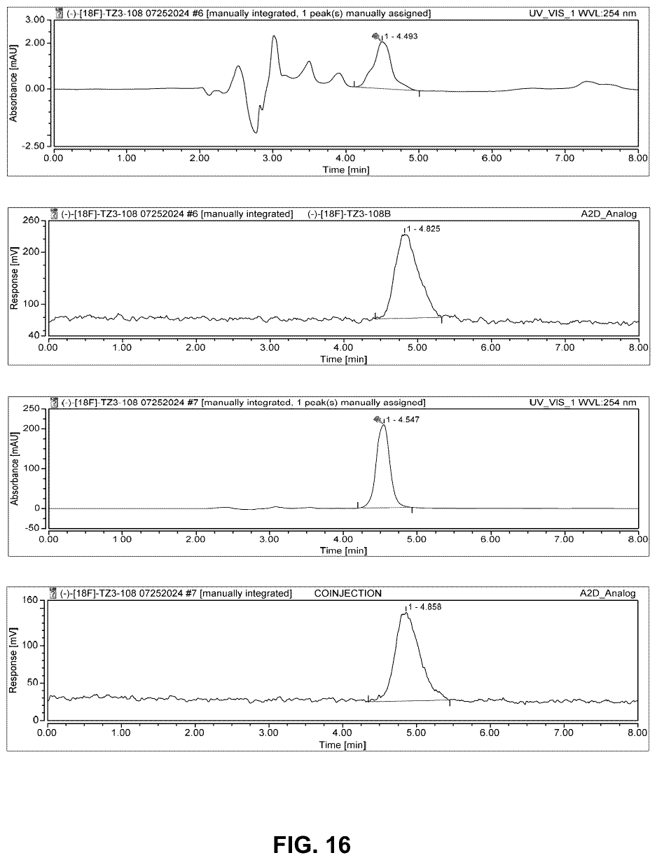

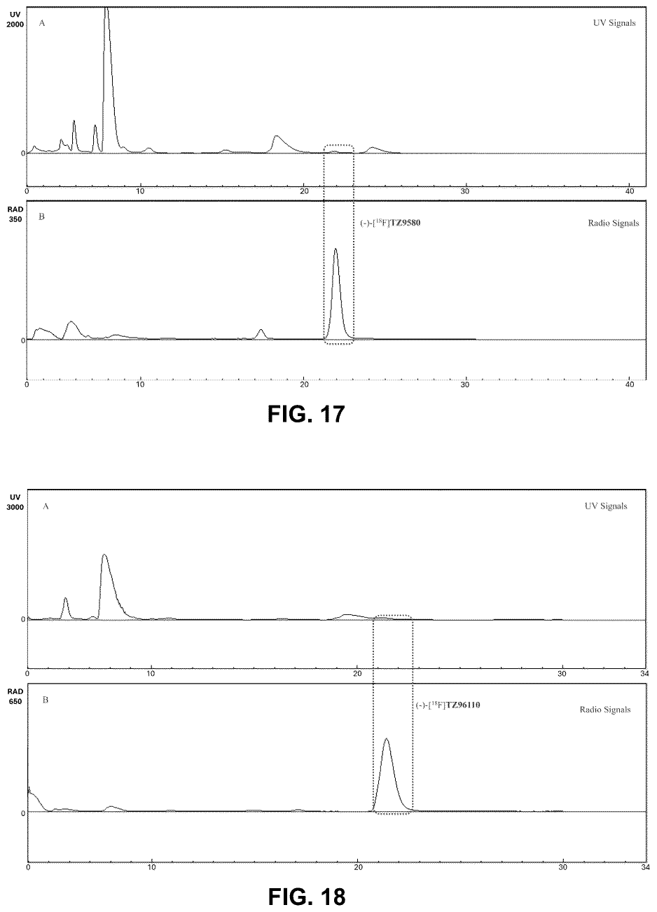

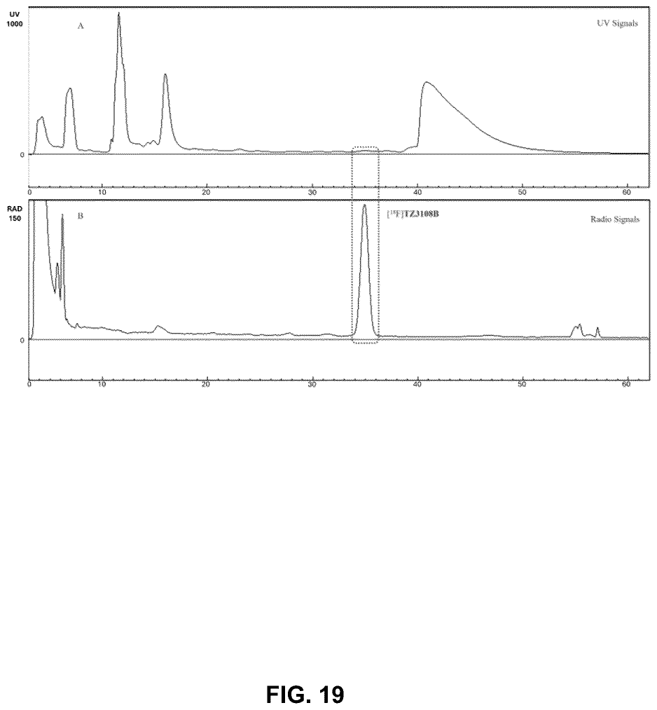

To identify the most promising F-18 labeled σ₁R radioligand for imaging σ₁R in vivo, six new F-18 radioligands were radiosynthesized, including (±)-[¹⁸F]13, (−)-[¹⁸F]13, and (+)-[¹⁸F]13, (−)-[¹⁸F]14, (−)-[¹⁸F]15, (−)-[¹⁸F]21 and the previously reported (−)-[¹⁸F]TZ3108 (FIG. 1A, FIG. 11). The analytical HPLC for quality control showed the F-18 radiochemical procedure(s) afforded all radiotracers with good yield and high radiochemical purities (FIG. 12, FIG. 13, FIG. 14, FIG. 15, FIG. 16, FIG. 17, FIG. 18, FIG. 19).

### Ex Vivo Biodistribution Studies of the σ₁R Radioligands in SD Rats.

The radioligand (−)-[¹⁸F]TZ3108 could reach equilibrium and wash out from the macaque brain, and (−)-enantiomer is more promising than (+) enantiomer for these structural analogues. To further understand the biochemical properties of the σ₁R radioligands, an ex vivo biodistribution analysis was performed for (−)-[¹⁸F]13 on adult male SD rats and compared previously published biodistribution data of (−)-[¹⁸F]TZ3108 (FIG. 1B, Table 4). Similar to (−)-[¹⁸F]TZ3108, (−)-[¹⁸F]13 entered the brain well with a % ID/g value of 0.98±0.11 at 5 min, compared to (−)-[¹⁸F]TZ3108 with uptake of 1.29±0.06, demonstrating a good BBB permeability of these structural analogues. Interestingly, compared to (−)-[¹⁸F]TZ3108, (−)-[¹⁸F]13 showed a much faster brain washout kinetics with brain uptake of 0.34±0.1, 0.30±0.03 and 0.26±0.01 at 30, 60, and 120 min. Notably, (−)-[¹⁸F]TZ3108 was accumulated in liver, spleen, pancreas, and kidney with a slow washout in these organs, whereas, (−)-[¹⁸F]13 had high uptake initially and then quickly washed out from these organs, suggesting a faster metabolic kinetics of (−)-[¹⁸F]13 compared to (−)-[¹⁸F]TZ3108. Both radiotracers showed high uptake in organs with high σ₁R expression and no defluorination was observed.

TABLE 4
Biodistribution of (—)-[¹⁸F]13 and (—)-[
¹⁸F]TZ3108 in male Sprague Dawley rats.

| | (—)-[¹⁸F]13 | | | | (—)-[¹⁸F]TZ3108⁴* | | | |
|---|---|---|---|---|---|---|---|---|
| | | | | 120 | | | | 120 |
| Organs | 5 min | 30 min | 60 min | min | 5 min | 30 min | 60 min | min |
| Blood | 0.15 ± | 0.21 ± | 0.35 ± | 0.35 ± | 0.05 ± | 0.03 ± | 0.02 ± | 0.01 ± |
| | 0.02 | 0.07 | 0.02 | 0.04 | 0.02 | 0.00 | 0.01 | 0.00 |
| Brain | 0.98 ± | 0.34 ± | 0.30 ± | 0.26 ± | 1.29 ± | 1.06 ± | 0.86 ± | 0.80 ± |
| | 0.11 | 0.1 | 0.03 | 0.01 | 0.06 | 0.13 | 0.09 | 0.13 |
| Fat | 0.10 ± | 0.15 ± | 0.23 ± | 0.22 ± | 0.02 ± | 0.08 ± | 0.09 ± | 0.10 ± |
| | 0.03 | 0.06 | 0.03 | 0.03 | 0.00 | 0.01 | 0.01 | 0.03 |
| Heart | 0.84 ± | 0.32 ± | 0.36 ± | 0.32 ± | 1.39 ± | 0.42 ± | 0.25 ± | 0.20 ± |
| | 0.05 | 0.08 | 0.03 | 0.01 | 0.24 | 0.06 | 0.03 | 0.02 |
| Kidney | 2.80 ± | 0.94 ± | 0.89 ± | 0.68 ± | 4.75 ± | 2.76 ± | 2.12 ± | 1.64 ± |
| | 0.44 | 0.25 | 0.11 | 0.06 | 0.39 | 0.09 | 0.03 | 0.21 |
| Liver | 1.89 ± | 1.32 ± | 1.32 ± | 0.88 ± | 1.62 ± | 2.76 ± | 2.84 ± | 2.81 ± |
| | 0.32 | 0.46 | 0.23 | 0.13 | 0.42 | 0.40 | 1.49 | 0.87 |
| Lung | 7.69 ± | 2.16 ± | 1.61 ± | 0.88 ± | 12.29 ± | 3.38 ± | 1.88 ± | 1.34 ± |
| | 0.47 | 0.54 | 0.2 | 0.11 | 0.86 | 0.33 | 0.38 | 0.18 |
| Muscle | 0.10 ± | 0.15 ± | 0.19 ± | 0.17 ± | 0.09 ± | 0.11 ± | 0.08 ± | 0.08 ± |
| | 0.02 | 0.05 | 0.01 | 0.01 | 0.03 | 0.03 | 0.02 | 0.00 |
| Pancreas | 1.39 ± | 0.63 ± | 0.51 ± | 0.35 ± | 1.74 ± | 1.81 ± | 1.67 ± | 1.60 ± |
| | 0.25 | 0.19 | 0.03 | 0.01 | 0.69 | 1.09 | 0.70 | 0.50 |
| Spleen | 1.87 ± | 1.60 ± | 1.28 ± | 0.85 ± | 2.52 ± | 2.83 ± | 2.71 ± | 1.88 ± |
| | 0.43 | 0.46 | 0.18 | 0.13 | 0.51 | 0.64 | 0.76 | 0.50 |
| Bone | 0.44 ± | 0.30 ± | 0.39 ± | 0.44 ± | 0.32 ± | 0.35 ± | 0.37 ± | 0.32 ± |
| | 0.06 | 0.08 | 0.04 | 0.06 | 0.01 | 0.01 | 0.04 | 0.03 |

### Autoradiograph Study of (−)-[¹⁸F]13 in CNS of Normal SD Rat Brain

To determine the brain distribution and specificity of the candidate radioligands in the brain, autoradiography of (−)-[¹⁸F]13 in brain tissues from adult male SD rats was performed (FIG. 1C, FIG. 1D). The uptake of (−)-[¹⁸F]13 was high in rat brain and the distribution matched well with immunohistochemistry staining using anti-σ₁R antibody, confirming the specificity of (−)-[¹⁸F]13. (−)-[¹⁸F]13 showed high uptakes in the gray matter-rich regions and low in the white matter-rich regions such as corpus callosum. Blocking study with both cold (−)-TZ3108 and Haloperidol, a potent σ₁R antagonist dramatically reduced the uptake of (−)-[¹⁸F]13 with nearly no uptake under blocking conditions, further confirming the specificity of (−)-[¹⁸F]13 to σ₁R.

### PET Imaging and In Vivo Specificity Confirmation of the σ₁R Radioligands in Normal Mouse Brain.

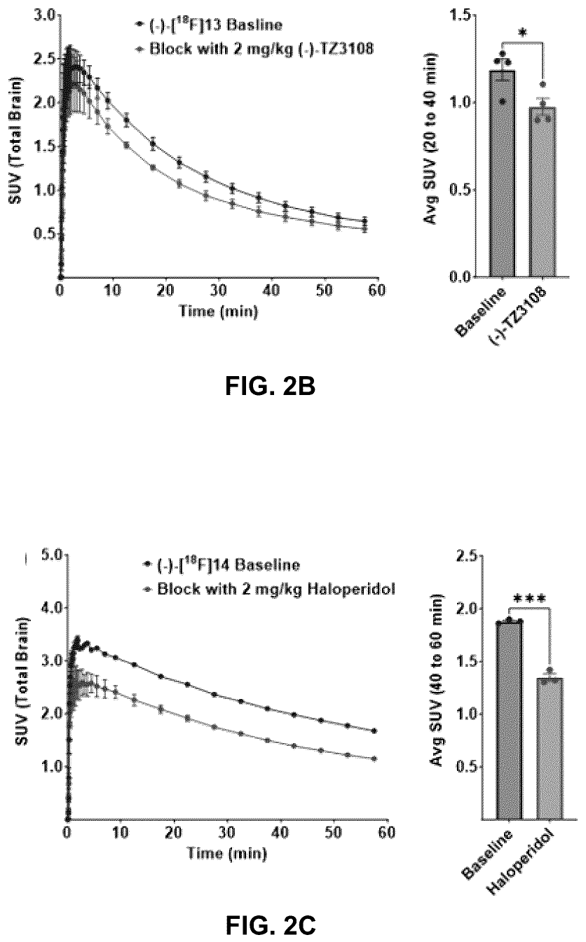

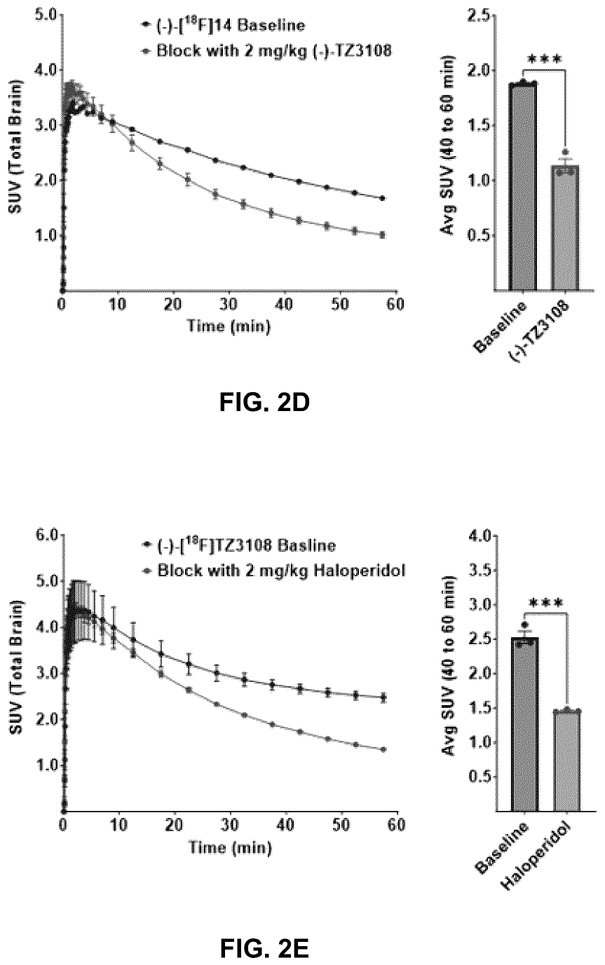

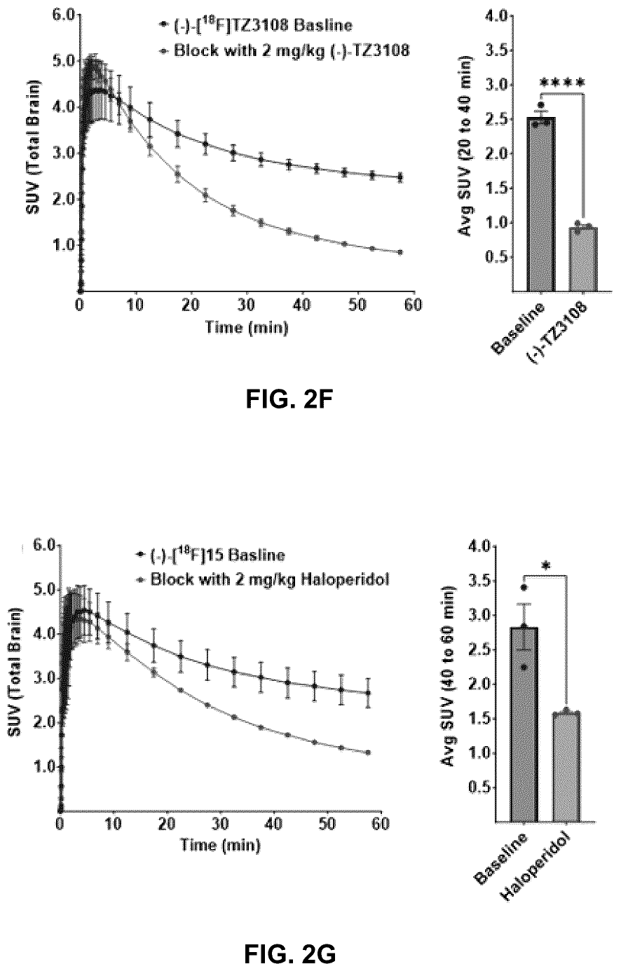

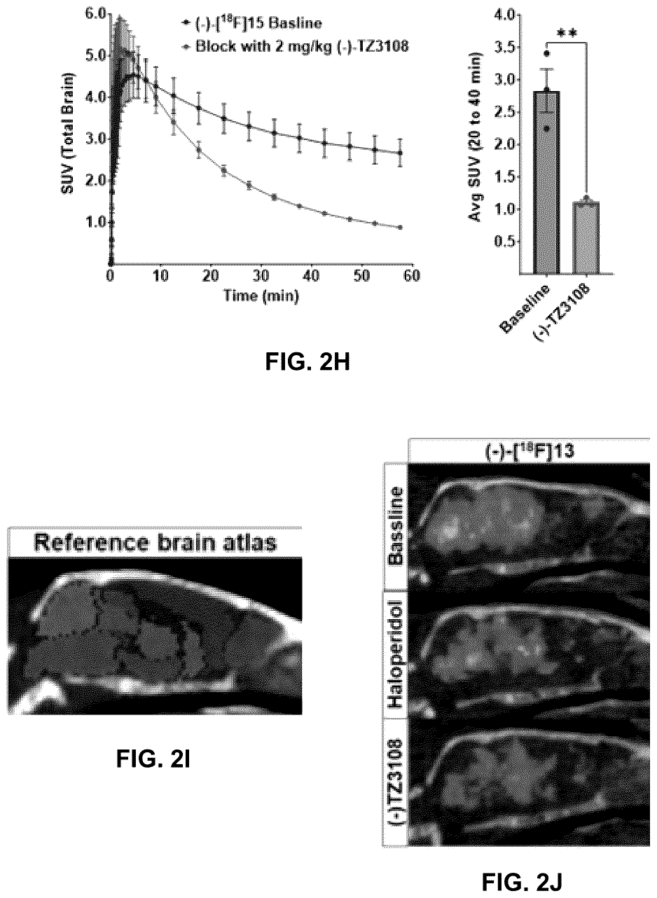

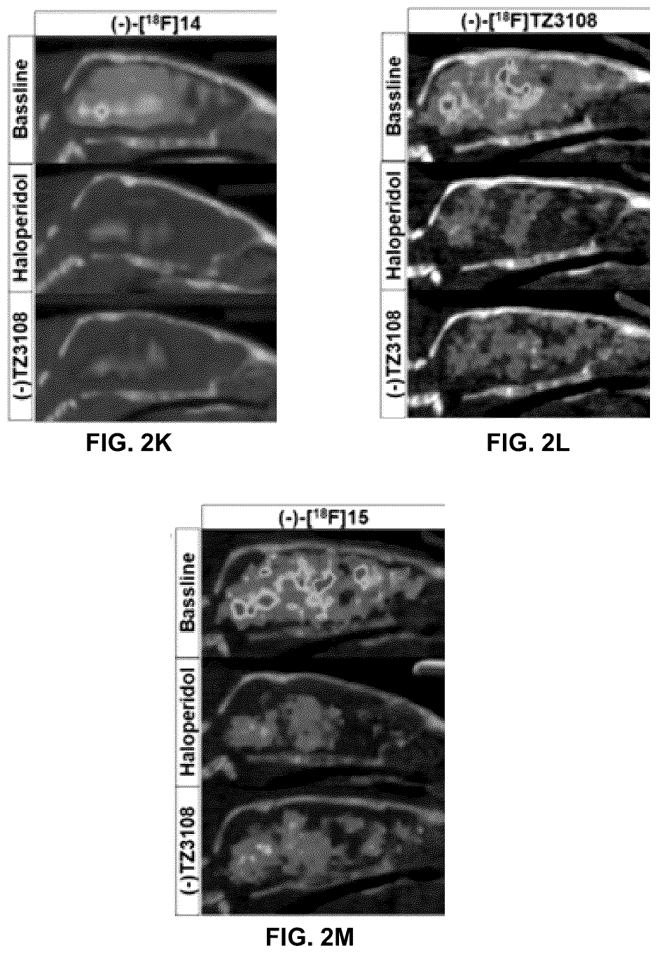

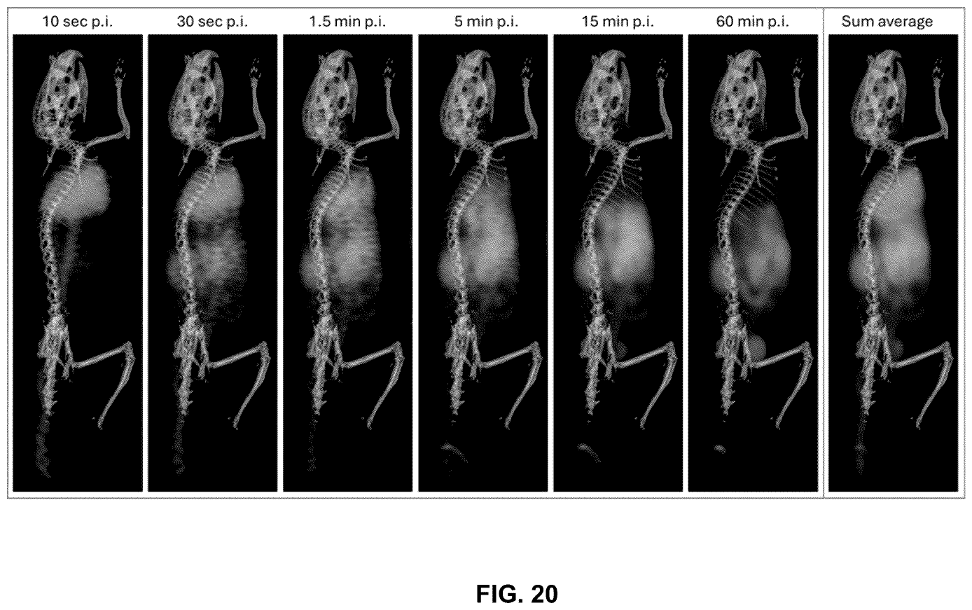

To explore the in vivo brain uptake, dynamics, and specificity of the candidate radioligands, PET imaging at baseline and blocking conditions in adult male CD-1 mice was performed (FIG. 2J, FIG. 2K, FIG. 2L, FIG. 2M). In general, all candidate radioligands including (−)-[¹⁸F]13, (−)-[¹⁸F]14, (−)-[¹⁸F]TZ3108, and (−)-[¹⁸F]15 had high brain uptakes in mouse brains. Moreover, all four radiotracers showed a good brain washout within 60 min post-injection and can be blocked with known σ₁R ligands. For example, (−)-[¹⁸F]13 quickly entered the brain and reached a peak SUV of ˜2.4 at 3 min post-injection and quickly washed out from the brain with SUVs of 2.02, 1.53, 0.91, and 0.65 at 10, 20, 40, and 60 min post-injection. Pretreatment with well-known σ₁R antagonist Haloperidol and cold (−)-TZ3108 significantly reduced the uptake of (−)-[¹⁸F]13, the average SUV from 20 to 40 min was ˜26.1% less for Haloperidol pretreated mice compared to baseline with a p-value of 0.0062 (FIG. 2A, FIG. 2B, FIG. 2J, FIG. 20). (−)-[¹⁸F]14 showed a relatively slower brain washout pharmacokinetics and a slightly higher peak of SUV of ˜3.39 at 2 min (FIG. 2C, FIG. 2D, FIG. 2K). (−)-[¹⁸F]TZ3108 (FIG. 2E, FIG. 2F, FIG. 2L) and (−)-[¹⁸F]15 (FIG. 2G, FIG. 2H, FIG. 2M) showed a slower brain washout pharmacokinetics but a higher brain uptake. (−)-[¹⁸F]TZ3108 and (−)-[¹⁸F]15 had nearly identical mouse brain washout pharmacokinetics, indicating differential labeling sites have no impact on brain pharmacokinetics. Overall, PET studies in mouse brains showed all four σ₁R radioligands have very good brain uptake and in vivo specificity. Particularly, all four radioligands showed a clear brain washout within 60 min post-injection.

PET Brain Imaging Study to Confirm the Reduction of σ₁R Expression in 3xTg-AD Mouse Brain

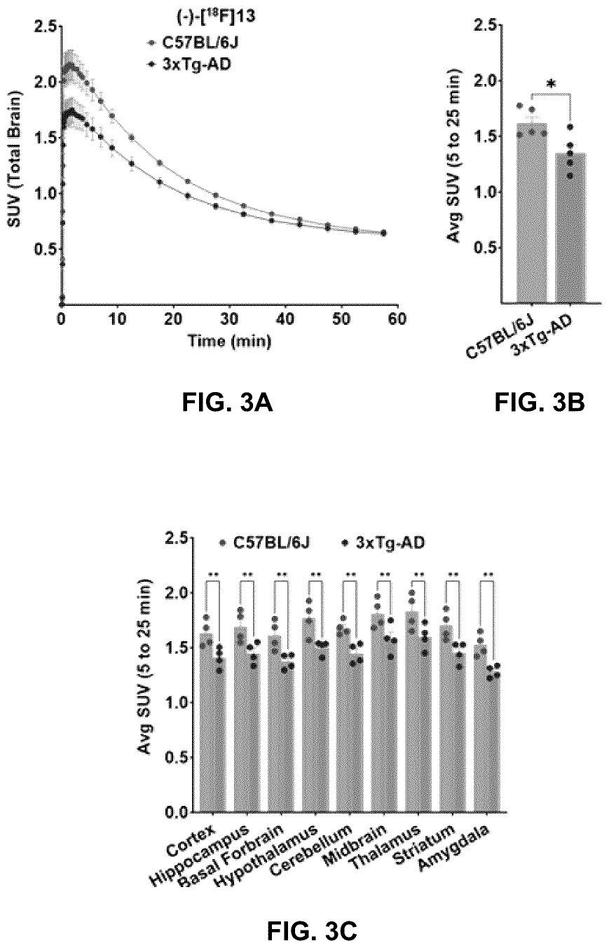

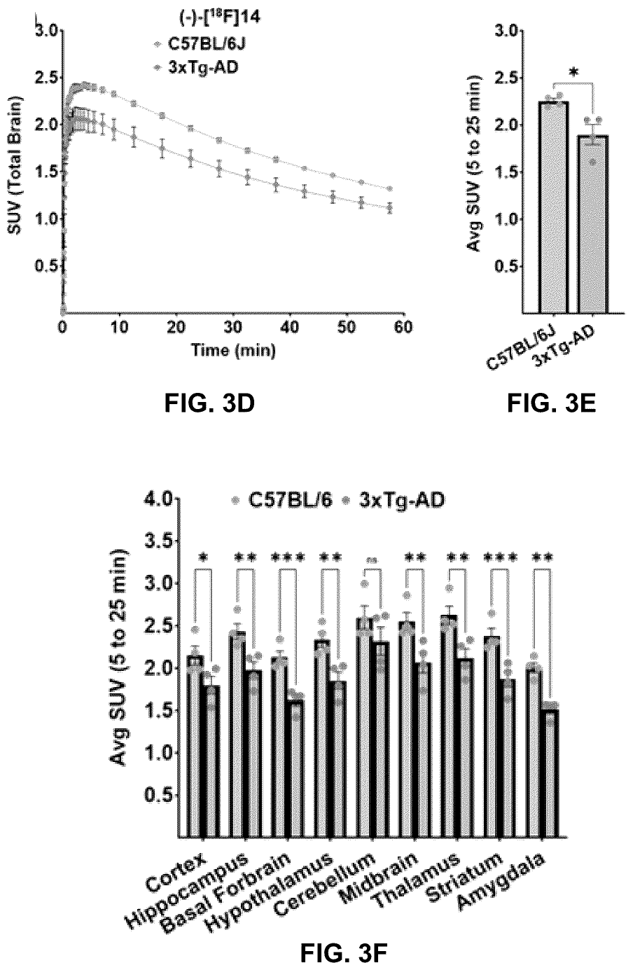

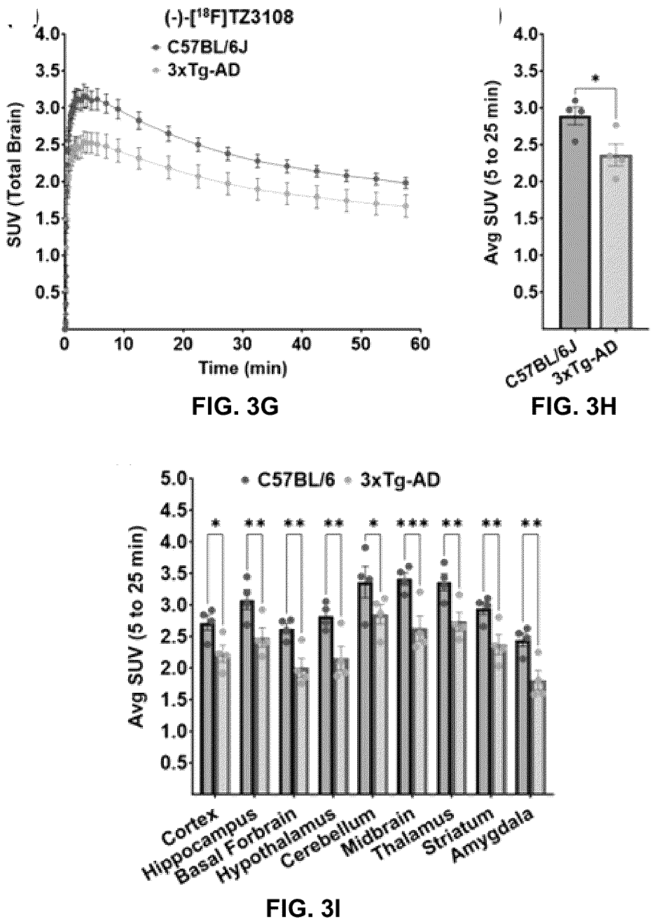

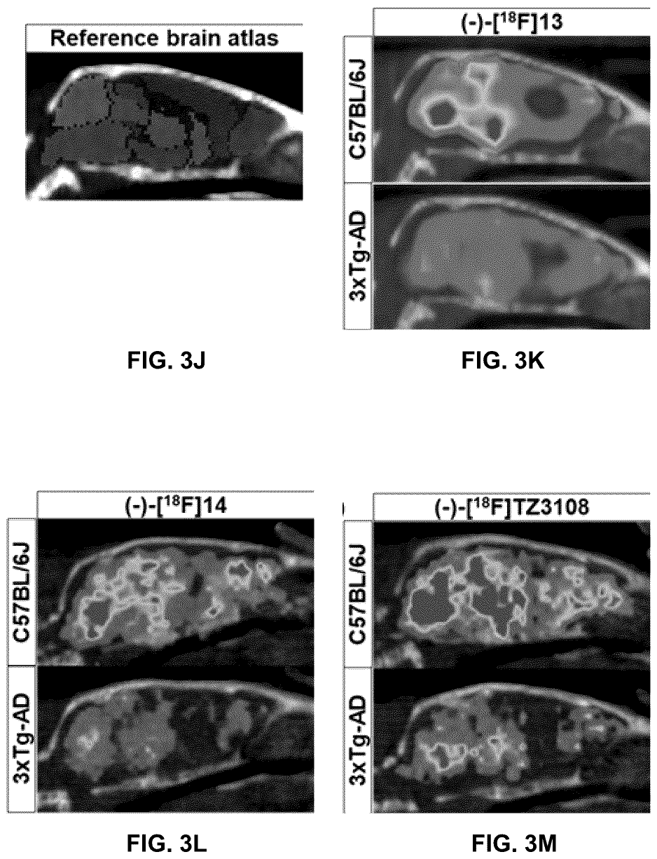

To evaluate the capability of quantifying changes in underlying AD, PET imaging studies were conducted in 11-month-old female 3xTg-AD and age-matched C57BL/6 mice. The uptake of (−)-[¹⁸F]13 was significantly reduced in the 3xTg-AD mice (Two-way ANOVA: F(1, 320)=155, P<0.0001) with a peak SUV of ˜1.75 compared to a peak SUV of ˜2.16 in controls (FIG. 3A). The average SUV from 5 to 25 min of 3xTg-AD mice was ˜1.37 compared to an average SUV of ˜1.65 in controls, a 17.0% loss in 3xTg-AD mice (P=0.0215) (FIG. 3B). Further brain regional analyses showed the uptake of (−)-[¹⁸F]13 was significantly reduced in all analyzed brain regions (Fisher's LSD test, P<0.01 for all regions) (FIG. 3C, FIG. 3K). These results indicated (−)-[¹⁸F]13 is a promising σ₁R radioligand that can quantify changes of σ₁R expression in pathological conditions. Similarly, (−)-[¹⁸F]14 showed a significantly reduced brain uptake in the 3xTg-AD mouse (Two-way ANOVA: F(1, 240)=164.3, P<0.0001) (FIG. 3D, FIG. 3E, FIG. 3F, FIG. 3L), the average brain SUV from 5 to 25 min was reduced to ˜1.90 in 3xTg-AD mice from ˜2.25 in controls, a 15.6% reduction (P=0.0179). The uptake of (−)-[¹⁸F]TZ3108 was also significantly reduced in the 3xTg-AD mice (Two-way ANOVA: F(1, 240)=268.1, P<0.0001) with a peak SUV of ˜3.15 compared to ˜2.54 in controls at ˜3 min (FIG. 3G, FIG. 3H, FIG. 3I, FIG. 3M). The average SUV from 5 to 25 min of 3xTg-AD mice reduced to ˜2.36 from ˜2.89 C57BL/6 in controls, an 18.3% reduction (P=0.0341). Overall, PET studies of the lead σ₁R radioligands showed significantly reduced brain uptakes in 3xTg-AD mouse brains compared to age-matched control mice. These results suggest the radioligands are capable of quantifying changes in σ₁R expression in pathological conditions, particularly underlying AD.

In Vitro Immunostaining Characterization of σ₁R Expression in Normal and 3xTg-AD Mouse Brain

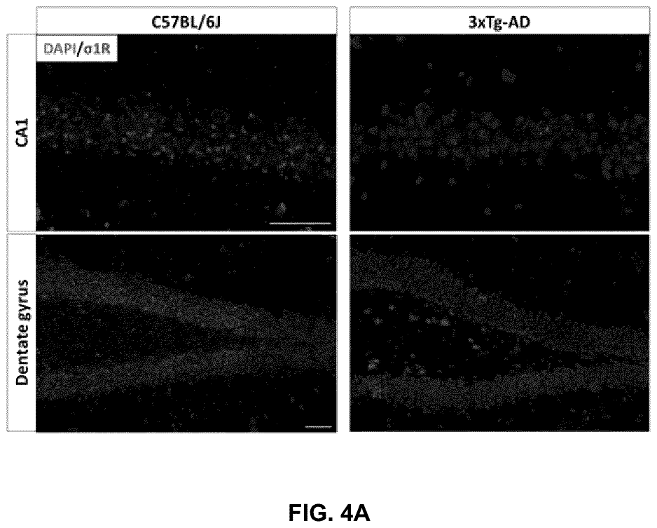

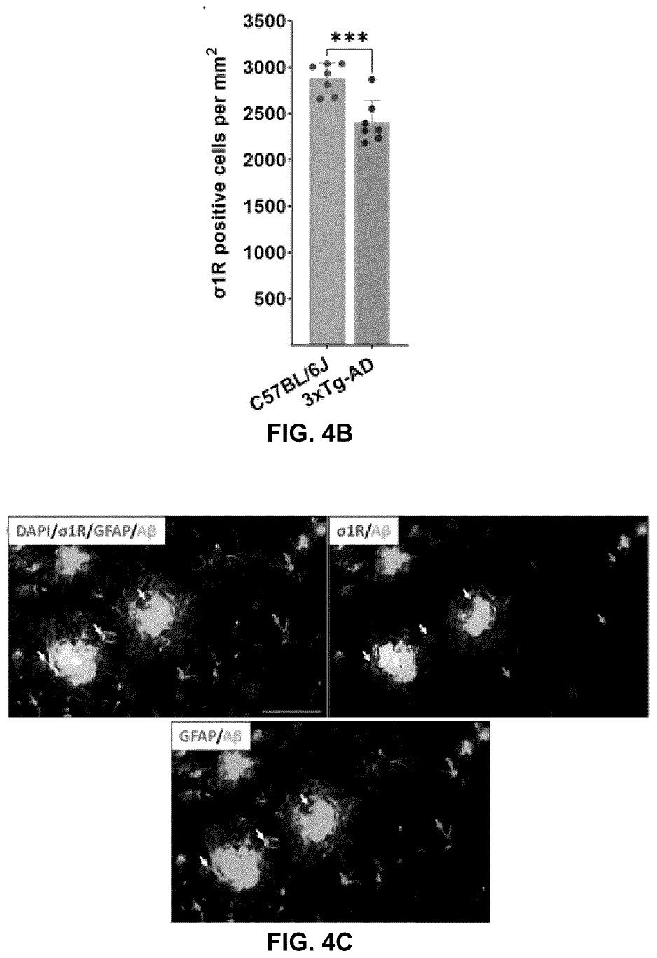

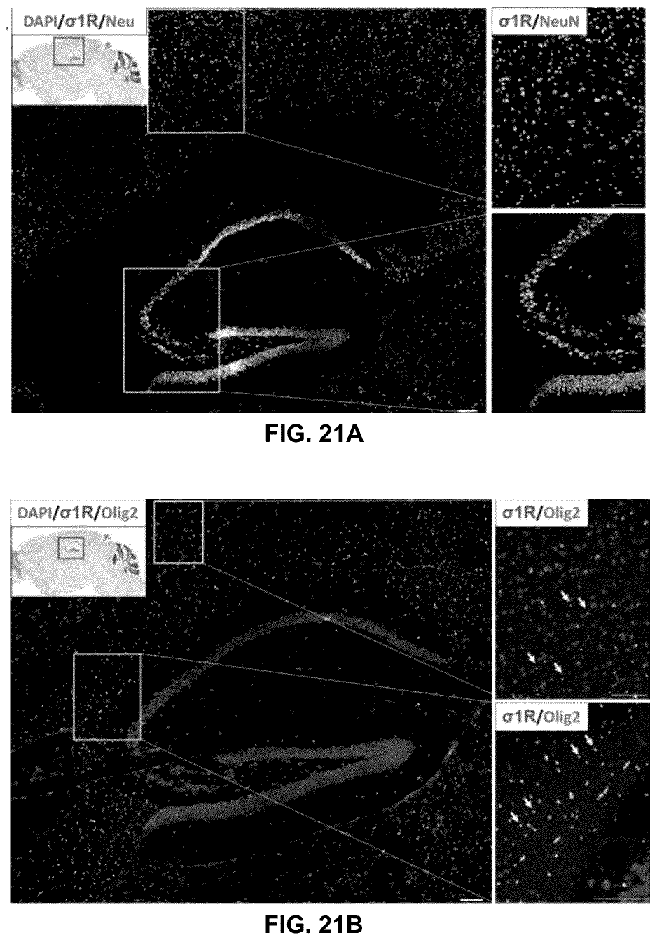

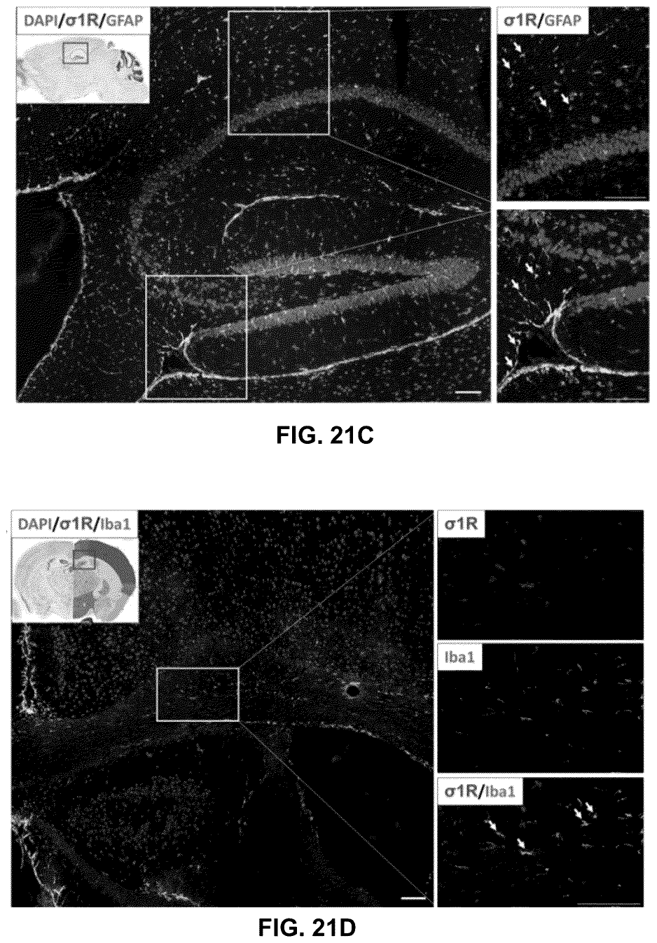

Previous studies showed σ₁R is highly expressed in the brain. However, the expression profile of σ₁R in especially in pathological condition remains unclear with different expression patterns reported. To confirm the PET findings, an immunofluorescent analysis was performed in mouse brain tissues. The study showed that the expression of σ₁R was high in brain cells, including neurons, oligodendrocytes, astroglia, and microglia (FIG. 21A, FIG. 21B, FIG. 21C, FIG. 21D). Moreover, consistent with the PET findings, a significant loss of σ₁R-positive neurons was identified in the hippocampus of 3xTg-AD mouse brains (FIG. 4A). Quantitative analysis showed the number of σ₁R-positive neurons in the CA1 region of the hippocampus was 2878/mm² in C57BL6J mice in compared to a total of 2407/mm² σ₁R-positive neurons in 3xTg-AD mice, a 16.4% loss with a p-value of 0.0010 (FIG. 4B). Interestingly, σ₁R was also expressed in a portion of astrocytes (FIG. 21C). While the overall expression of σ₁R was low in 3xTg-AD mice, σ₁R was highly expressed in a group of GFAP-positive astrocytes that surround Aβ plaques, and the σ₁R expression was low in astrocytes that were distanced from Aβ plaques (FIG. 4C). This indicates σ₁R may be involved in the activation of astrocytes in pathological conditions, particularly in the Aβ pathology-associated AD mechanisms.

### PET Brain Imaging Study in Cynomolgus Macaque

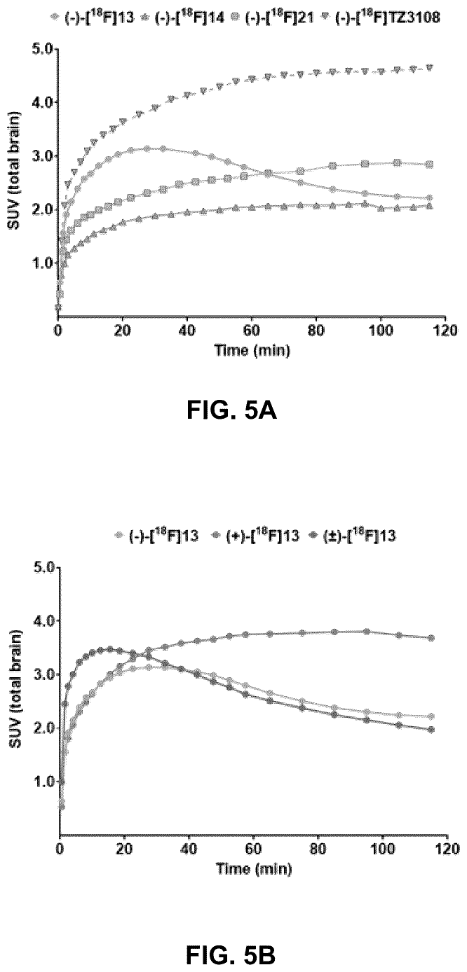

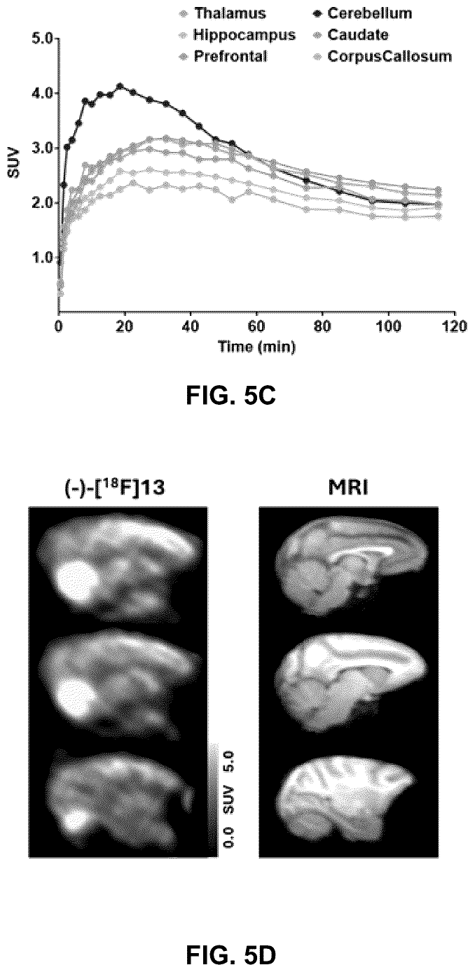

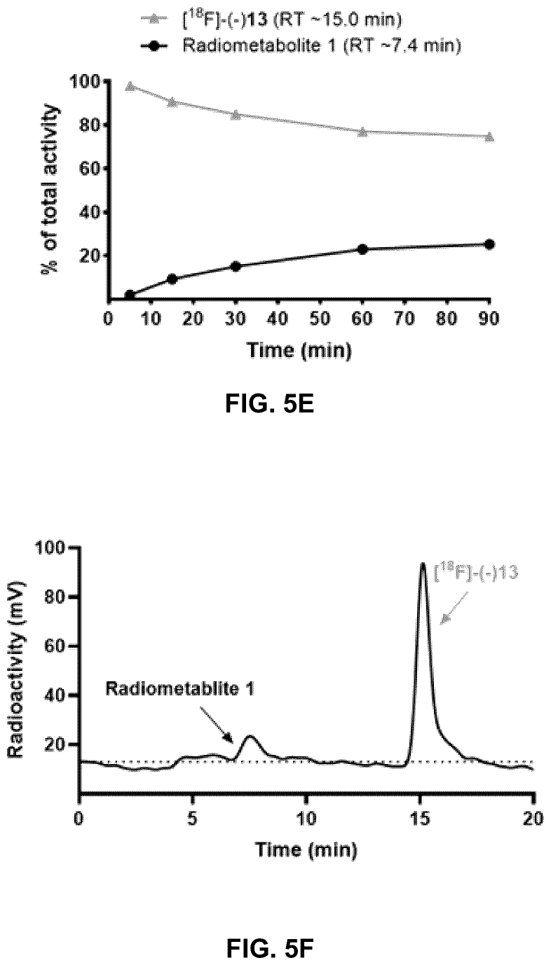

Previously published σ₁R radioligands from other groups show limitations in clinical stages, with difficulties reaching equilibrium within hours post-injection. While the radioligands showed clear washout in rodents, the major goal is to identify clinically suitable radioligand(s) to precisely quantify σ₁R expression in human brains. Therefore, PET studies were performed in non-human primates, which provide better anatomical and physiological similarities to the human brain compared to rodents. The uptake and radiopharmacokinetics of (−)-[¹⁸F]13, (−)-[¹⁸F]14, (−)-[¹⁸F]TZ3108, and (−)-[¹⁸F]21 was compared (FIG. 5A). (−)-[¹⁸F]13 entered the brain quickly and achieved a peak SUV of ˜3.14 at 27.5 min post-injection, and gradually washed out from the brain with SUVs of 3.06, 2.80, 2.30, and 2.22 at 40, 60, 100, and 120 min. (−)-[¹⁸F]TZ3108 also entered the brain fast and showed the highest SUV among the tested radioligands with a peak SUV of ˜4.64 at 120 min post-injection. (−)-[¹⁸F]14 showed similar tracer kinetics as (−)-[¹⁸F]TZ3108 but a relatively lower brain uptake with a peak SUV of ˜2.10 at 95 min post-injection. (−)-[¹⁸F]21 entered the brain fast and showed a relatively slow brain washout kinetics with a peak SUV of ˜2.87 at 105 min post-injection. Among all tested radioligands, (−)-[¹⁸F]TZ3108 had the highest brain uptake followed by (−)-[¹⁸F]13, (−)-[¹⁸F]14, and (−)-[¹⁸F]21. However, in contrast to rodent studies where all radioligands reached equilibrium and washout from the brain fast, only (−)-[¹⁸F]13 showed a clear brain washout kinetics in macaque, though other radioligands also seemed to reach the equilibrium and retain in the brain during 120 min post-injection.

When comparing (−)-[¹⁸F]13 with its racemic enantiomer (+)-[¹⁸F]13 and racemic mixtures (±)-[¹⁸F]13 in macaque brains (FIG. 5B), (+)-[¹⁸F]13 had a similar high brain uptake with a peak SUV of ˜3.80 but with much slower brain washout pharmacokinetics. The racemic mixtures (±)-[¹⁸F]13 showed the combined molecular as expected. These interesting results suggest that the chiral structure of compound 13 is important to the molecular interaction with σ₁R protein and can impact the brain washout pharmacokinetics, consistent with the finding in TZ3108 that the (−)-enantiomer radiotracer is more favorable than (+)-enantiomer^{22,23}. Detailed subregional analysis showed (−)-[¹⁸F]13 has the highest uptake in cerebellum with a peak SUV of ˜4.12 at 18.5 min post-injection and was high in gray matter-rich regions such as thalamus, frontal cortex, and hippocampus, and low in the white matter-rich regions such as corpus callosum (FIG. 5C, FIG. 5D). The distribution of (−)-[¹⁸F]13 in macaque brains matched well with in vitro autoradiography and immunohistology analysis (FIG. 1B, FIG. 1C), further confirming the specificity of (−)-[¹⁸F]13. Overall, PET studies in macaque brains showed that all candidate radioligands have high brain uptake with (−)-[¹⁸F]13 showing the most clinically favorable brain washout pharmacokinetics.

### Radiometabolite Analysis of Radiotracers in Macaque and Rat.

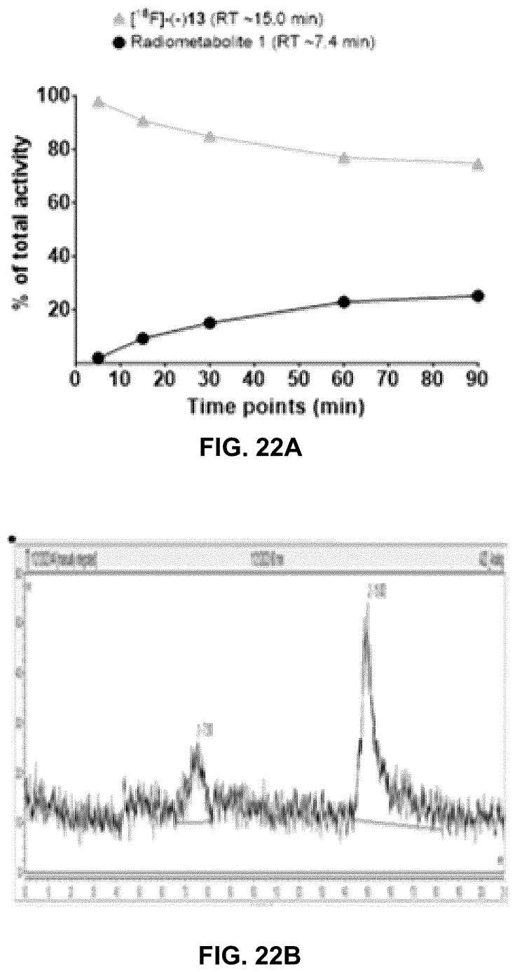

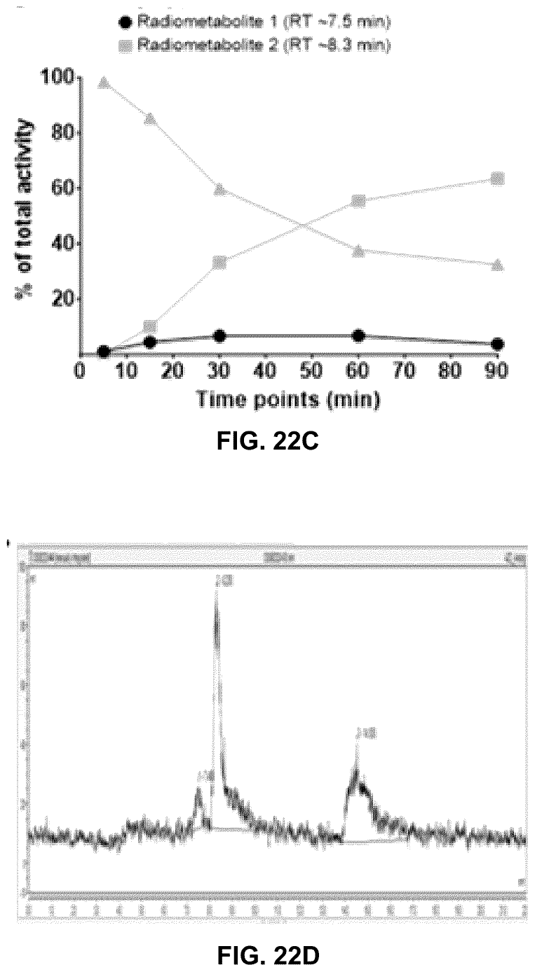

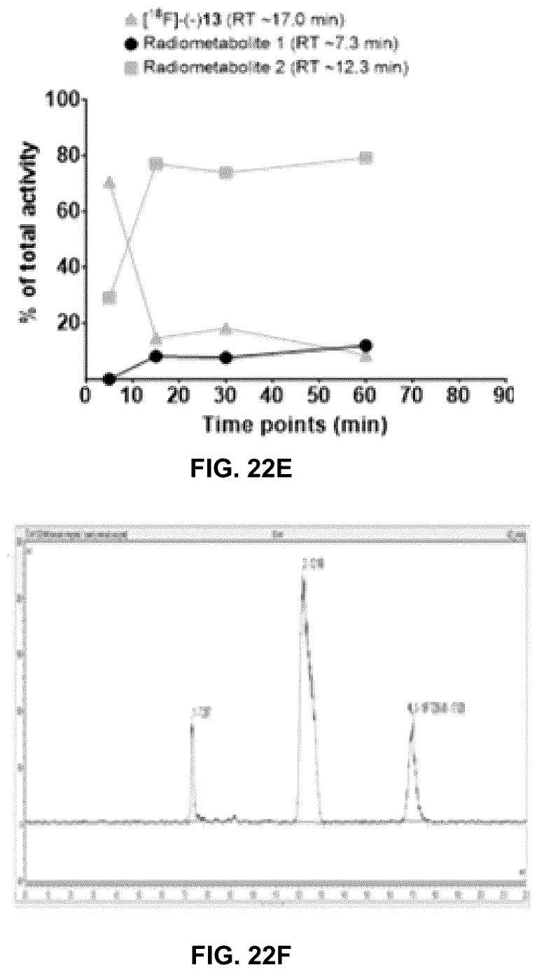

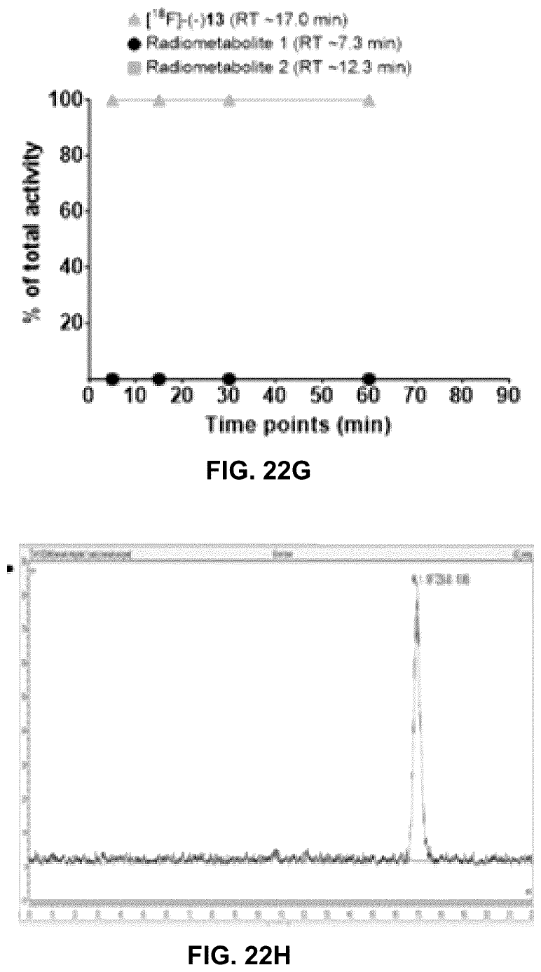

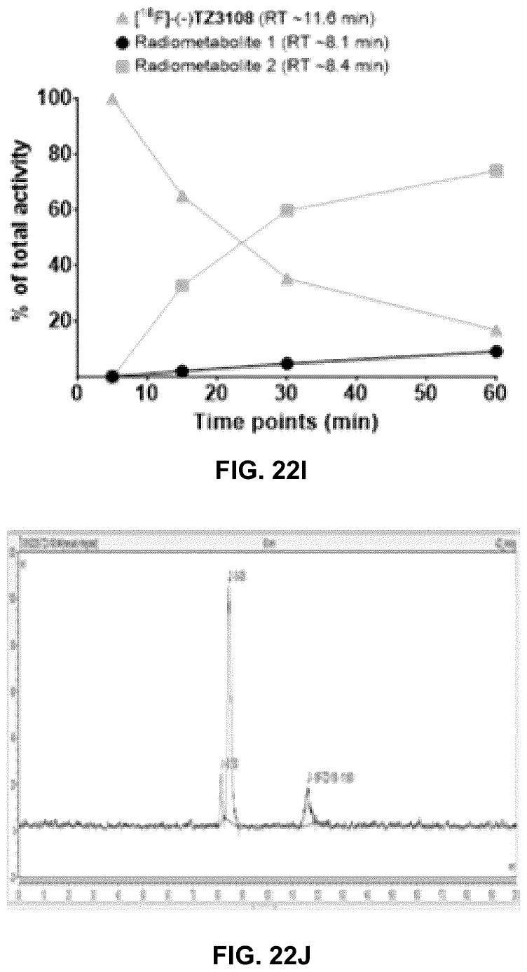

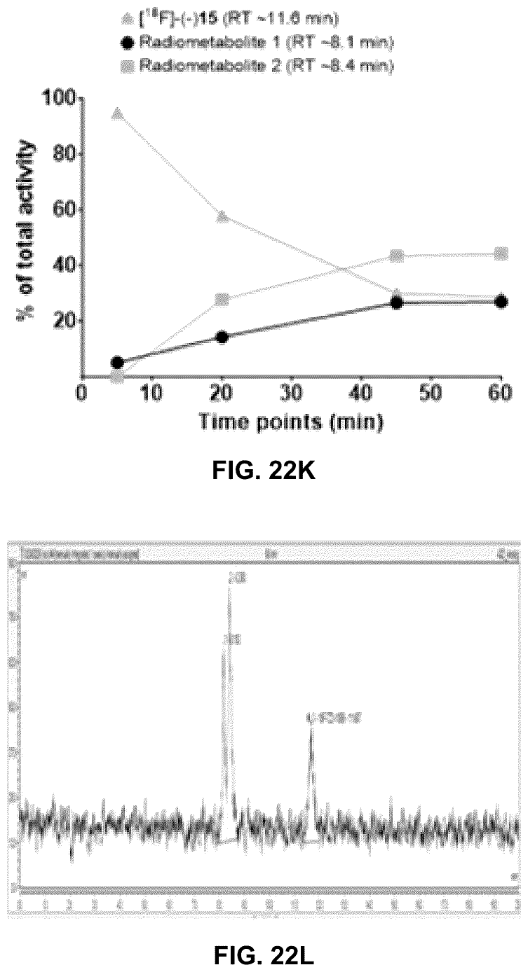

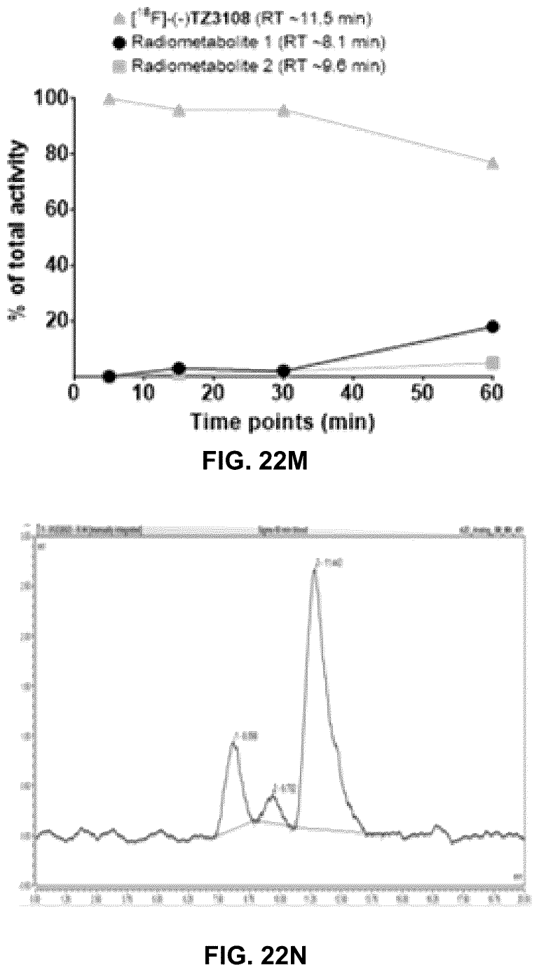

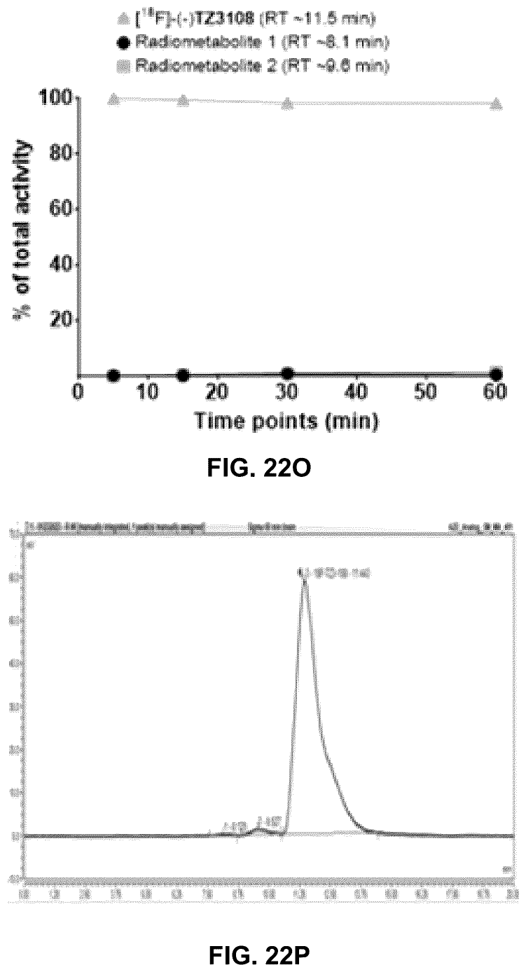

To understand the pharmacokinetics of the σ₁R radiotracers and to determine if radiometabolite can enter the brain and confound the PET quantification, radiometabolite analysis was conducted focusing on the lead radioligands (−)-[¹⁸F]13 and (−)-[¹⁸F]TZ3108 in macaque plasma, and rat plasma and brain. For (−)-[¹⁸F]13, HPLC plasma radiometabolite analysis showed a slow metabolism with the amount of parental radiotracer at 98.02%, 90.74%, 84.92%, 77.07%, at 74.97% at 5, 15, 30, 60, and 90 min, respectively. Only one hydrophilic radiometabolite was identified (FIG. 5E, FIG. 5F), indicating a good in vivo stability of (−)-[¹⁸F]13 in macaque. To determine if this radiometabolite can enter the brain, HPLC radiometabolite analysis was performed on rat plasma and brain homogenates (FIG. 22E, FIG. 22F, FIG. 22G, FIG. 22H). In rat plasma, two hydrophilic radiometabolites and a much faster metabolism rate were observed, indicating a differential metabolism of (−)-[¹⁸F]13 between rat and macaque. Importantly, in the rat brain homogenate, only the parental radiotracer (−)-[¹⁸F]13 was observed, indicating neither of two radiometabolites identified in plasma can enter the brain (FIG. 22G, FIG. 22H). To understand the differential pharmacokinetics in macaque PET studies, a HPLC-radiometabolite analysis of (+)-[¹⁸F]13 in macaque plasma was also performed. Two radiometabolites and a much faster radiometabolism were identified compared to (−)-[¹⁸F]13 (FIG. 22C, FIG. 22D), indicating a chiral structure-dependent metabolism of [¹⁸F]13. Overall, the radiometabolite analysis of (−)-[¹⁸F]13 demonstrates a good in vivo stability of (−)-[¹⁸F]13 with no radiometabolite entering the brain. Similarly, (−)-[¹⁸F]TZ3108 also showed relatively good stability in vivo in macaque plasma (FIG. 22I, FIG. 22J). Moreover, (−)-[¹⁸F]TZ3108 was more stable in rat than in macaque plasma (FIG. 22M, FIG. 22N) and no radiometabolite was identified in rat brain (FIG. 22O, FIG. 22P), In summary, (−)-[¹⁸F]13 was more stable than (−)-[¹⁸F]TZ3108, with both of the lead radiotracers showing no radiometabolites entering the brain.

### Discussion

Evidence demonstrated an abnormal function of σ₁R in AD, and drugs targeting σ₁R can reduce the AD pathology and cognitive decline in animal models of AD, suggesting σ₁R is a promising biomarker for early AD diagnosis and progression, as well as monitoring disease-modifying therapeutics. Several PET radioligands have been developed with different limitations such as low selectivity and specificity, irreversible binding profiles, difficulty in reaching equilibrium within hours, and low in vivo specificity, and thus controversial results have been reported with the same tracer in different studies. To continue the development of clinically suitable σ₁R radioligands and to explore the abnormal expression of σ₁R in AD, the present study has further characterized the σ₁R radioligand (−)-[¹⁸F]TZ3108. To extend understanding of molecular properties of (−)-[¹⁸F]TZ3108, were also introduced as an alternative radiolabeling approach and obtained a new radioligand (−)-[¹⁸F]15. Additionally, this study has successfully developed three σ₁R radioligands (−)-[¹⁸F]13, (−)-[¹⁸F]14, and (−)-[¹⁸F]21 (FIG. 1A). In optimized synthesis and radiosynthesis approaches of these new ligands achieved a good yield and high purities. Compared to TZ3108, these ligands retained comparable potency and selectivity for σ₁R with slight compromises (Table 3). Notably, previous studies suggested σ₁R ligands with slightly lower affinity may have more favorable brain washout pharmacokinetics.

The PET brain imaging studies in mice at baseline and blocking conditions demonstrated that all the candidate radioligands have good brain uptake, brain washout pharmacokinetics, and in vivo specificity (FIG. 2J, FIG. 2K, FIG. 2L, FIG. 2M). As expected, (−)-[¹⁸F]15 showed a nearly identical brain uptake and kinetics as (−)-[¹⁸F]TZ3108, indicating alternative ¹⁸F-radiolabeling position has no impact on the molecular properties. In contrast, introducing [¹⁸F]fluoroethoxy group significantly impacts the brain uptake, (−)-[¹⁸F]14 has a lower brain uptake with similar kinetics compared to (−)-[¹⁸F]TZ3108. (−)-[¹⁸F]13 with a 1,3′-bipiperidin group instead of 1,4′-bipiperidin group showed an even lower brain uptake but also faster kinetics. Biodistribution studies in rats showed (−)-[¹⁸F]13 has lower brain uptake and faster washout from all major organs, whereas (−)-[¹⁸F]TZ3108 has a relatively slower washout in these organs (FIG. 1B, Table 4). Radiometabolite study in macaque plasma (FIG. 5E, FIG. 5F, FIG. 22A, FIG. 22B, FIG. 22C, FIG. 22D) showed (−)-[¹⁸F]13 had a much slower metabolite rate compared to (−)-[¹⁸F]TZ3108. Previous studies reported a decrease of σ₁R expression in AD patients and mouse models of AD. The PET studies with (−)-[¹⁸F]TZ3108, (−)-[¹⁸F]14, and (−)-[¹⁸F]13 showed a reduced brain uptake in the 3xTg-AD mice compared to controls (FIG. 3K, FIG. 3L, FIG. 3M). While (−)-[¹⁸F]TZ3108 has the highest brain uptake, (−)-[¹⁸F]13 has a faster brain washout kinetics than (−)-[¹⁸F]TZ3108. The slightly lower brain uptake of (−)-[¹⁸F]13 does not impact its capability to distinguish the difference of either total brain uptake or the brain regional uptake, and all tested radioligands showed promise in quantification of the decreased σ₁R expression in 3xTg-AD mice. Surprisingly, in contrast to PET studies in rodents, PET studies on macaque brains showed only (−)-[¹⁸F]13 washout from the macaque brain significantly, other radioligands including (−)-[¹⁸F]TZ3108, (−)-[¹⁸F]14, and (−)-[¹⁸F]21 seemed to reach the equilibrium but were retained in the brain during 120 min post-injection. Since non-human primate brain provides better anatomical and physiological similarities to the human brain compared to rodents, the data suggests (−)-[¹⁸F]13 is more promising for further clinical investigation.

To confirm the PET findings, in vitro analysis was conducted in rodent tissues. The autoradiography study with the lead radioligand (−)-[¹⁸F]13 confirmed its high brain uptake and its specificity. Consistent with previous findings, it was found that σ₁R is highly expressed in the mouse brain, particularly highly distributed in neurons. σ₁R is also expressed in all oligodendrocyte cells at relatively lower level. Interestingly, σ₁R is present in a portion of astrocytes and microglia cells, indicating σ₁R may be involved in the activation/deactivation of these neuroinflammatory-mediated cells and is only present in certain stages of these cells (FIG. 21A, FIG. 21B, FIG. 21C, FIG. 21D). The study in 3xTg-AD mouse revealed a reduced σ₁R expression and number of σ₁R positive neurons in the brain of 3xTg-AD mouse (FIG. 4B), consistent with previous studies using western blot and other methods and unveils the main contribution to lower expression in AD is from the neuronal σ₁R loss. This also may explain the positive impact of σ₁R agonist in 3xTg-AD mice. It is hypothesized that neuroprotective effects of σ₁R play a crucial role in the pathogenesis and progression of neurological disorders. The loss of σ₁R in neurons may reduce its neuroprotective function and contribute to the disease progression. Furthermore, a group of σ₁R-positive Aβ-associated astrocytes was identified, σ₁R seems elevated only in astrocytes that surround to Aβ plaque but not in those distant from Aβ plaques. This suggests σ₁R may be involved in the regulation of astrocyte activities and tightly related to Aβ pathology.

In summary, σ₁R plays a pivotal role in the pathogenesis of AD, PET with a suitable radiotracer could provide a powerful tool for quantifying σ₁R in early AD and also for validating the therapeutic efficacy. A group of F-18 labeled σ₁R-specific radioligands were developed and characterized, all of which are potent, selective, and specific to σ₁R and can successfully quantify changes of σ₁R expression in AD mouse model. The data suggested that (−)-[¹⁸F]13 is the most promising σ₁R radiotracer with high brain uptake, good in vivo specificity and stability, and clinically favorable brain washout pharmacokinetics that overcome issues with previous reported σ₁R radioligands. Future toxicity validation is warranted to seek FDA approval for human use.

### Methods

### Animals

Wild-type Sprague Dawley (SD) rat and CD-1 IGS mouse (Charles River Laboratory, Wilmington, MA), 3xTg-AD and age-matched C57BL/6J mice (The Jackson Laboratory, Bar Harbor, ME), and Macaca fascicularis (Tulane National Primate Research Center) were purchased and kept in the animal facilities.

### Chemicals

All reagents and chemicals were acquired commercially and used as received. Procedures for synthesis and radiosynthesis were described in detail in Supplemental Materials. Briefly, racemic σ₁R compounds, (±)-13, (±)-14, (±)-15, and (±)-21 and their enantiomers were successfully synthesized and resolved (FIG. 7, FIG. 8, FIG. 11). The design strategies to optimize pharmacokinetic properties of novel σ1R compounds included: (1) replacing the fluorobenzyl group on TZ3108 using a fluoroethoxy benzyl group on 14 and 21; (2) replacing 1,4′-bipiperidin on TZ3108 and 14 using 1,3′-bipiperidin on 13; (3) replacing [¹⁸F] from benzoyl group in TZ3108 to fluorobenzyl group in 15. Radiosynthesis of corresponding radioligands was successful with a radiochemical purity of >99%, chemical purity of >95%, and molar activity from 48 to 93 GBq/μmol.

### In Vitro Competition Binding Assay

In vitro binding affinity assay was performed to determine the potency and selectivity of candidate compounds. Briefly, σ₁R assay was carried out using adult SD rat brain membrane homogenates and (+)-[³H]pentazocine (Revvity, Waltham, MA). Dilutions of candidate compounds from ˜400 pM to 100 μM were prepared in assay buffer containing 150 mM NaCl, 100 mM EDTA, 0.1% saponin, 0.1% Triton X-100, and 0.5% BSA in 50 mM Tris-HCl and incubated with ˜500 μg of brain membrane homogenate and 5 nM (+)-[³H]pentazocine, and then filtered and washed 3 times with ice-cold buffer. The bound (+)-[³H]pentazocine was counted using a liquid scintillation counter (Beckman, Brea, CA). Nonspecific binding was determined by adding 10 μM haloperidol. Similarly, σ2R binding assay was carried out using rat liver membrane homogenates and [³H]DTG (Revvity, Waltham, MA). Vesicular acetylcholine transporter (VAChT) binding assay was performed using post-nuclear lysate from PC12^{A123.7} that expresses human VAChT protein and (−)-[³H]vesamicol (Revvity, Waltham, MA). A nonlinear regression one-site binding model was used to determine the Ki.

### Ex Vivo Biodistribution Analysis in SD Rats

Biodistribution studies were performed. Briefly, adult male SD rats (˜250 grams) were used, ˜1.5 MBq radioligand was administered to the animal intravenously under anesthesia. Rats were euthanized at 5-, 30-, 60-, and 120-minute post-injection (n=4 per group) and tissues of interest, including blood, lung, kidney, liver, spleen, heart, pancreas, muscle, fat, and brain were collected, weighed, and counted on an automated gamma counter (Beckman, Brea, CA). The tracer uptake in each sample was calculated as weight, background, and decay-corrected percent injected dose per gram (% ID/g).

### In Vitro Autoradiography Analysis

Autoradiography was carried out using frozen brain sections from adult SD rats as described before with minor modifications. Briefly, 20 μm fresh frozen tissue sections were pre-incubated with HBSS buffer containing 10 mM HEPES, 5 mM MgCl₂, 0.5% BSA, and 0.1 mM EDTA at pH 7.4 for 5 min. Sections were then incubated with ˜740 kBq of radioligand and washed with ice-cold buffer, dipped in ice-cold H₂O, and air dried with a blower. Dried slides were incubated with a BAS-IP MS Storage Phosphor Screen (Cytiva, Marlborough, MA). The autoradiography signal was measured using a Typhoon FLA 9000 scanner (Fuji, Tokyo, Japan). Images were quantified using Multi Gauge v3.0 software. The specificity of candidate radioligands was determined by the addition of 10 μM of desired σ₁R compounds.

### PET Brain Imaging in Mouse

PET studies in mice were carried out using a Mediso nanoScan imager. Anatomical data for co-registration were obtained from a CT scan, and a 60 min dynamic emission scan was acquired after administration of an average of ˜7.4 MBq of each radioligand. The reconstructed PET image was analyzed using Imalytics Preclinical software 3.0 (Gremse-IT GmbH, Aachen, Germany). ROIs including total and subregions of the brain were obtained using ‘Mouse Brain Benveniste Mirrione’ mouse brain atlas. The radioligand uptake was calculated in standard uptake value (SUV). Eight-week-old male CD-1 IGS mice were used for the evaluation brain uptakes under baseline and blocking conditions. Blocking agents were administered 5 min before the dose. Eleven-month-old female 3xTg-AD mice and age-matched C57BL/6J mice were used for the evaluation of changes in σ₁R expression underlying AD.

### In Vitro Histology and Immunohistochemistry

Immunohistochemistry of σ₁R was performed in frozen sections from adult SD rat brains. Briefly, sections were fixed in 4% paraformaldehyde and blocked with BLOXALL (Vector Laboratories, Burlingame, CA). Antigen retrieval was performed with Antigen Unmasking Solution in a steamer. Sections were then blocked with 5% horse serum, stained with anti-σ₁R antibody and ImmPRESS HRP Horse anti-rabbit polymer and developed using ImmPACT DAB. Immunofluorescent analysis was carried out in sections from 3xTg-AD and C57BL6/J mice. Sections were fixed in 4% paraformaldehyde, blocked with 10% horse serum with 0.3% triton-X, incubated with primary antibodies and Alexa fluor secondary antibodies, and mounted using EverBrite™ Medium with DAPI. Primary antibodies include anti-σ₁R (Thermofisher, #42-3300); anti-NeuN (Proteintech, #66836); anti-Olig2 (R&D systems, #AF2418); anti-GFAP (Thermofisher, #14-9892-82); and anti-Iba1 as (Novus, NB100-2833). Whole slide scans were performed using the ZEISS Axioscan 7 scanner.

### PET Brain Imaging in Cynomolgus Macaque

A Focus 220 scanner was utilized to acquire PET imaging data from male macaques (˜9 kg). Arterial blood was collected through a plastic catheter from the femoral artery. Before PET acquisition, a 45-minute transmission scan for attenuation correction was performed. A two-hour dynamic scan was acquired after administration of ˜0.37 GBq radioligand. To quantify the tracer uptake in total and subregions of the brain, images were co-registered to a standardized monkey MRI template using PMOD software 4.3. Predefined brain ROIs from the template were applied to the co-registered PET image to obtain the time-activity curves. The uptake was normalized to body weight and the injected radioactivity to obtain SUV.

### Radiometabolite Studies of Rat Plasma and Brain Samples in SD Rats and Macaque Plasma Samples in Monkeys

To determine the in vivo stability of candidate radioligands, HPLC-radiometabolite analysis was performed in the plasma samples from SD rats and macaques. Additionally, to determine if the identified radiometabolite(s) can enter the brain, radiometabolite analysis in rat brain homogenate samples were performed. For rat, adult male SD rats were intravenously injected with ˜16.8 MBq of radioligand and sacrificed at desired timepoints. Plasma and brain were collected and processed accordingly. For macaques, blood samples were collected from the left ventricle. Sample was loaded on an Oasis HLB online capture column (186001414) and HPLC radiometabolite analysis was performed on an analytical column (Agilent ZORBAX Eclipse XDB-C18).

### Statistical Analysis

All data were analyzed with Prism 10.0 (GraphPad, San Diego, CA). For binding data, the inhibition constant (Ki) was determined by a nonlinear regression analysis of a one-site competitive binding model. Two-way ANOVA and student t-test were used to determine the difference among sample groups. A P value ≤0.05 was considered to be statistically significant.

### Example 2—Synthesis of Sigma-1 Receptor Ligands

### Synthesis of σ₁R Ligands:

Racemic σ₁R compounds, (±)-13, (±)-14, (±)-15, and (±)-21 along with their enantiomers, and hydroxy precursors (±)-11 for (±)-[¹⁸F]13, (−)-11 for (−)-[¹⁸F]13, (+)-11 for (+)-[¹⁸F]13, (−)-12 for (−)-14 and (−)-15, (−)-20 for (−)-21 were synthesized according to reported procedures (FIG. 7, FIG. 8). The strategies are 1) to examine the impact of changing of fluorobenzyl group on TZ3108 to fluoroethoxy benzyl group on compound 14; 2) to examine the impact of changing 1,4′-bipiperidin on TZ3108 and compound 14 to 1,3′-bipiperidin on compound 13; 3) to examine the impact of changing of fluorobenzyl group on TZ3108 to fluoroethoxy benzyl group on compound 21; 4) to examine the impact of changing fluorobenzaldehyde on TZ3108 to fluorine on the fluorobenzene on compound 15 ([¹⁸F]TZ3108B). The chiral resolution of the racemic compounds (±)-13, (±)-14, (±)-21 was achieved through normal-phase high-performance liquid chromatography (HPLC) separation⁷. The absolute configuration and NMR data of these molecules, providing structural evidence, were confirmed in our earlier publications. The HPLC mobile phase and column details the unknown compounds, along with the enantiomeric excess (ee) of chiral isomers determined by normal phase HPLC (FIG. 9, FIG. 10).

((3′R,4′R)-1′-(4-(2-Fluoroethoxy)benzyl)-4′-hydroxy-[1,3′-bipiperidin]-4-yl)(4-fluorophenyl)-methanone [(−)-13] or [(−)-TZ9580]: According to the reference method, a 15 mL oven-dried pressure tube was charged with the phenol precursor (−)-11 (prepared according to the reference method⁷) (80 mg, 0.1941 mmol), 1-Fluoro-2-iodoethane (16 μL, 0.1941 mmol), potassium carbonate (40.2 mg, 0.2911 mmol), and DMF (3 mL). The mixture was heated at 120° C. for 12 hours. After the reaction, the mixture was cooled to room temperature, and the crude product was washed with water (3×10 mL) and ethyl acetate (3×5 mL). It was then dried over MgSO₄ and concentrated under reduced pressure. The crude was purified by flash chromatography (hexane/Ethyl acetate 20:80) to afford the (−)-TZ9580 (71 mg, 80%) as a brown solid; mp 112-114° C. ¹H NMR (400 MHz, CDCl₃) δ 7.94 (dd, J=7.6, 5.7 Hz, 2H), 7.25 (d, J=8.8 Hz, 2H), 7.18-7.08 (m, 2H), 6.90 (d, J=8.0 Hz, 2H), 4.87-4.78 (m, 1H), 4.75-4.65 (m, 1H), 4.30-4.22 (m, 1H), 4.22-4.15 (m, 1H), 3.52 (s, 2H), 3.50-3.41 (m, 1H), 3.27-3.14 (m, 1H), 3.02 (d, J=10.7 Hz, 2H), 2.92-2.80 (m, 2H), 2.79-2.69 (m, 1H), 2.60 (t, J=9.3 Hz, 1H), 2.30 (t, J=11.0 Hz, 1H), 1.09-1.56 (m, 3H), 1.90-1.56 (m, 5H); ¹³C NMR (100 MHz, CDCl₃) δ 200.97, 165.80 (d, J_{CF}=254.9 Hz), 157.86, 132.42 (d, J_{CF}=3.1 Hz), 131.04, 130.94, 130.52, 115.94 (d, J_{CF}=21.8 Hz), 114.58, 82.10 (d, J_{CF}=170.7 Hz), 67.86, 67.39 (d, J_{CF}=4.4 Hz), 67.17, 62.32, 52.09, 51.48, 49.95, 45.67, 43.75, 32.10, 29.62, 29.30; ¹⁹F NMR (376 MHz, CDCl₃) δ −105.32; HRMS (ESI): m/z calculated for C₂₆H₃₃F₂N₂O₃ [M+H]⁺: 459.2454. Found: 459.2458. The optical rotation of (−)-TZ9580 was [α]_{D}²⁰=−12.3° (3.0 mg/mL in MeOH). The isomer (+)-TZ9580 was confirmed by ESI-MS. Calcd. For C₂₆H₃₃F₂N₂O₃ [M+H]⁺ m/z 459.24; found m/z 459.56. The optical rotation of (+)-TZ9580 was [α]_{D}²⁰=+11.8° (6.7 mg/mL in MeOH).

((3′R,4′R)-1′-(4-(2-Fluoroethoxy)benzyl)-3′-hydroxy-[1,4′-bipiperidin]-4-yl)(4-fluorophenyl)-methanone[(−)14] or [(−)-TZ96110]: According to the reference method, a 15 mL oven-dried pressure tube was charged with the phenol precursor (−)-12 (prepared according to the reference method⁷) (80 mg, 0.1941 mmol), 1-Fluoro-2-iodoethane (16 μL, 0.1941 mmol), potassium carbonate (40.2 mg, 0.2911 mmol), and DMF (3 mL). The mixture was heated at 120° C. for 12 hours. After the reaction, the mixture was cooled to room temperature, and the crude product was washed with water (3×10 mL) and ethyl acetate (3×5 mL). It was then dried over MgSO₄ and concentrated under reduced pressure. The crude was purified by flash chromatography (hexane/Ethyl acetate 20:80) to afford the (−)TZ96110 (69 mg, 76%) as a brown solid; mp 104-106° C. ¹H NMR (400 MHz, CDCl₃) δ 7.95 (dd, J=8.7, 5.4 Hz, 2H), 7.21 (d, J=8.5 Hz, 2H), 7.17-7.08 (m, 2H), 6.87 (d, J=8.5 Hz, 2H), 4.85-4.79 (m, 1H), 4.76-4.66 (m, 1H), 4.31-4.22 (m, 1H), 4.21-4.12 (m, 1H), 3.63 (s, 1H), 3.51 (s, 2H), 3.37-3.16 (m, 2H), 2.99 (dd, J=17.7, 12.8 Hz, 2H), 2.88-2.62 (m, 2H), 2.46-2.19 (m, 2H), 2.12-1.83 (m, 5H), 1.75 (d, J=10.5 Hz, 2H), 1.67-1.45 (m, 2H); ¹³C NMR (100 MHz, CDCl₃) δ 200.84, 165.66 (d, J_{CF}=254.9 Hz), 157.70, 132.25 (d, J_{CF}=2.7 Hz), 130.91, 130.81, 130.53, 115.81 (d, J_{CF}=21.8 Hz), 114.37, 81.94 (d, J_{CF}=170.7 Hz), 69.40, 67.12 (d, J_{CF}=20.5 Hz), 65.85, 61.86, 58.52, 52.64, 45.49, 29.00, 28.96, 21.56, 29.71, 29.41; ¹⁹F NMR (376 MHz, CDCl₃) δ −75.57; HRMS (ESI): m/z calculated for C₂₆H₃₃F₂N₂O₃ [M+H]⁺: 459.2454. Found: 459.2459. The optical rotation of (−)-TZ96110 was [α]_{D}²⁰=−31.4° (0.7 mg/mL in MeOH). The isomer (+)-TZ96110 was confirmed by ESI-MS. Calcd. For C₂₆H₃₃F₂N₂O₃ [M+H]⁺ m/z 459.24; found m/z 459.58. The optical rotation of (+)-TZ96110 was [α]_{D}²⁰=+33.3° (0.9 mg/mL in MeOH).

((3′R,4′R)-1′-(4-Fluorobenzyl)-3′-hydroxy-[1,4′-bipiperidin]-4-yl)(4-(2-fluoroethoxy)-phenyl)-methanone [(−)-21] or [(−)-TZ1064]: According to the reference method¹¹, a 15 mL oven-dried pressure tube was charged with the phenol precursor (−)-20 (prepared according to the reference method^{8,9}) (70 mg, 0.169 mmol), 1-Fluoro-2-iodoethane (40 μL, 0.507 mmol), potassium carbonate (35 mg, 0.250 mmol), and DMF (3 mL). The mixture was heated at 120° C. for 12 hours. After the reaction, the mixture was cooled to room temperature, and the crude product was washed with water (3×10 mL) and ethyl acetate (3×5 mL). It was then dried over MgSO₄ and concentrated under reduced pressure. The crude was purified by flash chromatography (Ethyl acetate:MeOH 99:1) to afford the (−)-TZ1064 (60 mg, 77%) as a pale brown solid; mp 126-128° C. ¹H NMR (400 MHz, CDCl₃) δ 7.92 (d, J=8.8 Hz, 2H), 7.27-7.23 (m, 2H), 7.02-6.95 (m, 4H), 4.86-4.81 (m, 1H), 4.74-4.70 (m, 1H), 4.33-4.29 (m, 1H), 4.26-4.22 (m, 1H), 3.59 (td, J=9.8, 4.6 Hz, 1H), 3.54-3.46 (m, 2H), 3.23-3.18 (m, 2H), 2.98 (d, J=11.4 Hz, 1H), 2.92 (d, J=10.8 Hz, 1H), 2.78-2.70 (m, 2H), 2.31-2.21 (m, 2H), 1.98 (t, J=10.5 Hz, 1H), 1.89-1.84 (m, 4H), 1.78-1.71 (m, 2H), 1.60-1.52 (m, 1H); ¹³C NMR (100 MHz, CDCl₃) δ 201.02, 163.20, 162.12, 133.71, 130.96, 130.53 (d, J 23.0 Hz), 129.40, 115.29 (d, J 27.3 Hz), 114.66, 81.60 9 (d, J 171.3 Hz), 69.56, 69.40, 67.15 (d, J 20.4 Hz), 66.13, 65.75, 61.83, 53.30, 52.63, 43.36, 29.43, 29.28, 21.55; HRMS (ESI): m/z calculated for C₂₆H₃₃F₂N₂O₃ [M+H]⁺: 459.2454. Found: 459.2458. The optical rotation of (−)-TZ1064 was [α]_{D}²⁰=−27.7° (0.65 mg/mL in MeOH). The isomer (+)-TZ1064 was confirmed by ESI-MS. Calcd. For C₂₆H₃₃F₂N₂O₃ [M+H]⁺ m/z 459.24; found m/z 459.41. The optical rotation of (+)-TZ1064 was [α]_{D}²⁰=+30° (0.7 mg/mL in MeOH).

### Radiochemistry

Two F-18 radiochemistry strategies were employed to radiosynthesize these new F-18 radioligands. The radiosynthesis of (±)-[¹⁸F]13, (−)-[¹⁸F]13, (+)-[¹⁸F]13, (−)-[¹⁸F]14 and (−)-[¹⁸F]21 was achieved using a two-step procedure: 1) nucleophilic substitution of the ditosylate precursor 17 with dried K[¹⁸F]/F⁻; 2) O-alkylation of the phenol precursor with 2-[¹⁸F]fluoroethyl tosylate [¹⁸F]18 in the presence of cesium carbonate (Cs₂CO₃). Meanwhile, (−)-[¹⁸F]15 was radiosynthesized using an innovative ruthenium-mediated radiofluorination ([¹⁸F]/F⁻) chemistry on an aromatic phenol precursor (−)-12. The intended products were isolated from the precursors and side products using a semi-preparative reverse-phase HPLC column under optimized conditions. During the HPLC purification of radiotracers, we optimized the mobile phase conditions for each radioligand (20-40% acetonitrile in 0.1M ammonium formate buffer, pH ˜4.5) to allow the radioactive product peak to elute 10-15 min post its hydroxy precursor(s) to make sure a good separation. This allowed us to achieve high chemical purity without contaminating respective hydroxy precursor(s). The [¹⁸F]fluoride in a 0.2-2.5 mL bolus of [¹⁸O]H₂O and was trapped on a pre-conditioned QMA cartridge (WAT023525, Waters) to remove [¹⁸O]H₂O and other aqueous impurities. [¹⁸F]Fluoride was eluted into the reaction vessel using aqueous potassium carbonate solution (3.0 mg mL⁻¹).

1-[¹⁸F]Fluoro-2-tosyloxyethane ([¹⁸F]18): [¹⁸F]KF (˜7.4 GBq) aqueous solution was added to a reaction vessel containing Kryptofix 2.2.2 (7-9 mg). Acetonitrile (3×1.0 mL) was added to the mixture to azeotropically remove water at ˜100° C. with nitrogen gas bubbling through the mixture. After the water was removed, 1,2-ethylene ditosylate (11-13 mg) was dissolved in acetonitrile (300 μL) and transferred to the reaction vessel containing [¹⁸F]fluoride/Kryptofix/K₂CO₃. The reaction vessel was capped, vortexed, and heated at 100° C. for 10 min in an oil bath, with agitation performed five times during this period. The reaction was then diluted in 3.0 mL of HPLC mobile phase (50% acetonitrile in 0.1 M ammonium formate buffer, pH ˜6.5). This crude product was purified with HPLC (Phenomenex Luna column (250×9.6 mm, 5 μm) semipreparative column, mobile phase: 50% acetonitrile in 0.1 M ammonium formate buffer, pH 6.5, flow rate: 4.0 mL/min, UV detector set: 254 nm) by collecting the portion with a retention time of 13-15 min according to the radioactive signal. The product eluted from the HPLC was diluted with 50 mL of sterile water and passed through a C-18 Sep-Pak Plus cartridge, where it remained on the cartridge. Ether (2.5 mL) was used to elute the trapped 1-[¹⁸F]fluoro-2-tosyloxyethane off of the Sep-Pak to afford 3.7-4.02 GBq of product with 65-70% radiochemical yield (decay corrected to the end of synthesis (EOS)). The synthesis of 1-[¹⁸F]fluoro-2-tosyloxyethane took about 40 min.

(±)-[¹⁸F]13, (−)-[¹⁸F]13, and (+)-[¹⁸F]13: The upper ether layer containing 1-[¹⁸F]fluoro-2-tosyloxyethane ([¹⁸F]18) was passed through a set of two Sep-Pak Plus dry cartridges, transferred into a reaction vessel, and evaporated under a nitrogen stream at 25° C. The phenolic precursor (1.5-2 mg) and Cs₂CO₃ (4 mg) were added in anhydrous DMSO (200 μL) to the dried vial. The vial was sealed and heated again at 110-120° C. for 10 minutes in an oil bath. Subsequently, the reaction solution was diluted with 3.0 mL of HPLC mobile phase (38% acetonitrile in 0.1 M ammonium formate buffer, pH ˜4.5) and loaded onto a C₁₈ column (Phenomenex Luna, 250×9.6 mm, 5 μm). The product was eluted using the same HPLC mobile phase at a flow rate of 4.0 mL/min, with UV detection at 254 nm. A 100 mL glass vial containing 50 mL of sterile water was used to collect the radioactive product, which was eluted from 21 to 23 minutes. The diluted product was subsequently passed through a C₁₈ Sep-Pak Plus cartridge (Part No. WAT020515) with nitrogen gas assistance. Finally, the trapped product was eluted using 10% ethanol in 0.9% saline to formulate the injection dose for quality control analysis and animal studies. This process obtained 800-870 MBq (±)-[¹⁸F]13, (−)-[¹⁸F]13, and (+)-[¹⁸F]13, with 23-25% radiochemical yield (decay corrected to EOS). To check the quality of (±)-[¹⁸F]13, (−)-[¹⁸F]13, and (+)-[¹⁸F]13 an aliquot of the sample was co-injected with non-radiolabeled standard 13 onto an analytical HPLC system equipped with a Phenomenex SB-C18 analytical column 250×4.6 mm, 5 μm using an isocratic elution profile (mobile phase: (48% acetonitrile in 0.1 M ammonium formate buffer, pH ˜4.5, flow rate: 1.0 mL/min, UV detector set: 254 nm). The final product had a radiochemical purity of >95% and molar activity of <44.3 MBq/nmol (decay corrected to EOS). The synthesis of (±)-[¹⁸F]13, (−)-[¹⁸F]13, and (+)-[¹⁸F]13 took about 70 min, and the entire two-step radiolabeling took about 2 h.

(−)-[¹⁸F]14: The upper ether layer containing 1-[¹⁸F]fluoro-2-tosyloxyethane ([¹⁸F]18) was passed through a set of two Sep-Pak Plus dry cartridges, transferred into a reaction vessel, and evaporated under a nitrogen stream at 25° C. The phenolic precursor (1.5-2 mg) and Cs₂CO₃ (4 mg) were added in anhydrous DMSO (200 μL) to the dried vial. The vial was sealed and heated again at 110-120° C. for 10 minutes in an oil bath. Subsequently, the reaction solution was diluted with 3.0 mL of HPLC mobile phase (25% acetonitrile in 0.1 M ammonium formate buffer, pH ˜4.5) and loaded onto a C18 column (Agilent, 250×9.6 mm, 5 μm). The product was eluted using the same HPLC mobile phase at a flow rate of 4.0 mL/min, with UV detection at 254 nm. A 100 mL glass vial containing 50 mL of sterile water was used to collect the radioactive product, which was eluted from 20 to 22 minutes. The diluted product was subsequently passed through a C18 Sep-Pak Plus cartridge (Part No. WAT020515) with nitrogen gas assistance. Finally, the trapped product was eluted using 10% ethanol in 0.9% saline to formulate the injection dose for quality control analysis and animal studies. This process obtains 870-940 MBq (−)-[¹⁸F]14 with a 25-27% radiochemical yield (decay corrected to EOS). To check the quality of (−)-[¹⁸F]14 an aliquot of the sample, was co-injected with non-radiolabeled standard 14 onto an analytical HPLC system equipped with a (Agilent SB-C18, 250×4.6 mm, 5 μm) analytical column using an isocratic elution profile (mobile phase: (45% acetonitrile in 0.1 M ammonium formate buffer, pH ˜4.5, flow rate: 1.0 mL/min, UV detector set: 254 nm). The final product had a radiochemical purity of >95% and molar activity of <58.1 MBq/nmol (decay corrected to EOS). The synthesis of (−)-[¹⁸F]14 took about 70 min, and the entire two-step radiolabeling took about 2 h.

(−)-[¹⁸F]21: The upper ether layer from the earlier step contains 1-[¹⁸F]fluoro-2-tosyloxyethane ([¹⁸F]18) was passed through a set of two Sep-Pak Plus dry cartridges, transferred into a reaction vessel, and evaporated under a nitrogen stream at 25° C. The phenolic precursor (1.5-2 mg) and Cs₂CO₃ (4 mg) were added in anhydrous DMSO (200 μL) to the dried vial. The vial was sealed and heated again at 110-120° C. for 10 minutes in an oil bath. Subsequently, the reaction solution was diluted with 3.0 mL of HPLC mobile phase (30% acetonitrile in 0.1 M ammonium formate buffer, pH ˜4.5) and loaded onto a C18 column (Agilent, 250×9.6 mm, 5 μm). The product was eluted using the same HPLC mobile phase at a flow rate of 4.0 mL/min, with UV detection at 254 nm. A 100 mL glass vial containing 50 mL of sterile water was used to collect the radioactive product, which was eluted from 17 to 19 minutes. The diluted product was subsequently passed through a C18 Sep-Pak Plus cartridge (Part No. WAT020515) with nitrogen gas assistance. Finally, the trapped product was eluted using 10% ethanol and 90% saline to formulate the injection dose for quality control analysis and animal studies. This process obtains 925-1110 MBq (−)-[¹⁸F]21 with a 35-40% radiochemical yield (decay corrected to EOS). To check the quality of (−)-[¹⁸F]21 an aliquot of the sample, was co-injected with non-radiolabeled standard 21 onto an analytical HPLC system equipped with a (Agilent SB-C18, 250×4.6 mm, 5 μm) analytical column using an isocratic elution profile (mobile phase: (40% acetonitrile in 0.1 M ammonium formate buffer, pH ˜4.5, flow rate: 1.0 mL/min, UV detector set: 254 nm). The final product had a radiochemical purity of >99% and molar activity of >40 GBq/μmol (decay corrected to EOS). The synthesis of (−)-[¹⁸F]21 took about 60 min, and the entire two-step radiolabeling took about 2 h.

(−)-[¹⁸F]15: A bolus of aqueous [¹⁸F]fluoride (˜200 mCi, ˜7.4 GBq) was transferred into a glass V-vial containing aqueous potassium carbonate solution (40 μL, 45 mg/mL). Then 1.5 mL of CH₃CN solution of Kryptofix 222 (6-7 mg) was added to the conical glass vial. The solution was dried by three cycles of azeotropic evaporation with additional CH₃CN (2×1.0 mL) under a gentle stream of nitrogen gas at 100° C. Phenol precursor (−)-12 (4.8 μmol, 2.0 mg) and ruthenium complex (4.5 mg, 14.56 μmol, 3 equiv.) were dissolved in ethanol (50 μL) in a V glass vial and heated at 85° C. for 30 min. The vial was removed from the heating bath and allowed to stand for 3 min at 23° C. To the vial, imidazolium chloride (N,N′-bis(2,6-diisopropylphenyl)-2-chloroimidazolium chloride (6.7 mg, 14.56 μmol, 3.0 equiv.), 175 μL of MeCN and 175 μL of DMSO were added. The resulting solution was drawn into a 1.0 mL polypropylene syringe and transferred into the reaction vial containing the [¹⁸F]K¹⁸F. The reaction vial, which contained 400 μL of the reaction mixture was sealed with a Teflon-lined cap and was heated at 160° C. for 30 min. After cooling by water-bath, the reaction mixture was analyzed by radio-TLC before being quenched by 2 mL of HPLC mobile phase (20% acetonitrile in 0.1 M ammonium formate buffer, pH 4.5). The solution was loaded onto a semi-preparative HPLC system for purification. The HPLC system contains a 5 mL injection loop, a Phenomenex Luna column (250×9.4 mm, 5μ), a UV detector at 254 nm, and a radioactivity detector. With mobile phase mentioned above as the eluent with a flow rate of 4 mL/min, the retention time of the product was 34-36 min. The product collection was diluted using sterile water (˜50 mL) and then passed through a C18 Sep-Pak Plus cartridge. The trapped product was eluted using ethanol (0.6 mL), followed by 0.9% saline (5.4 mL). After sterile filtration into a glass vial, (−)-[¹⁸F]15 was ready for quality control (QC) analysis and animal studies. To check the quality of (−)-[¹⁸F]15, an aliquot of the sample was co-injected with the non-radiolabeled standard (−)-15 sample solution onto an analytical HPLC system equipped with a Phenomenex Luna SB-C18 column (250×4.6 mm, 5μ) and UV absorbance at 254 nm; the mobile phase consisted of acetonitrile/0.1 M ammonium formate buffer (40/60, v/v, pH 4.5). Under these conditions, the retention time of (−)-[¹⁸F]15 was 4.6 min at a flow rate of 1.0 mL/min. The decay-corrected radiochemical yield of (−)-[¹⁸F]15 was 28 to 30% with good radiochemical purity (>95%) and molar activity (>47 GBq/μmol, decay corrected to EOS). The synthesis of (−)-[¹⁸F]15 took about 120 min including the K[¹⁸F]/F⁻ drying.

### Ex Vivo Biodistribution of Lead σ1R Radioligands in SD Rats.

Several σ₁R radioligands have been previously tested in humans and have difficulties reaching equilibrium within hours of the scan. Notably, we have previously shown that our lead radioligand (−)-[¹⁸F]TZ3108 could reach equilibrium and wash out from the nonhuman primate brain, and (−)-enantiomer is more promising than (+) enantiomer for these structural analogues^{4,11}. Therefore, to further understand the biochemical properties of our lead and new σ₁R radioligands in vivo, particularly the impact of structure differences on the radioligand dynamics in brain and other tissues, we next performed an ex vivo biodistribution analysis of (−)-[¹⁸F]13 on adult male SD rats and compared with our previously published ex vivo biodistribution data of (−)-[¹⁸F]TZ3108 (FIG. 1B, Table 4). As expected, similar to (−)-[¹⁸F]TZ3108, (−)-[¹⁸F]13 entered the brain very well with a % ID/g value of 0.98±0.11 at 5 min compared to (−)-[¹⁸F]TZ3108 with uptake of 1.29±0.06 at 5 min, demonstrated a good BBB permeability of these structural analogues. Interestingly, (−)-[¹⁸F]13 showed a much faster brain washout kinetics with brain uptake values of 0.34±0.1, 0.30±0.03, and 0.26±0.01 at 30, 60, and 120 min respectively, compared to (−)-[¹⁸F]TZ3108 with brain uptake values of 1.06±0.13, 0.86±0.09, and 0.80±0.13 at 30, 60, and 120 min. In addition to the brain, the uptake of (−)-[¹⁸F]13 in other organs was high but slightly lower than (−)-[¹⁸F]TZ3108. For example, (−)-[¹⁸F]13 showed high uptakes in lung, kidney, liver, spleen, and pancreas with % ID/g values of 7.69±0.47, 2.80±0.44, 1.89±0.32, 1.87±0.43, and 1.39±0.25 at 5 min respectively, and then quickly washed out from these organs. Whereas (−)-[¹⁸F]TZ3108 had % ID/g values of 12.29±0.86, 4.75±0.39, 1.62±0.42, 2.52±0.51, and 1.74±0.69 in these organs at 5 min. Notably, (−)-[¹⁸F]TZ3108 was accumulated in liver with increased uptake values of 1.62±0.42, 2.76±0.40, and 2.84±1.49 at 5, 30, and 60 min and started to decrease at 120 min with an uptake of 2.81±0.87. Similarly, accumulation and slow trend of washout were also found in spleen, pancreas, and kidney. In contrast, (−)-[¹⁸F]13 had high uptake at 5 min and then quickly washed out from these organs, suggesting a slower kinetics of (−)-[¹⁸F]TZ3108 in metabolic organs compared to (−)-[¹⁸F]13. Both radiotracers had very low uptake in fat and muscle, consistent to lower expression of σ₁R in fat and muscle, suggesting that both radiotracers have in vivo specificity toward σ₁R in peripheral tissues. Moreover, low uptake was observed in the bone with no trend of increase indicating no defluorination for these ¹⁸F radiotracers. In summary, our ex vivo biodistribution data for (−)-[¹⁸F]13 and (−)-[¹⁸F]TZ3108 showed a high brain uptake, particularly with a fast brain washout dynamic in rats, suggesting our radioligand has an advantageous promise to overcome issues with previous σ₁R radioligands that cannot reach equilibrium within hours post injection.

### PET Imaging and In Vivo Specificity of Candidate σ₁R Radioligands in Mouse Brain.

To characterize the brain uptake, its brain washout kinetics, and specificity of our candidate radioligands in vivo, we next performed PET imaging studies at baseline and pretreatment with known σ₁R ligands in adult male CD-1 mice. In general, all candidate radioligands include (−)-[¹⁸F]13 (FIG. 2A, FIG. 2B), (−)-[¹⁸F]14 (FIG. 2C, FIG. 2D), (−)-[¹⁸F]TZ3108 (FIG. 2E, FIG. 2F), and (−)-[¹⁸F]15 (or (−)-[¹⁸F]TZ3108B) (FIG. 2G, FIG. 2H) penetrated the BBB well with high brain uptake in mouse brains. More importantly, all four radiotracers showed a good brain washout within 60 min post injection. (−)-[¹⁸F]13 quickly entered the brain and reached a peak SUV of ˜2.4 at 3 min post-injection and then quickly washed out from the brain with SUVs of 2.02, 1.53, 0.91, and 0.65 at 10-, 20-, 40-, and 60-min post-injection. Particularly, pretreatment with well-known σ₁R antagonist Haloperidol at 2 mg/kg 5 min prior to the radiotracer injection significantly reduced the brain uptake of (−)-[¹⁸F]13, the average SUV reduce from 20 to 40 min was ˜26.1% for pretreatment using Haloperidol with a p-value of 0.0062. Moreover, pretreatment with cold (−)-TZ3108 at 2 mg/kg also reduced 17.9% brain uptake of (−)-[¹⁸F]13 with a p-value of <0.0356 (FIG. 2A, FIG. 2B, FIG. 2J). In contrast, (−)-[¹⁸F]14 showed a relatively slower brain washout pharmacokinetics with a slightly higher peak of brain uptake (SUV) of ˜3.39 at 2 min, and SUVs of 3.07, 2.71, 2.10, and 1.68 at 10, 20, 40 and 60 min respectively. Pre-injection of Haloperidol and (−)-TZ3108 showed a 27.6% and 39.7% reduction in brain uptake of (−)-[¹⁸F]14 with p values of 0.0001 and 0.0003 respectively (FIG. 2C, FIG. 2D, FIG. 2K). Compared to (−)-[¹⁸F]13 and (−)-[¹⁸F]14, (−)-[¹⁸F]TZ3108 and (−)-[¹⁸F]15 showed a slower rat brain washout pharmacokinetics but with a higher brain uptake. As expected, both (−)-[¹⁸F]TZ3108 and (−)-[¹⁸F]15 had nearly identical mouse brain washout pharmacokinetics, indicating that different labeling sites did not cause significant brain washout pharmacokinetics in the mouse. (−)-[¹⁸F]TZ3108 and (−)-[¹⁸F]15 entered the brain quickly with a peak brain uptake (SUV) of 4.35 at 2 min and 4.39 at 3 min respectively. (−)-[¹⁸F]TZ3108 then gradually washed out from the mouse brain with SUVs of 3.99, 3.42, 2.75, and 2.47 at 10, 20, 40, and 60 min post-injection (FIG. 2E, FIG. 2F), whereas (−)-[¹⁸F]15 had SUVs of 4.26, 3.74, 3.02, and 2.67 at 10-, 20-, 40-, and 60-min post-injection (FIG. 2G, FIG. 2H). Pretreatment with Haloperidol and cold (−)-TZ3108 caused a significant reduction of (−)-[¹⁸F]TZ3108 brain uptake in mouse, the average SUV from 40 to 60 min showed a 42.3% reduction in mouse pretreatment using Haloperidol with a p-value of 0.0003 and a 62.9% reduction in mouse pretreatment using (−)-TZ3108 with a p-value of <0.0001 (FIG. 2E, FIG. 2F, FIG. 2L). Similarly, pretreatment with Haloperidol significantly reduced the brain uptake of (−)-[¹⁸F]15 by 44.0% from 40 to 60 min (p=0.0207); and pretreatment using cold (−)-TZ3108 reduced 60.9% average brain uptake (SUV) from 40 to 60 min in mouse with p value of 0.0069 (FIG. 2G, FIG. 2H, FIG. 2M). In summary, PET studies in mouse brains showed all four sigma-1 radioligands have very good brain uptake and remarkable in vivo specificity. Particularly, all four radioligands showed a clear trend of brain washout within 60 min post injection, indicating a clinically favorable brain washout pharmacokinetics.

PET Imaging of Lead σ1R Radioligands in 3xTg-AD Mouse Brain.

The PET study on normal mice demonstrated high brain uptake and in vivo specificities of lead radiotracers toward σ1R. To test if our σ1R radioligands can quantify of σ1R changes in pathological conditions, particularly in AD, next PET imaging was performed in 11-month-old adult female 3xTg-AD mice and age-matched C57BL/6 mice. Interestingly, the uptake of (−)-[¹⁸F]13 was significantly reduced in the 3xTg-AD mice (Two-way ANOVA: F(1, 320)=155, P<0.0001) with a peak SUV of ˜1.75 compared to that in control C57BL/6 mice with a peak SUV of ˜2.16 (FIG. 3A). The average SUV from 5 to 25 min of 3xTg-AD mice was ˜1.37 compared to the average SUV from 5 to 25 min in C57BL/6 control mice ˜1.65, a 17.0% loss in 3xTg-AD mice (P=0.0215) (FIG. 3B). Further brain regions' analyses showed the uptake of (−)-[¹⁸F]13 was significantly reduced in all analyzed brain regions (Fisher's LSD test, P<0.01 for all regions) (FIG. 3C, FIG. 3K). These results indicated (−)-[¹⁸F]13 is a promising σ1R radioligand and can quantify of σ1R expression changes in AD brain compared to the normal mouse brain. Additionally, our PET imaging studies of (−)-[¹⁸F]14 and (−)-[¹⁸F]TZ3108 in 3xTg-AD mice also showed a significantly reduced brain uptake in the 3xTg-AD mouse compared to C57BL/6 control mouse (Two-way ANOVA: F(1, 240)=164.3, P<0.0001) (FIG. 3D), the average brain uptake SUV from 5 to 25 min was reduced to ˜1.90 in 3xTg-AD mice from ˜2.25 in C57BL/6 control mice, a 15.6% reduction in 3xTg-AD mice compared to control mouse (P=0.0179) (FIG. 3F). Furthermore, the uptake of (−)-[¹⁸F]14 was significantly reduced in major regions except cerebellum (Fisher's LSD multiple comparisons followed by two-way ANOVA, P<0.05), though a clear trend of lower uptake in the cerebellum of 3xTg-AD mice was identified with a P value of 0.0565 (FIG. 3F, FIG. 3L). The uptake of (−)-[¹⁸F]TZ3108 was also significantly reduced in the 3xTg-AD mice (Two-way ANOVA: F(1, 240)=268.1, P<0.0001) with a peak SUV of ˜3.15 compared to that in C57BL/6 control mice with a peak SUV of ˜2.54 at ˜3 min (FIG. 3G). The average SUV from 5 to 25 min of 3xTg-AD mice reduced to ˜2.36 for 5 to 25 min from ˜2.89 C57BL/6 in control mice, a 18.3% reduction in 3xTg-AD mice (P=0.0341) (FIG. 3H) was observed. Furthermore, the uptake of (−)-[¹⁸F]TZ3108 was significantly reduced in major brain regions (Fisher's LSD test, P<0.05 for all regions) (FIG. 3I, FIG. 3M). In summary, PET studies of our lead σ1R radioligands in the brain of 3xTg-AD mouse model of AD showed significant brain uptake reduction for all three sigma-1 radioligands in the 3xTg-AD mice compared to the age-matched control mice, indicating that our radiotracers can detect a reduced expression of σ1R in AD mice compared to control mice. These results demonstrated the radioligands are capable of quantifying expression changes of σ1R in pathological conditions, particularly in AD, and are worth pursuing further evaluations.

In Vitro Immunostaining Characterization of σ1R Expression in Normal and 3xTg-AD Mouse Brain.

Previous studies showed σ₁R is highly expressed in CNS and other organs, however, the expression profile of σ₁R in CNS especially in pathological conditions remains unclear with different expression patterns reported. To confirm our findings from PET studies, we first performed an immunofluorescent analysis of σ₁R in the CNS of normal mouse brain tissues. Our study showed that the expression of σ₁R was high in CNS. High expression of neuronal σ₁R was identified throughout the brain, especially in the cerebral cortex and hippocampus, with nearly all Neuronal Nuclei (NeuN) positive cells colocalized with σ₁R staining (FIG. 21A). σ₁R was also expressed in the oligodendrocyte cells. Oligodendrocytes identified by oligodendrocyte lineage transcription factor 2 (Olig2) showed almost all Olig2+ cells were colocalized with σ₁R staining in both gray matter and white matter of the brain (FIG. 21B). Furthermore, σ₁R was found in at least a portion of astrocytes identified by Glial fibrillary acidic protein (GFAP) staining, indicating σ₁R might be associated with the different states of astrocytes; interestingly, σ₁R was highly colocalized with ventricular membrane and vascular GFAP positive cells (FIG. 21C). Similarly, σ₁R was also found in at least a portion of microglia cells identified by ionized calcium-binding adaptor molecule 1 (Iba-1) staining, indicating σ₁R might be also associated with the different states of microglia cells (FIG. 21D).

### Radiometabolite Analysis of Radiotracers in Macaque and Rat.

To further understand the pharmacokinetics of our lead sigma-1 radiotracers and to examine if radiometabolite can enter the brain and confound the PET quantification, we next performed radiometabolite analysis focusing on our most promising radioligands (−)-[¹⁸F]13 and (−)-[¹⁸F]TZ3108. For (−)-[¹⁸F]13, HPLC plasma radiometabolite analysis showed, at 5 min post-injection, almost all radioactivity was from the parental radiotracer (−)-[¹⁸F]13 with a retention time (RT) of ˜15 min, and then slowly metabolized with the radioactivity percentage from the parental radiotracer as 98.02%, 90.74%, 84.92%, 77.07%, at 74.97% at 5, 15, 30, 60, and 90 min respectively. Only one hydrophilic radiometabolite with a retention time (RT) of ˜7.4 min was identified with 1.98%, 9.26%, 15.08%, 22.93%, and 25.21% of total activity at 5, 15-, 30-, 60-, and 90-min post-injection (FIG. 22A, FIG. 22B). Notably, with ˜75% of total activity remaining as parental radioligand (−)-[¹⁸F]13 at 90 min post-injection, our plasma radiometabolite analysis demonstrates that (−)-[¹⁸F]13 has good in vivo stability and brain washout pharmacokinetics in macaque. Because a small amount of hydrophilic radiometabolite was observed in the macaque plasma samples, to further confirm if this radiometabolite can enter the brain and confound the PET measurement, we performed a HPLC radiometabolite analysis for rat plasma and brain homogenate samples post injection of (−)-[¹⁸F]13. In rat plasma, two hydrophilic radiometabolites with RT of ˜7.4 min and 12.3 min were observed, indicating that the metabolism of radiotracer (−)-[¹⁸F]13 has species difference between rat and macaque. At 5 min, only two radioactivity peaks were observed, 70.83% radioactivity from parental radiotracer (−)-[¹⁸F]13, and 29.17% radioactivity form a radiometabolite (peak 2) with RT of ˜12.3 min; for 90 min rat plasma sample, only 8.69% radioactivity was from parental radiotracer (−)-[¹⁸F]13, a more hydrophilic radiometabolite with 12.02% and RT of ˜7.4 min (peak 1), and 79.29% of radiometabolite peak 2 were observed (FIG. 22E, FIG. 22F). The faster rate of radiometabolism in rat compared to macaque showed the radiotracer has a fast metabolic rate in rat compared to macaque. Importantly, for the rat brain homogenate samples, only the parental radiotracer (−)-[¹⁸F]13 was observed and no radiometabolite was observed for brain samples collected at 5, 30, and 60 min post-injection, indicating the two observed radiometabolites in rat plasma does not enter the brain to confound the PET with (−)-[¹⁸F]13 measurement (FIG. 22G, FIG. 22H), these further indicated our the reduction of radioactivity in the AD brain was results from σ₁R expression decrease in the brain rodent model of AD compared to control mouse. To understand the potential differential pharmacokinetics of (−)-[¹⁸F]13 and its racemic enantiomer (+)-[¹⁸F]13, we also performed a HPLC radiometabolite analysis of (+)-[¹⁸F]13 in NHP plasma samples. Surprisingly, (+)-[¹⁸F]13 had a different and much faster radiometabolism compared to (−)-[¹⁸F]13. Two hydrophilic radiometabolites with RT of ˜7.5 min and ˜8.3 min was observed. A total of 98.51%, 85.43%, 59.97%, 37.71%, and 23.61% radioactivity from parental radiotracer (+)-[¹⁸F]13 was observed for samples collected at 5, 15-, 30-, 60- and 90-min post-injection respectively (FIG. 22C, FIG. 22D). These data indicated a chiral structure-dependent metabolic mechanism of compound (+)-[¹⁸F]13 in vivo. Overall, our radiometabolite analysis of (−)-[¹⁸F]13 in macaque and rat demonstrates that radiotracer (−)-[¹⁸F]13 has a good in vivo stability and has no radiometabolite enter the brain and interfering with PET quantification of σ₁R in the brain.

Similar to radiotracer (−)-[¹⁸F]13, (−)-[¹⁸F]TZ3108 also showed relatively good stability in vivo in macaque. HPLC-radiometabolite showed at 5 min post-injection, no radiometabolite was observed, and parental radiotracer (−)-[¹⁸F]TZ3108 with RT=˜11.6 min remained as 100% of total radioactivity. For plasma samples collected at 15-, 30-, and 60-min post injection of the radiotracer, three radioactive peaks were observed, two hydrophilic radiometabolites were eluted prior to the parental radiotracer peak, radiometabolite 1 with RT of ˜8.1 min and radiometabolite 2 with RT of ˜8.4 min. For plasma samples collected at 15-, 30-, and 60-min post injection, the parent radiotracer (−)-[¹⁸F]TZ3108 was 65.20%, 35.41%, and 16.83% of total radioactivity, whereas radiometabolite 1 was 2.02%, 4.72%, and 9.01% of the total radioactivity, radiometabolite 2 was 32.78%, 59.87%, and 74.17% of the total radioactivity (FIG. 22I, FIG. 22J). To confirm if these two hydrophilic radiometabolites can enter the brain and interfere with PET measurement of σ₁R in CNS, we next performed HPLC radiometabolite analysis of the rat plasma and brain homogenate samples collected and processed post-injection of (−)-[¹⁸F]TZ3108. In rat plasma, (−)-[¹⁸F]TZ3108 was more stable than in macaque plasma post injection, parental (−)-[¹⁸F]TZ3108 retained 77% of total radioactivity at 60 min post injection (FIG. 22M, FIG. 22N). In rat brain homogenate samples, only parental (−)-[¹⁸F]TZ3108 radioactive peak was observed and no radiometabolite was observed, suggesting the hydrophilic radiometabolites observed in rat plasma sample cannot enter the rat brain to impact on the PET measurements of (−)-[¹⁸F]TZ3108 in CNS (FIG. 22O, FIG. 22P).

In summary, radiotracer (−)-[¹⁸F]13 was relatively more stable than (−)-[¹⁸F]TZ3108 in macaque, both of our lead radiotracers showed good in vivo stabilities with no radiometabolites enter the brain to confound the PET measurement in CNS. Radiotracer (−)-[¹⁸F]13 has better in vivo stability in the macaque and only negligible radiometabolite was observed in 90 min plasma sample, our rat metabolite analysis showed that the negligible radiometabolite detected in the rat and macaque plasma samples cannot enter the rat brain to confound PET measurement of σ₁R in the brain. In comparison, (−)-[¹⁸F]TZ3108 exhibits a relatively faster radiometabolism rate than (−)-[¹⁸F]13 although both radiotracers showed no radiometabolite enter into the brain. Our data suggested that both radioligands could be used for PET quantifying σ₁R in the brain, (−)-[¹⁸F]13 is the most promising radiotracer with favorable brain washout pharmacokinetics.

### Example 3—Radiosynthesis and In Vivo Evaluation of Carbon-11 PET Ligands for Imaging the Sigma-1 Receptors in the Brain

To develop radiotracers for PET imaging of σ1 (sigma-1), the following experiments were conducted. Suitable C-11 labeled PET radiotracers for imaging σ1 receptor were identified for transfer to the clinical investigation of patients with AD and other CNS diseases.

Introduction: The sigma-1 receptor (σ1), a multifunctional 25 kDa protein, belongs to a non-opioid receptor family that plays a key role in various physiological and pathological conditions in the central nervous system (CNS). It can be activated by ligands and serves as a chaperone at endoplasmic reticulum membranes. Several neurological disorders, such as Alzheimer's disease (AD), amyotrophic lateral sclerosis, frontotemporal dementia, and Huntington's disease, are linked to σ1 expression and activity. PET Imaging of σ1 could advance our understanding of pathophysiology and assess the therapeutic efficacy of treating diseases targeting the σ1 protein. Patients with AD displayed a decrease in hippocampal σ1 binding sites, and σ1 receptor agonist has potential to ameliorate cognitive deficiencies in AD individuals. We reported our continual efforts on the radiosynthesis and evaluation of a promising C-11 labeled σ1 radioligand (±)-[11C]TZ3114 (Ki-σ1=2.5±0.3 nM) and its minus isomer (−)-[11C]TZ3114 (Ki-σ1=30±2 nM) and plus isomer (+)-[11C]TZ3114 (Ki-σ1=0.82±0.09 nM) for imaging σ1 in central nervous system. The goal of our study is to identify the most suitable C-11 labeled PET radiotracer for imaging σ1 receptor and transferring it to the clinical investigation of patients with AD and other CNS diseases.

Methods: The radiosyntheses of (±)-[11C]TZ3114, (+)-[11C]TZ3114 (−)-[11C]TZ3114 were accomplished by O-[11C]methylation of the corresponding hydroxy precursor with [11C]CH3OTf using NaOH as the base in Dimethylformamide (DMF) heated at 90° C. for 5 min, followed by purification using semi-preparative reverse-phase HPLC. Following an intravenous injection into male non-human primates (NHP), namely macaques, a 2-hour dynamic PET scan was acquired using a preclinical PET/CT scanner, and data was analyzed using PMOD and the standardized uptake values (SUVs) of each tracer in the whole brain were obtained using an in-house macaque brain atlas template. Plasma radiometabolite analysis was also performed for macaque plasma samples collected at 5-, 15-, 30-, and 60 minutes post-injection of each radiotracer.

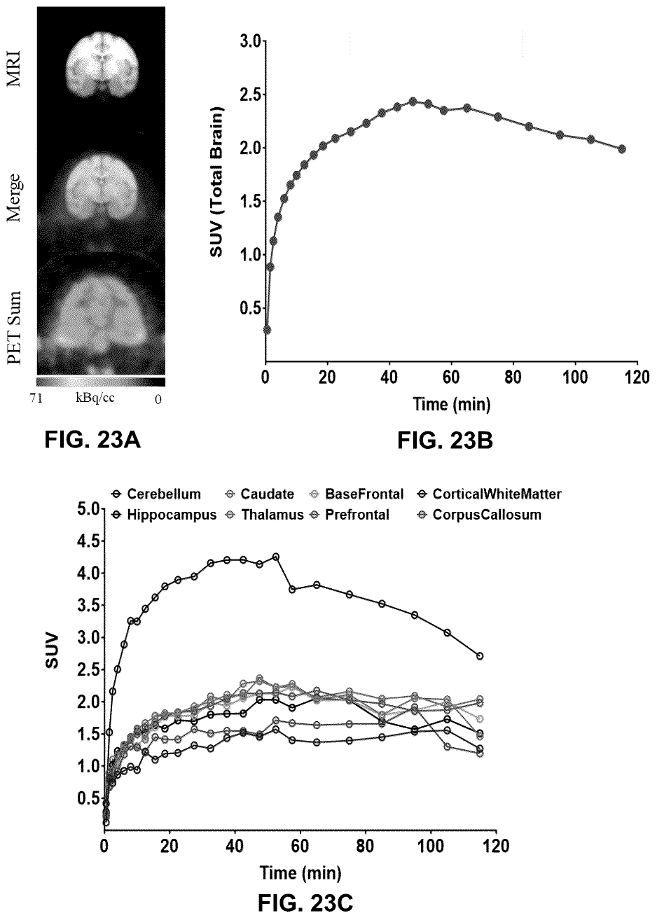

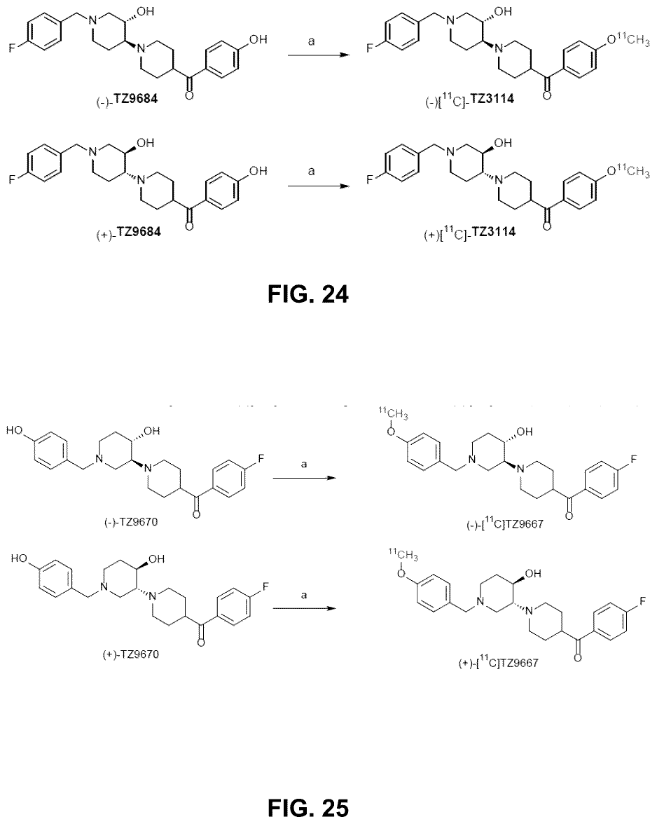

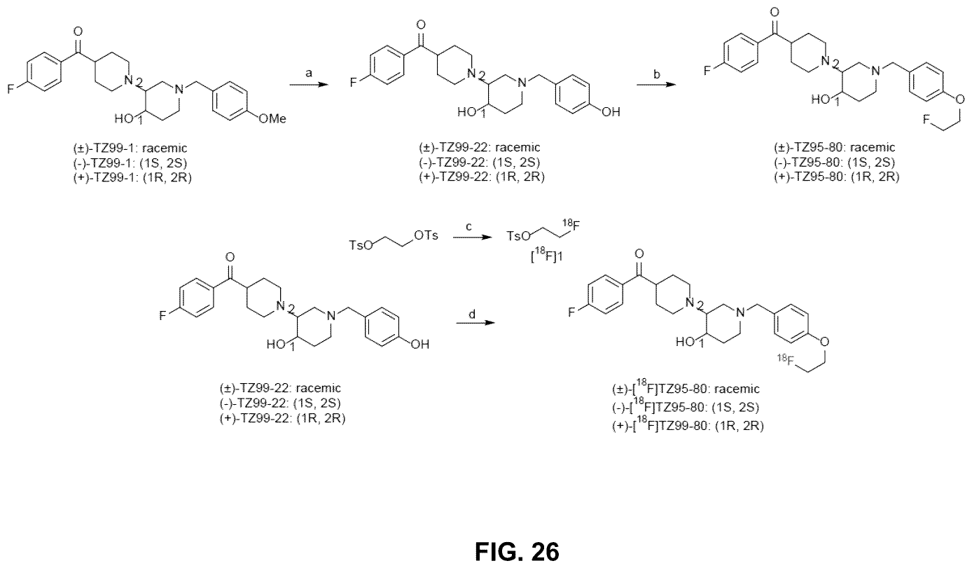

Results: The radiotracers (±)-[11C]TZ3114, (+)-[11C]TZ3114 (−)-[11C]TZ3114 were successfully radiosynthesized with a radiochemical yield of 16-20%, high specific activities of >52 GBq/μmol, and high radiochemical purities of 99%. PET imaging revealed that the SUVs for (+)-[11C]TZ3114 increased gradually and peaked (˜22.0) at 120 min post tracer injection, while the SUVs for (−)-[11C]TZ3114 reached a maximum (˜3.4) at 50 min post-injection, and gradually washed out of the macaque brain. Plasma radiometabolite analysis showed no significant lipophilic radiometabolite was observed for (+)-[11C]TZ3114 and (−)-[11C]TZ3114 at 60 min post-injection (FIG. 23A, FIG. 23B, FIG. 23C).

Conclusion: The radiosyntheses of (±)-[11C]TZ3114, (+)-[11C]TZ3114 and (−)[11C]TZ3114 were achieved with a good radiochemical yield and high purity. The preliminary PET evaluation in NHP revealed that all three radiotracers exhibited high blood-brain barrier permeability, among which (−)-[11C]TZ3114 possessed more favorable washout kinetics in the NHP brain and showed good in vivo stability.

### Example 4—Characterization of Sigma-1 Receptor Tracers

### In Vitro Characterization of Potency and Specificity of TZ95-80 Toward σ1R.

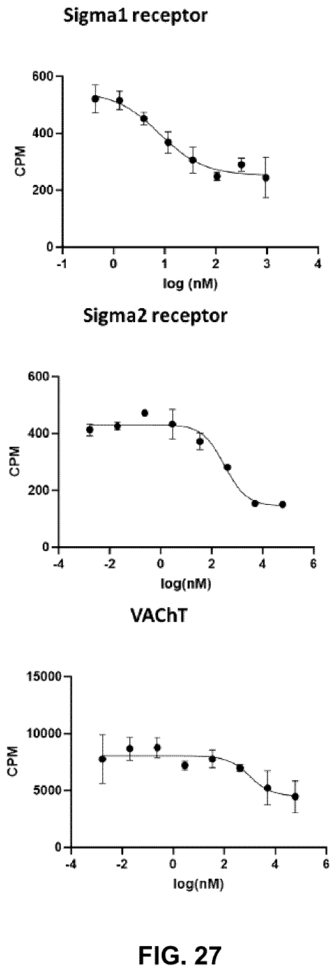

The affinities of TZ9580 binding to σ1R, σ2R, and VAChT were determined to demonstrate the potency and selectivity of our novel σ1R compound TZ9580. In general, TZ9580 showed a high potency toward σ1R, and dozens fold higher toward σ1R and several hundred-fold higher toward VAChT, indicating TZ9580 is a potency and selective compound to σ1R (Table 5, FIG. 27). The (−)-isomer showed an IC50 of 7.9 nM to σ1R, whereas the IC50 of (+)-isomer and racemic mixture are 5.9 nM and 13.3 nM respectively. (−)-isomer showed selectivity to σ1R compared to σ2R and VAChT with an >41-fold and >124-fold higher IC50 values, whereas (+)-isomer showed a >72 fold and >123 fold selectivity toward σ1R and VAChT and the racemic mixture showed a >31 fold and >67 fold selectivity toward σ1R and VAChT. While the overall potency and selectivity amount (−), (+), and racemic mixture are a bit similar, the (+) isomer showed a slightly higher potency toward σ1R and selectivity toward σ2R, like what we previously reported with our other σ1R compounds.

TABLE 5
Binding affinities of TZ9580 to σ1R, S2R, and VAChT.

| | IC50 (nM) | | |
|---|---|---|---|
| Compounds | σ1R | σ2R | VAChT |
| (±)TZ9580 | 13.3 ± 8.8 | 409.4 ± 144.9 | 882.8 ± 130.2 |
| (+)TZ9580 | 5.9 ± 0.9 | 430.5 ± 108.7 | 731.3 ± 24.8 |
| (−)TZ9580 | 7.9 ± 1.4 | 328.3 ± 131.5 | 984.7 ± 50.8 |

### Ex Vivo Biodistribution of (−)[18F]TZ95-80 in SD Rats:

The ex vivo biodistribution analysis of (−)[18F]TZ9580 was performed in male Sprague Dawley (SD) rats to evaluate the tracer dynamic and uptake of (−)[18F]TZ9580 in different organs. The uptake of (−)[18F]TZ9580 was high in the lung, liver, spleen, kidney, pancreas, and brain with a % ID/g value of 7.69, 1.89, 1.87, 2.80, 1.39, and 0.98 respectively at 5 min post-injection. (−)[18F]TZ9580 entered these organs quickly and then washed out from these organs. For example, the uptake of (−)[18F]TZ9580 in the lung was 2.16, 1.61, and 0.88 at 30 min, 60 min, and 120 min post-injection of tracers. Additionally, the uptake of (−)[18F]TZ9580 was low in the bone, indicating no defluorination of 18F and accumulation of bone uptake of (−)[18F]TZ9580 (Table 6).

To test the specificity of (−)[18F]TZ9580, we next examine the biodistribution of (−)[18F]TZ9580 with preblocking with σ1R compounds including the well-known σ1R ligand (+)pentazocine at 2 mg/kg and our previous published compound (−)TZ3108 at 2 mg/kg at 60 min post injection. As expected, both (+)pentazocine and (−)TZ3108 showed a significant blocking effect. Two-way ANOVA showed a F(1,60)=8.37 with a P value of 0.0053 between the treatment and (+) pentazocine-treated group. The uptake of (−)[18F]TZ9580 in lung, liver, spleen, kidney, and pancreas were all reduced after the treatment with (+)pentazocine with a P value of <0.0001 and 0.0058 in lung and kidney using Fisher's LSD test. Pretreatment with (−)TZ3108 showed more blocking effect compared to (+)pentazocine with a F(1,60)=20.97 and a P<0.0001 using Two-way ANOVA, and the uptake in lung, liver, spleen, and kidney were significantly reduced with a P value of 0.0223, <0.0001, 0.0299, and 0.0273 respectively using Fisher's LSD test. The blocking effect from both (+)pentazocine and (−)TZ3108 indicated (−)[18F]TZ9580 binds to σ1R (Table 7).

TABLE 6
Biodistribution of (−)[18F]TZ9580 in male Sprague Dawley
rats (mean ± SD, % ID/g, n = 4).

| Organs | 5 min | 30 min | 60 min | 120 min |
|---|---|---|---|---|
| Blood | 0.15 ± 0.02 | 0.21 ± 0.07 | 0.35 ± 0.02 | 0.35 ± 0.04 |
| Lung | 7.69 ± 0.47 | 2.16 ± 0.54 | 1.61 ± 0.2 | 0.88 ± 0.11 |
| Liver | 1.89 ± 0.32 | 1.32 ± 0.46 | 1.32 ± 0.23 | 0.88 ± 0.13 |
| Spleen | 1.87 ± 0.43 | 1.60 ± 0.46 | 1.28 ± 0.18 | 0.85 ± 0.13 |
| Kidney | 2.80 ± 0.44 | 0.94 ± 0.25 | 0.89 ± 0.11 | 0.68 ± 0.06 |
| Muscle | 0.10 ± 0.02 | 0.15 ± 0.05 | 0.19 ± 0.01 | 0.17 ± 0.01 |
| Fat | 0.10 ± 0.03 | 0.15 ± 0.06 | 0.23 ± 0.03 | 0.22 ± 0.03 |
| Heart | 0.84 ± 0.05 | 0.32 ± 0.08 | 0.36 ± 0.03 | 0.32 ± 0.01 |
| Brain | 0.98 ± 0.11 | 0.34 ± 0.1 | 0.30 ± 0.03 | 0.26 ± 0.01 |
| Bone | 0.44 ± 0.06 | 0.30 ± 0.08 | 0.39 ± 0.04 | 0.44 ± 0.06 |
| Pancreas | 1.39 ± 0.25 | 0.63 ± 0.19 | 0.51 ± 0.03 | 0.35 ± 0.01 |

TABLE 7
The effect of known σ1R compound on biodistribution of (−)[18F]TZ9580
at 60 min post injection in male Sprague Dawley rats (mean ± SD, % ID/g, n = 3~4).

| | | (−)[₁₈F]TZ9580 + | (−)[₁₈F]TZ9580 + |
|---|---|---|---|
| Organs | (−)[₁₈F]TZ9580 | (+)Pentazocine | (−)TZ3108 |
| Blood | 0.36 ± 0.05 | 0.45 ± 0.04 | 0.38 ± 0.11 |
| Lung | 1.84 ± 0.26 | 1.52 ± 0.08**** | 1.59 ± 0.28* |
| Liver | 1.65 ± 0.16 | 1.56 ± 0.04 | 0.95 ± 0.24**** |
| Spleen | 1.16 ± 0.06 | 1.04 ± 0.1 | 0.93 ± 0.19* |
| Kidney | 1.10 ± 0.06 | 0.89 ± 0.09** | 0.86 ± 0.12* |
| Muscle | 0.21 ± 0.02 | 0.25 ± 0.01 | 0.22 ± 0.06 |
| Fat | 0.28 ± 0.05 | 0.27 ± 0.01 | 0.26 ± 0.09 |
| Heart | 0.37 ± 0.03 | 0.42 ± 0.01 | 0.40 ± 0.07 |
| Brain | 0.34 ± 0.02 | 0.35 ± 0.02 | 0.32 ± 0.05 |
| Pancreas | 0.58 ± 0.06 | 0.44 ± 0.04 | 0.42 ± 0.06 |

### In Vitro Autoradiography Analysis of (−)[18F]TZ9580 in Rat Brain:

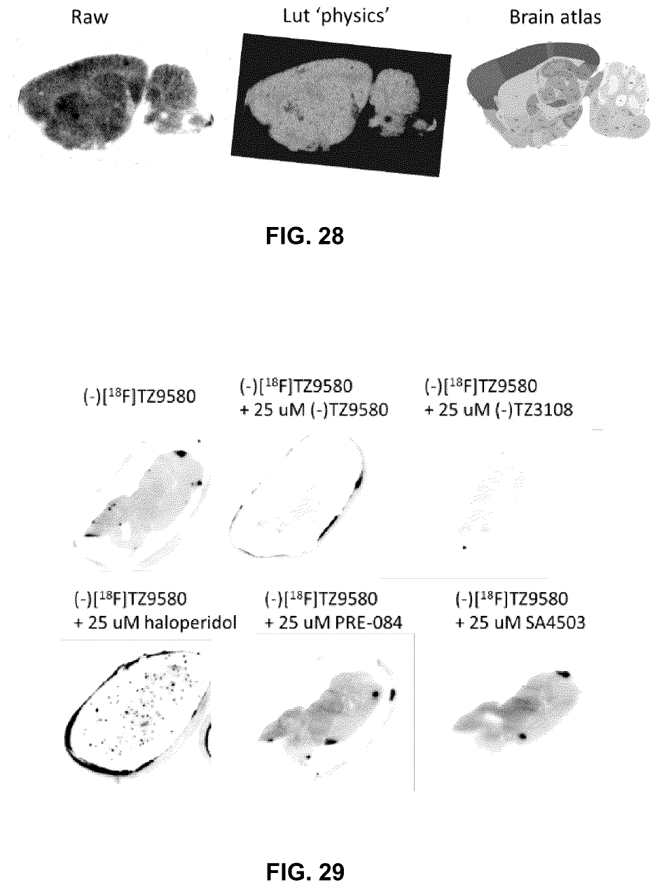

In order to examine the distribution of (−)[18F]TZ9580 in the CNS, we performed an autoradiography analysis in the adult SD rat brain. In general, (−)[18F]TZ9580 showed high uptakes in the gray matter-rich regions of the brain including the cortex, striatum, hippocampus, thalamus, and gray matter of cerebellum; whereas a low tracer uptake was identified in the white matter region such as corpus callosum and white matter of cerebellum. The distribution of (−)[18F]TZ9580 matched well with immunohistochemistry staining using anti-σ1R antibody, indicating the specificity of (−)[18F]TZ9580 (FIG. 28).

To further confirm the specificity of (−)[18F]TZ9580, we next performed an autoradiography analysis using (−)[18F]TZ9580 with blocking of selected known σ1R compounds. In the presence of 25 μM of selected blocking agents, self-block using (−)TZ9580 showed almost a completed block in the uptake of (−)[18F]TZ9580 (FIG. 29). Similarly, blocking using the analogs of TZ9580, TZ3-108, also showed a dramatic decrease in the uptake of (−)[18F]TZ9580. In addition, haloperidol, a widely used σ1R antagonist, also showed almost a complete blocking of (−)[18F]TZ9580 uptake in the rat brain. In contrast, treatment with known σ1R agonists, PRE-084 and SA4503, showed no significant effect of blocking but a minor increase of (−)[18F]TZ9580 uptake (FIG. 29).

### In Vivo PET Analysis of (−)[18F]TZ9580 in Mouse Brain:

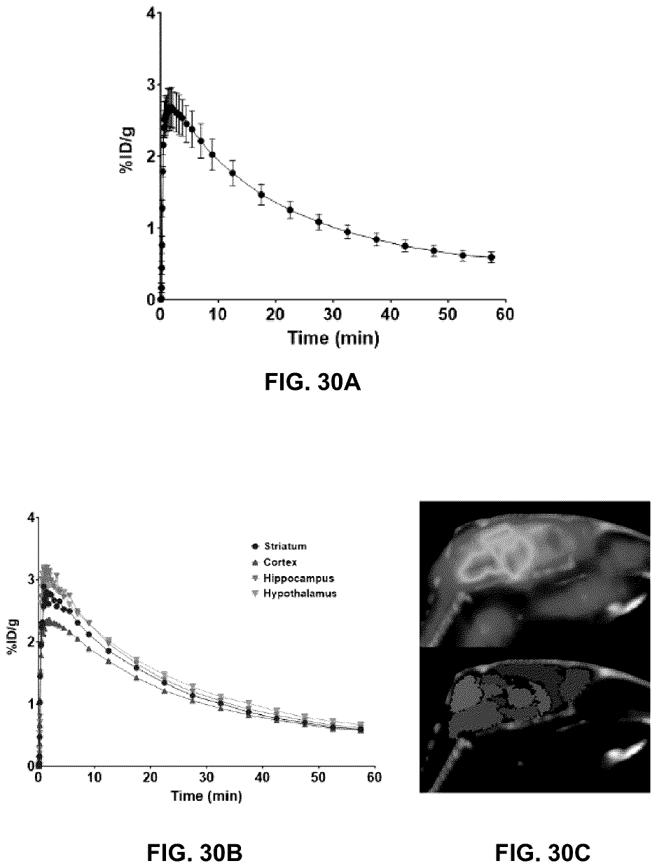

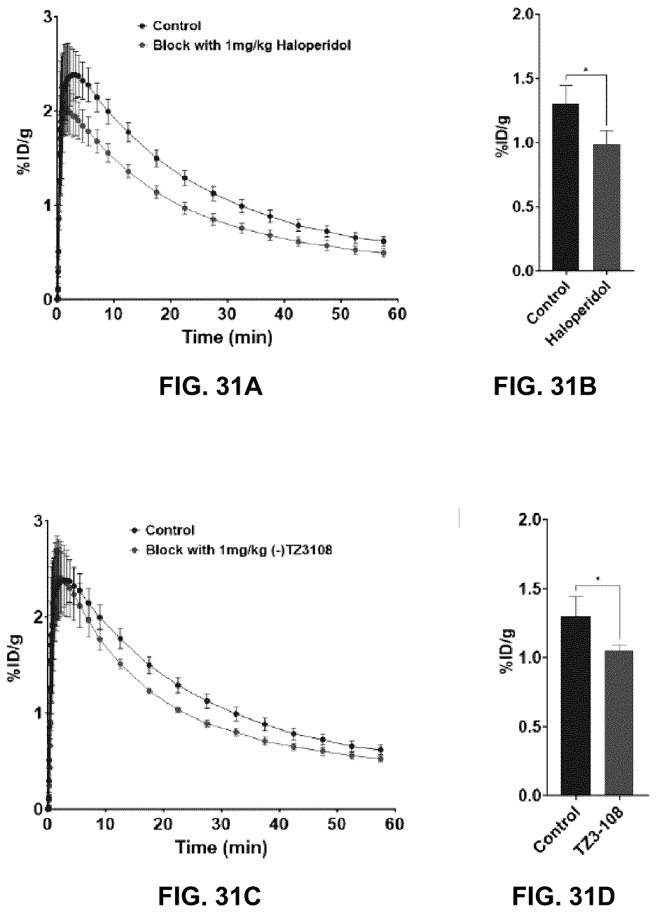

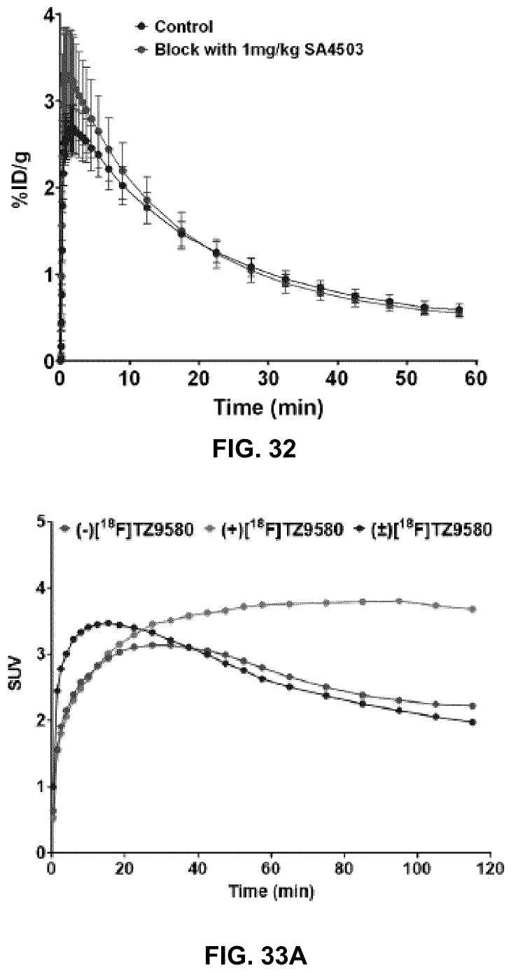

We next performed a PET imaging analysis of (−)[18F]TZ9580 in 12 weeks old CD1 mouse brain. In general, (−)[18F]TZ9580 showed a high brain uptake in mouse brains. It entered the mouse brain fast and the brain uptake reached the peak with a % ID/g value of 2.68 at 2 min and then gradually washed out from the brain with an uptake of 2.02, 1.47, 0.94, and 0.68 at 10, 20, 30, and 40 min respectively (FIG. 30A). The uptake was high in the majority of different subregions of the brain such as striatum, cortex, hippocampus, and hypothalamus (FIG. 30B, FIG. 30C). To test the in vivo binding specificity, we next performed a blocking study using well well-known σ1R ligand, haloperidol, and our previously published σ1R ligand, (−)TZ3108. Mice were pretreated with saline, 1 mg/kg of haloperidol, or 1 mg/kg of (−)TZ3108 5 min prior to the administration of (−)[18F]TZ9580. As expected, both haloperidol and (−)TZ3108 showed a significant impact on the uptake of (−)[18F]TZ9580 in mouse brains (FIG. 31A, FIG. 31C). The average uptake of (−)[18F]TZ9580 from 20 to 30 min in the mouse brain was significantly reduced by 24.4% and 19.3% by pretreatment with haloperidol and (−)TZ3108 respectively with P values of 0.036 and 0.040 in t-test, indicating the binding of (−)[18F]TZ9580 towards σ1R is specific in vivo (FIG. 31B, FIG. 31D). Interestingly, blocking with SA4503, another known σ1R agonist, did not block the uptake of (−)[18F]TZ9580, and instead, increased the uptake of (−)[18F]TZ9580 in the brain (FIG. 32).

### In Vivo PET Analysis of (−)[18F]TZ9580 in Non-Human Primate Brain:

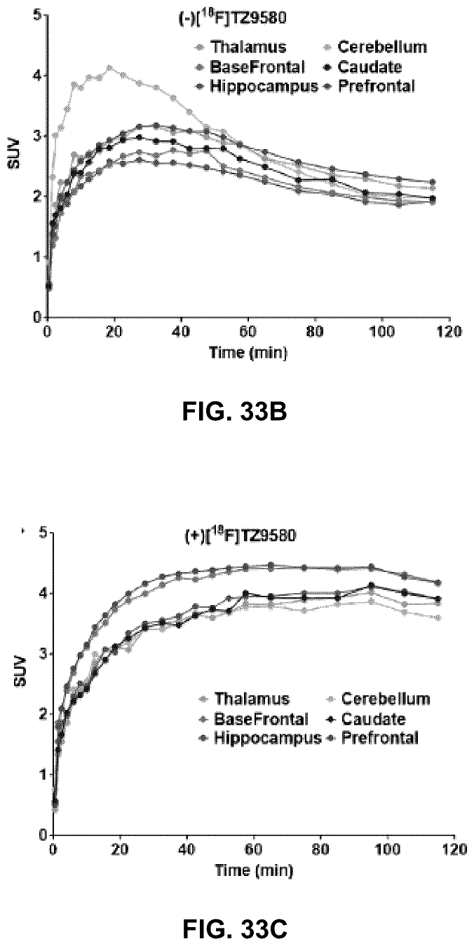

To evaluate the dynamics and the distribution of (−)[18F]TZ9580 in different brain regions, PET analysis of (−)[18F]TZ9580 was performed in NHP brain. In general, (−)[18F]TZ9580 entered the brain very well with a peak SUV of 3.47 at 15 min post injection of tracer. The tracer penetrated the blood brain barrier and entered the brain quickly, reached the peak uptake and then gradually washed out from the brain with SUVs of 3.40, 2.99, 2.37, and 1.97 at 20, 40, 80, 120 min respectively. Within the brain, (−)[18F]TZ9580 showed a the highest uptake in the cerebellum with a peak SUV at 4.20 at 20 min, the uptake in other regions such as cortex, hippocampus, and striatum were also high with a peak SUV from 2.5 to 3.2 (FIG. 33A, FIG. 33B). In comparison, (+)[18F]TZ9580 showed a similar high uptake in the brain but a slightly different dynamics. (+)[18F]TZ9580 gradually entered the brain with SUVs of 3.15, 3.58, 3.75, and 3.79 at 20, 40, 60, and 80 min respectively, and reached to the peak SUV of 3.80 at 100 min, and then slowly washed out with a SUV of 3.68 at 120 min. Within the brain, the uptake was high in all tested regions with cortex showed a higher uptake (FIG. 33A, FIG. 33C). Additionally, we also compared the uptake of racemic mixture (±)[18F]TZ9580, whereas (±)[18F]TZ9580 showed a similar dynamics to (−)[18F]TZ9580 with a bit higher uptake similar to (+)[18F]TZ9580.

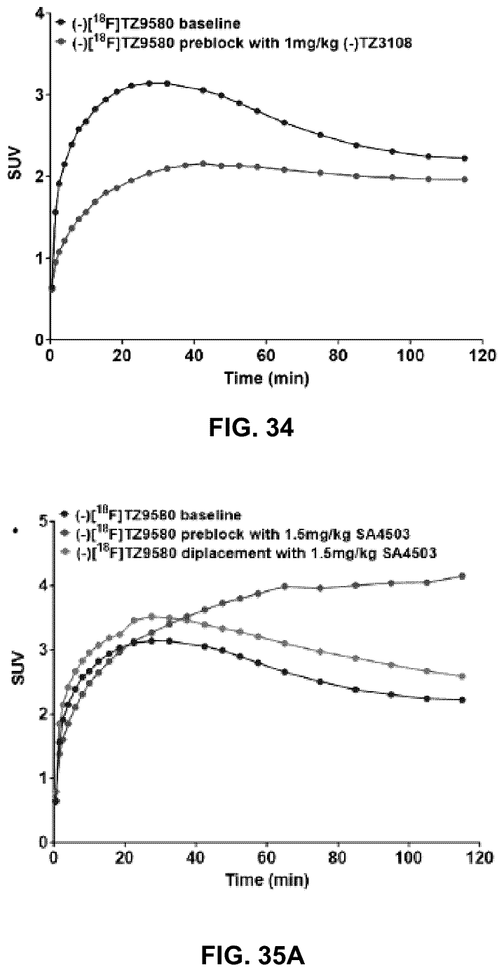

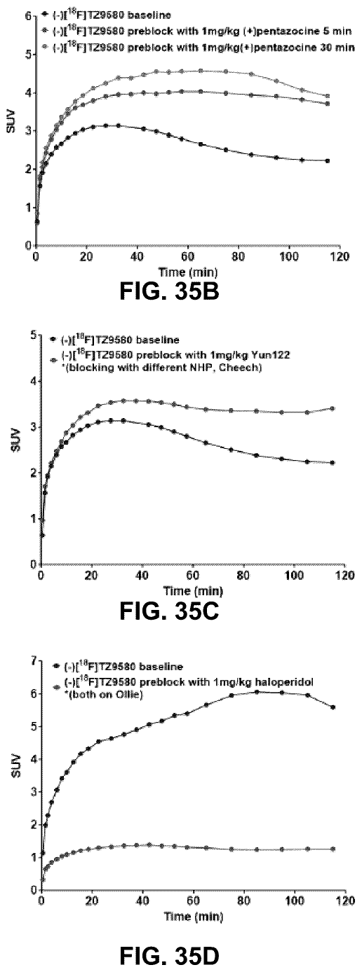

We further tested the in vivo specificity of (−)[18F]TZ9580 in the brain by preblocking with our previously published σ1R compound (−)TZ3108. Preblocking with 1 mg/kg (−)TZ3108 5 min prior to the administration of (−)[18F]TZ9580 significantly reduced the tracer uptake by 30% (average uptake from 20 to 60 min), with a peak SUV at 2.16 in comparison with 3.47 in baseline scan (FIG. 34). In addition, we also tested the uptake of (−)[18F]TZ9580 with preblock or displacement of other known σ1R compounds, including SA4503, (+)pentazocine, Yun122, and haloperidol. Surprisingly, all compounds showed an induction instead of blocking, possibly due to the occupation of σ1R by blocking agents in peripheral tissues resulted in an enhanced influx of the tracer to the brain (FIG. 35A, FIG. 35B, FIG. 35C, FIG. 35D). However, these changes still confirm the interaction and the specificity between our novel compound TZ9580 to σ1R in vivo.

### Radiometabolite Analysis of (−)[18F]TZ9580 in Non-Human Primate Plasma Sample:

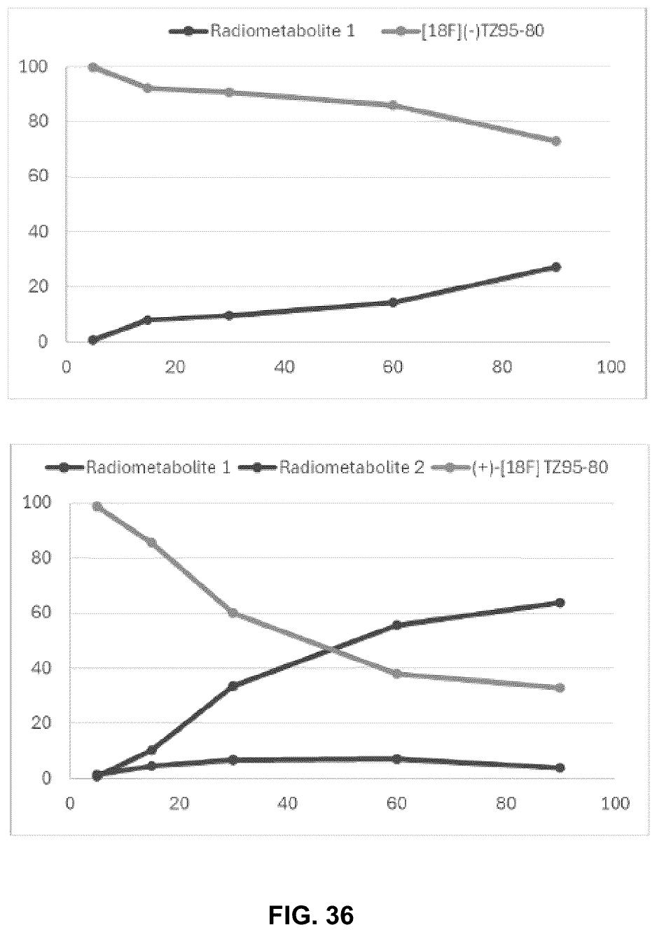

To test the in vivo stability of (−)[18F]TZ9580, we also tested the radiometabolite in NHP plasma samples. Plasma radiometabolite analysis was also performed for macaque plasma samples collected at 5, 15, 30, 60, and 90 min post-injection of each radiotracer. HPLC analysis of NHP plasma showed (−)[18F]TZ9580 was metabolized slowly in vivo with a percentage of 99.51%, 90.53%, and 72.78% remained parent compounds at 5, 30, and 90 min post injection respectively (FIG. 36). A single peak of radiometabolite was identified with a percentage of 0.49, 9.47, and 27.22% of parent compounds at 5, 30, and 90 min post injection respectively indicating (−)[18F]TZ9580 is relatively stable in vivo with a slow radiometabolite rate. In contrast, (+)[18F]TZ9580 showed a relatively fast radiometabolite rate with percentage of 98.51%, 59.97%, and 32.61% remained parent compounds at 5, 30, and 90 min post injection (FIG. 36). Additionally, two peaks of radiometabolites were identified with a percentage of 1.08%, 6.68%, and 3.76% of parent compound for radiometabolite 1 and 0.42%, 33.25%, and 63.63% of parent compound for radiometabolite 2 respectively, indicating a (+)[18F]TZ9580 is less stable than (−)[18F]TZ9580 in vivo.

### Radiometabolite Analysis of (−)[18F]TZ9580 in Rat Plasma and Brain Sample:

To test if the identified radiometabolite can penetrate blood brain barrier and confound the PET measurement, we also carried out the radiometabolite in the rat brain sample (Table 8). At 60 min post injection of (−)[18F]TZ9580, two radiometabolites were identified in the rat plasma with a percentage of 71% remained as parent compound and 12% of radiometabolite 1 and 17% of radiometabolite 2, different from one peak of radiometabolite 1 in NHP plasma. Additionally, in the brain, no radiometabolite 1 was identified but only radiometabolite 2 was identified. The percentage of parent compound was 22% and radiometabolite 2 was 78% 60 min post injection. While the radiometabolite 2 was detected in the brain, radiometabolite 1 was not, indicating PET imaging of NHP brain cannot be confounded by the radiometabolite.

TABLE 8
Radiometabolite analysis of (−)[18F]TZ9580
in rat plasma and rat brain homogenates.

| Time Point (min) | Peak 1 (%) | Peak 2 (%) | Parental peak (%) |
|---|---|---|---|
| 60 min brain | 0 | 22 | 78 |
| 60 min plasma | 12 | 71 | 17 |

### In Vivo PET Analysis of (−)[18F]TZ9580 in Alzheimer Mouse Models:

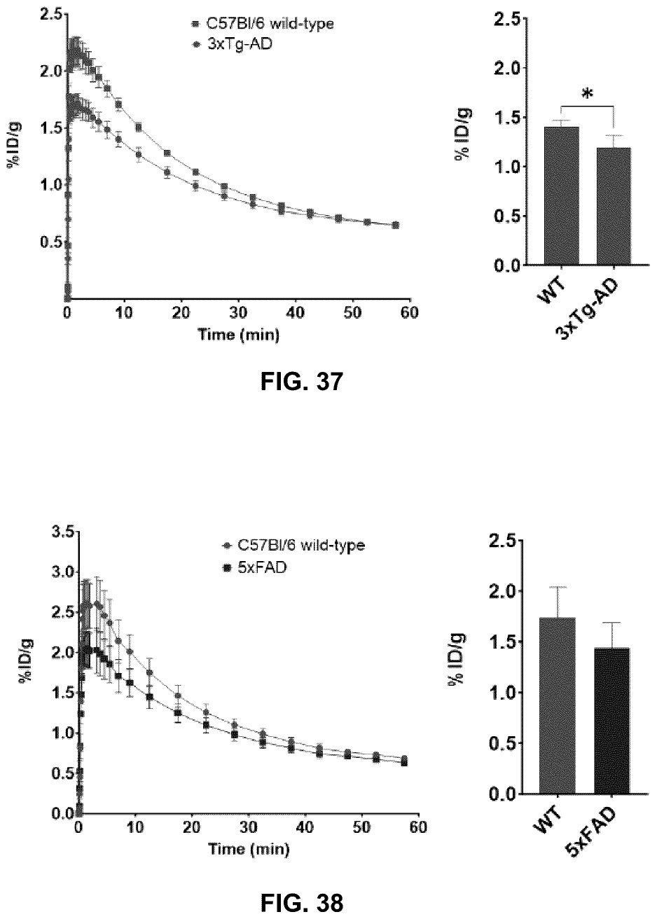

We next test the uptake of our novel σ1R specific radioligand (−)[18F]TZ9580 in the Alzheimer mouse model brains. We first performed microPET imaging of (−)[18F]TZ9580 in the brain of 9-month-old 3xTg-AD transgenic mouse model of Alzheimer disease and age-matched C57/Bl6 wild type mice (FIG. 9). In general, the uptake of (−)[18F]TZ9580 in C57/Bl6 mouse brain was high and reached the peak at % ID/g of 2.16 at 1.5 min post injection and gradually washed out from brain with the uptake of 1.50, 1.11, and 0.81 at 10, 20, and 40 min post injection. Interestingly, the uptake of (−)[18F]TZ9580 in the age-matched 3xTg-AD mouse brain was significantly lower than wild-type mice that two-way ANOVA showed F(1, 8)=11.67 with a P value of 0.0091 (FIG. 37). The average uptake of (−)[18F]TZ9580 from 10 to 20 min of the scan was 15% lower in 3xTg-AD mouse brain compared to wild-type with a P value of 0.01 using t-test (FIG. 37). In addition to 3xTg-AD mice, we also tested the uptake of (−)[18F]TZ9580 in 16 month old 5xFAD mice and age-matched C57/Bl6 wild type mice (FIG. 38). Similarly, the uptake of brain (−)[18F]TZ9580 was higher in wild-type mice and lower in 5xFAD mice that two-way ANOVA showed F(1, 4)=2.51 with a P value of 0.1882. The average uptake of (−)[18F]TZ9580 from 10 to 20 min from 5xFAD mice was 23.6% lower than wild-type mice though t-test showed a P value of 0.14 (FIG. 38).

### Ex Vivo Biodistribution of (−)[11C]TZ3114 in SD Rats:

The ex vivo biodistribution analysis of (−)[11C]TZ3114 was performed in male Sprague Dawley (SD) rats to evaluate the tracer dynamic and uptake of (−)[11C]TZ3114 in different organs. The uptake of (−)[11C]TZ3114 was high in the lung, liver, spleen, kidney, heart, pancreas, and brain with a % ID/g value of 17.83, 2.01, 3.18, 6.20, 1.89, 1.86, and 1.87 at 5 min post injection respectively. (−)[11C]TZ3114 entered these organs quickly and then washed out from these organs. For example, the uptake of (−)[11C]TZ3114 in the lung was 17.83, 4.90, and 2.33 at 5 min, 30 min, and 60 min post injection of tracer whereas the uptake of (−)[11C]TZ3114 in the brain was 1.87, 0.88, and 0.40 at 5, 30, and 60 min post injection of tracer (Table 9). To test the specificity of (−)[11C]TZ3114, we also examined the biodistribution of (−)[11C]TZ3114 with preblocking with known σ1R compound, FTC146, at 1 mg/kg and administrated 5 min before the injection of tracer. The organ of interested was collected at 30 min post injection. Surprisingly, the uptake of (−)[11C]TZ3114 with preblocking of FTC146 was similar with no treatment control. Additionally, preblocking with 2 mg/kg of haloperidol also showed no significant impact on the uptake of (−)[11C]TZ3114 in lung, liver, and subregions of the brain (Table 10).

TABLE 9
Biodistribution of (−)[11C]TZ3114 in male Sprague Dawley
rats (mean ± SD, % ID/g, n = 4).

| | | | 30 min + 1 mg/ | |
|---|---|---|---|---|
| | 5 min | 30 min | kg FTC146 | 60 min |
| Blood | 0.08 ± 0.01 | 0.06 ± 0.01 | 0.06 ± 0.01 | 0.06 ± 0.01 |
| Lung | 17.83 ± 3 | 4.9 ± 1.59 | 5.15 ± 0.21 | 2.33 ± 0.38 |
| Liver | 2.01 ± 0.76 | 2.97 ± 0.51 | 2.98 ± 0.44 | 2.7 ± 0.37 |
| Spleen | 3.18 ± 1.46 | 2.88 ± 0.31 | 3.46 ± 0.61 | 2.77 ± 0.4 |
| Kidney | 6.2 ± 1.75 | 2.93 ± 0.44 | 3.07 ± 0.27 | 1.7 ± 0.26 |
| Muscle | 0.12 ± 0.06 | 0.15 ± 0.02 | 0.16 ± 0.02 | 0.12 ± 0.01 |
| Fat | 0.1 ± 0.03 | 0.18 ± 0.06 | 0.16 ± 0.03 | 0.19 ± 0.04 |
| Heart | 1.89 ± 0.4 | 0.41 ± 0.04 | 0.49 ± 0.03 | 0.3 ± 0.02 |
| Brain | 1.87 ± 0.26 | 0.88 ± 0.18 | 0.88 ± 0.08 | 0.4 ± 0.1 |
| Pancreas | 1.86 ± 0.55 | 1.26 ± 0.22 | 1.19 ± 0.12 | 0.9 ± 0.13 |

TABLE 10
Biodistribution of (−)[11C]TZ3114 in male Sprague Dawley
rats (mean ± SD, % ID/g, n = 4).

| | | 30 min w/ | 30 min w/ |
|---|---|---|---|
| | 30 min | haloperidol | (−)TZ3108 |
| Blood | 0.05 ± 0.01 | 0.05 ± 0.01 | 0.04 ± 0.01 |
| Lung | 3.78 ± 1.06 | 4.24 ± 0.5 | 5.06 ± 1.32 |
| Liver | 1.81 ± 0.31 | 2.03 ± 0.31 | 1.9 ± 0.21 |
| Muscle | 0.1 ± 0.03 | 0.15 ± 0.03 | 0.11 ± 0.02 |
| Fat | 0.13 ± 0.02 | 0.19 ± 0.05 | 0.11 ± 0.04 |
| Cerebellum | 0.6 ± 0.15 | 0.52 ± 0.08 | 0.68 ± 0.24 |
| Cortex | 0.79 ± 0.1 | 0.82 ± 0.1 | 0.92 ± 0.09 |
| Striatum | 0.67 ± 0.12 | 0.64 ± 0.1 | 0.72 ± 0.12 |
| Hippocampus | 0.76 ± 0.12 | 0.73 ± 0.12 | 0.86 ± 0.21 |

### In Vivo PET Analysis of (−)[11C]TZ3114 in Mouse Brain:

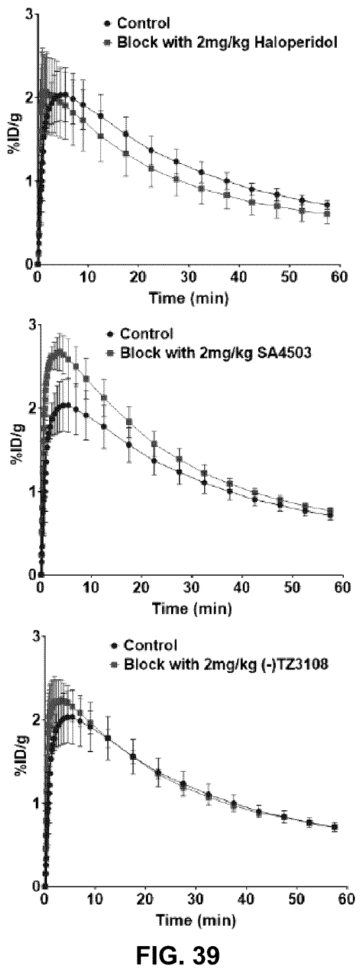

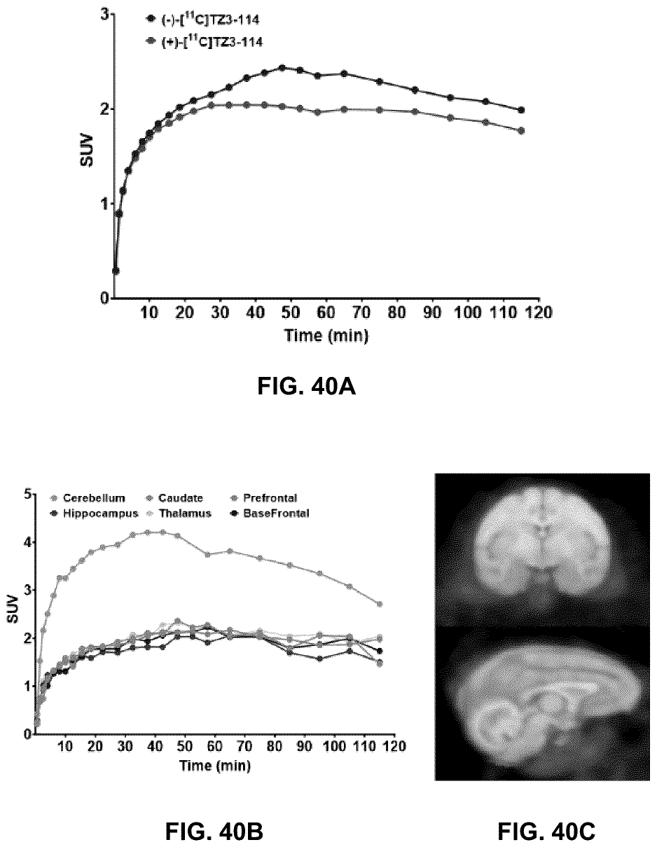

We next performed a PET imaging analysis of (−)[11C]TZ3114 in 12 week old CD1 mouse brain. In general, (−)[11C]TZ3114 showed a high brain uptake with a peak % ID/g of 2.04 at 5 min and then gradually washed out from the brain with uptakes of 1.24 and 0.74 at 27 and 60 min respectively. Preblocking with 2 mg/kg σ1R-specific ligand haloperidol showed a reduced uptake of (−)[11C]TZ3114 indicating (−)[11C]TZ3114 is specifically bound to σ1R in vivo. Interestingly, pretreatment with well well-known σ1R agonist, SA4503, induced the uptake of (−)[11C]TZ3114. No significant impact was identified in the uptake of (−)[11C]TZ3114 with pretreatment of (−)TZ3114 (FIG. 39).

### In Vivo PET Analysis of (−)[11C]TZ3114 in Non-Human Primate Brain:

To examine the tracer dynamics and the distribution in different brain regions, we performed PET analysis of (−)[11C]TZ3114 in NHP brain. In general, (−)[11C]TZ3114 entered the NHP well with a peak uptake SUV value of ˜2.43 at 50 min. The tracer gradually entered the brain and then slowly washed out from the brain (FIG. 30A, FIG. 30C). Among different regions of the brain, the uptake (−)[11C]TZ3114 was very high in the cerebellum with a peak SUV of ˜4.20 at 40 min. The uptake in the striatum, hippocampus, and cortex were similar with a peak SUV of ˜2.1 (FIG. 30B, FIG. 30C). Additionally, we also radiolabeled (+)[11C]TZ3114 and compared its uptake with (−)[11C]TZ3114 in the same NHP brain. The uptake of (+)[11C]TZ3114 was slightly lower than (−)[11C]TZ3114 with a peak uptake at 2.04 at 27 min.

### Radiometabolite Analysis of (−)[11C]TZ3114 in Non-Human Primate Plasma Sample:

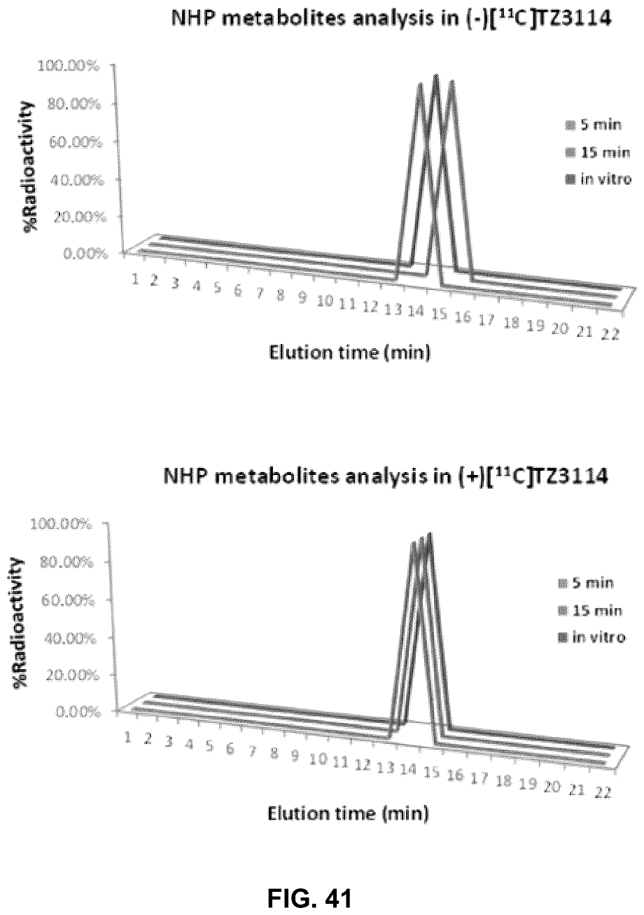

To test the in vivo stability of (−)[11C]TZ3114, we also tested the radiometabolite in NHP plasma samples. Plasma radiometabolite analysis was also performed for macaque plasma samples collected at 5-, and 15-min post-injection of each radiotracer. HPLC analysis of NHP plasma for (−)[11C]TZ3114 at 5, and 15 min revealed only a single radioactive peak, which is the parent compound (−)[11C]TZ3114. At 5- and 15-minutes post-injection, the solvent extract of plasma contained 100% parent compound. Similarly, radiometabolites of NHP plasma for (+)[11C]TZ3114 at 5, and 15 minutes revealed only a single radioactive peak, which is the parent compound (+)[11C]TZ3114 (elution time 14-15 min). At all-time points of post-injection, the solvent extract of plasma contained 100% parent compound (FIG. 41). The metabolite results showed no significant lipophilic radiometabolite was observed for both (−)[11C]TZ3114 and (+)[11C]TZ3114 at 15 min post-injection, and both isomers had similar metabolite stability in nonhuman primate plasma post injection.

### In Vivo PET Analysis of (−)[11C]TZ9667 in Non-Human Primate Brain:

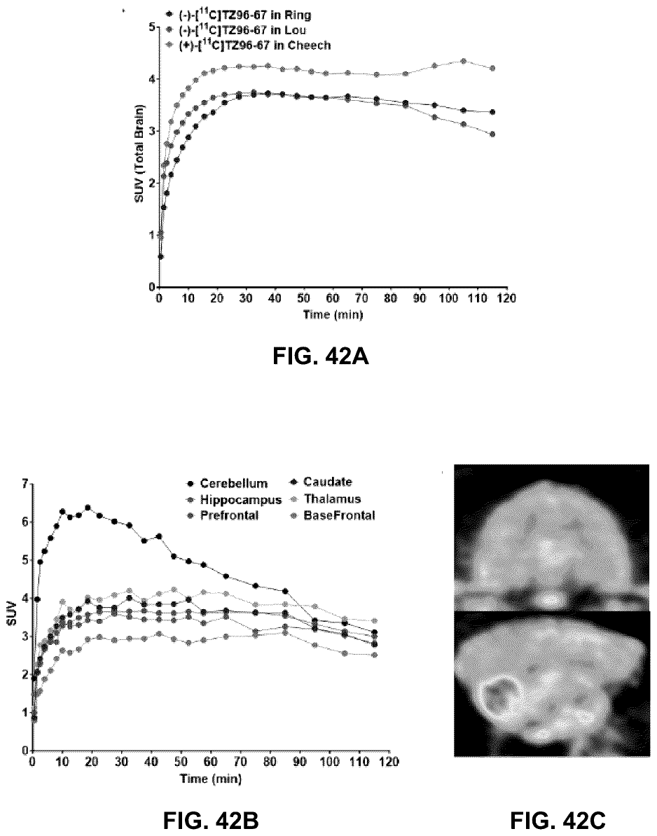

To examine the tracer dynamics and the distribution in different brain regions, we performed PET analysis of (−)[11C]TZ9667 in NHP brain. In general, (−)[11C]TZ9667 entered the NHP well with a peak uptake SUV value of ˜3.7 at around 20 to 30 min. The tracer entered the brain and then slowly washed out from the brain (FIG. 42A, FIG. 42C). Among different regions of the brain, the uptake (−)[11C]TZ9667 was very high in the cerebellum with a peak SUV of -6.3 at 20 min. The uptake in the striatum, hippocampus, and cortex were similar with a peak SUV of ˜4.0, ˜3.6, and ˜3.6 respectively (FIG. 42B, FIG. 42C). We also examined the test-retest reliability of (−)[11C]TZ9667 in two different monkeys, the uptake of (−)[11C]TZ9667 between two NHPs were almost identical (FIG. 42A) indicating the uptake (−)[11C]TZ9667 is consistent in different NHPs. Additionally, we also radiolabeled (+)[11C]TZ9667 and compared its uptake with (−)[11C]TZ9667. The uptake of (+)[11C]TZ9667 was slightly higher than (−)[11C]TZ9667 with a peak uptake at ˜4.2 at 40 min (FIG. 42A).

### In Vivo PET Analysis of [18F]TZ3108 in Non-Human Primate Brain:

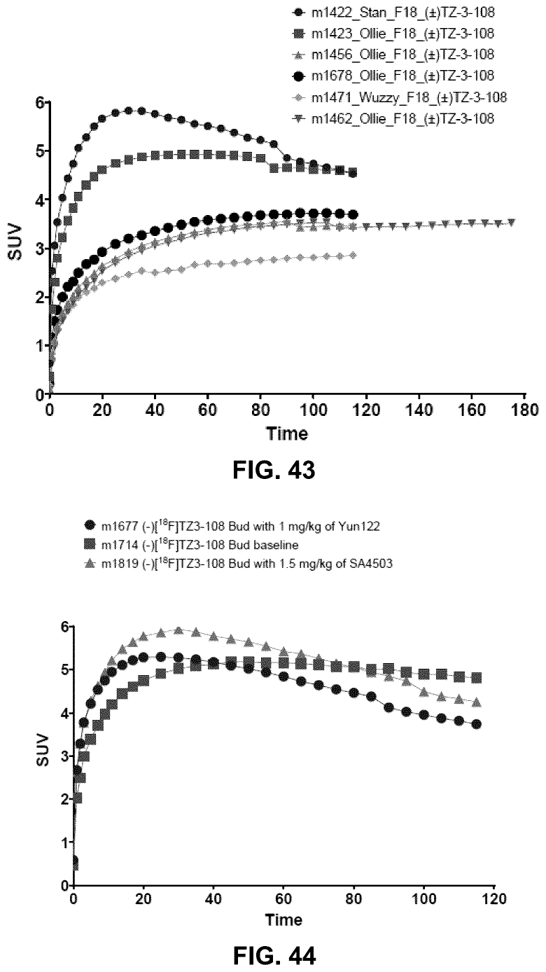

To evaluate the dynamics and distribution of [18F]TZ3108 in the brain, we performed PET analysis of (±)[18F]TZ3108, (+)[18F]TZ3108, and (−)[18F]TZ3108 in the NHP brain. (±)[18F]TZ3108 data was obtained from 2011 to 2014 and was re-analyzed using current image analysis protocol. In general, the uptake was high in all NHPs. Stan showed the highest uptake with a much higher SUV than other individuals and also showed a fast washed out. Ollie showed a consistent SUV for most of the scans. Wuzzy showed a bit lower SUV than other NHPs (FIG. 43).

Blocking studies were performed on (−)[18F]TZ3108 from 2014 to 2015 on Bud. Yun122 at 1 mg/kg and SA4503 at 1.5 mg/kg was administered 5 min before the dose. The overall uptake of (−)[18F]TZ3108 was similar between baseline and blocking, both Yun122 and SA4503 affected the tracer dynamics significantly was a bit higher uptake at beginning of the scan with peak SUVs of 5.28 at 20 min for Yun122 and 5.94 at 30 min for SA4503, and then a much faster brain washed out rate and thus a lower uptake at later of the scan with SUVs of 4.26 and 3.74 at 120 min respectively. In contrast, the baseline scan showed a much slow washed-out rate with a peak SUV of 5.19 at 45 min of the scan and 4.82 at 120 min (FIG. 44).

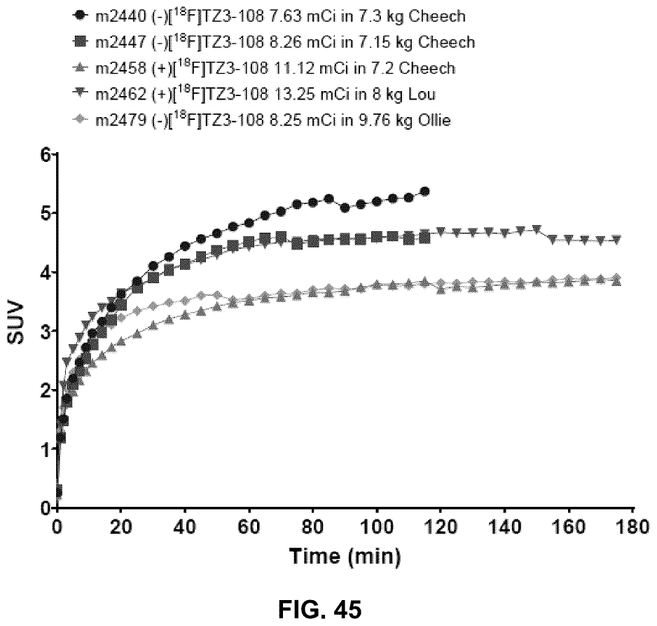

Laster studies in older NHPs showed a slightly different tracer dynamics of both (−)[18F]TZ3108 and (+)[18F]TZ3108. Both isomers entered the brain quickly and then showed a relatively stable dynamics with no significant brain washed out identified during 120 or 180 min of the scan (FIG. 45).

### In Vivo PET Analysis of [18F]TZ3108 in Alzheimer Mouse Brain:

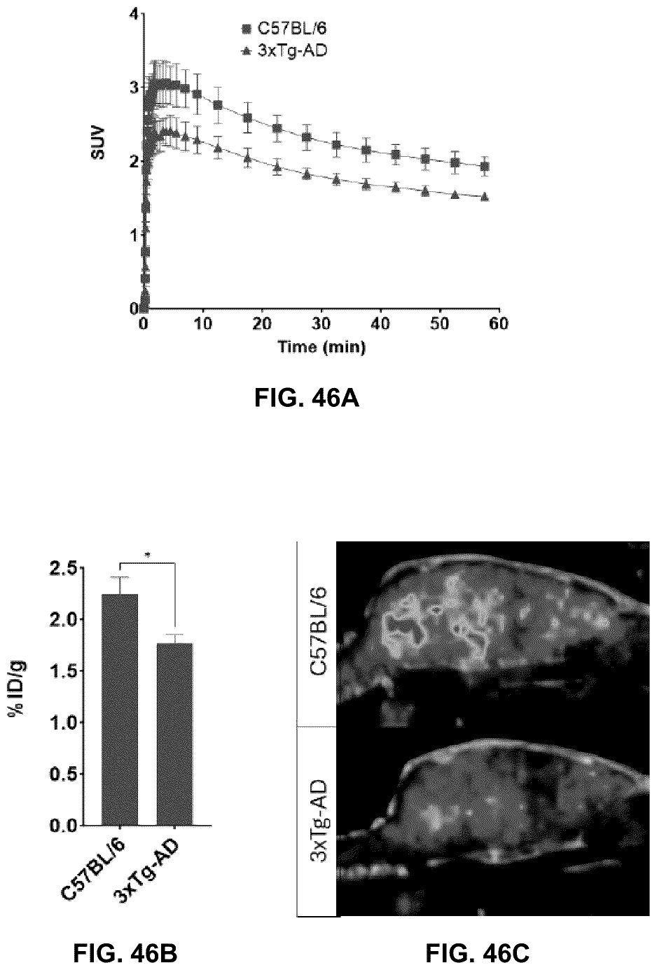

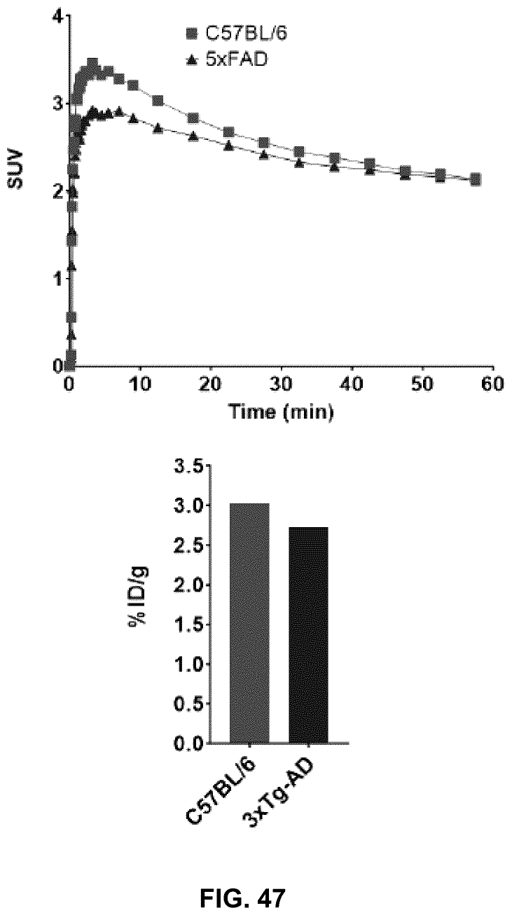

We next test the uptake of our novel σ1R specific radioligand (−)[18F]TZ3108 in the Alzheimer mouse model brains. We first performed microPET imaging of (−)[18F]TZ3108 in the brain of 10 month old 3xTg-AD transgenic mouse model of Alzheimer disease and age-matched C57/Bl6 wild type mice (FIG. 46A). In general, the uptake of (−)[18F]TZ3108 in C57/Bl6 mouse brain was high and reached the peak at % ID/g of 3.08 at 4 min post injection and gradually washed out from brain with the uptake of 3.02, 2.38, and 2.07 at 10, 30, and 60 min post injection. Interestingly, the uptake of (−)[18F]TZ3108 in the age-matched 3xTg-AD mouse brain was significantly lower than wild-type mice that two-way ANOVA showed F(1, 4)=19.62 with a P value of 0.0114 (FIG. 46A). The average uptake of (−)[18F]TZ3108 from 20 to 40 min of the scan was 21.2% lower in 3xTg-AD mouse brain compared to wild-type with a P value of 0.01 using t-test (FIG. 46B). In addition to 3xTg-AD mice, we also tested the uptake of (−)[18F]TZ3108 in 17 month old 5xFAD mice and age-matched C57/Bl6 wild type mice. The average uptake of (−)[18F]TZ3108 from 10 to 20 min from 5xFAD mice was 10% lower than wild-type mice (FIG. 47).

### Example 5—Radiosynthesis and In Vivo Evaluation of Six F-18 Radioligands for Imaging Sigma-1 Receptor in the Brain

Objectives: The Sigma-1 receptor (σ1R) is a key biomarker in neurodegenerative diseases. Significant efforts have focused on developing clinically suitable F-18 labeled PET radiotracers to measure changes in σ1R expression associated with various conditions, including Alzheimer's and other neuropsychiatric disorders. Motivated by the promising outcomes of treating Alzheimer's disease with σ1R inhibitors, along with our advancements in development of PET σ1R radiotracers, our goal is to develop a clinic suitable F-18 labeled PET radiotracer for imaging σ1R in the central nervous system (CNS). Here, we present our efforts on the design, synthesis and validation of five novel F-18 labeled radiotracers for imaging σ1R in the brain, including one previously reported by our group.

Methods: We synthesized and radiosynthesized five new F-18 radiotracers: (±)-[18F]1 along with its enantiopure isomers (−)-[18F]1 and (+)-[18F]1, as well as (−)-[18F]2, and, (−)-[18F]3 under optimized conditions. we determined in vitro binding potency for the σ1R and selectivity for over σ2R and VAChT using radioactive competitive assay. Two F-18 radiochemistry strategies were used to radiosynthesize of these new five F-18 radiotracers. The (±)-[18F1, (−)-[18F]1, (+)-[18F]1, and (−)-[18F]2 were radiolabeled with K[18F]/F— using a two-step procedure: 1) nucleophilic substitution of the ditosylate precursor with dried K[18F]/F—; 2) O-alkylation of the phenol precursor with 2-[18F]fluoroethyl tosylate in the presence of cesium carbonate, while (−)-[18F]3 was radiosynthesized using an innovative ruthenium-mediated radiofluorination ([18F]/F—) chemistry on an aromatic phenol precursor. PET imaging brain studies of each radiotracer were first performed using Focus 220 PET scanner on male cynomolgus macaques. Dynamic data were acquired from 0.0-120 minutes, and tissue time-activity curves in the brain were obtained. Secondly, PET studies of radiotracer(s) were performed in normal CD mice and CD mice pretreated using σ1R ligand, (±)-TZ3108, haloperidol. Thirdly, PET brain imaging study of (−)-[18F]1 were performed for 3xTg-AD mouse model of Alzheimer and age-matched wild-type using a Siemens Inveon MM PET/CT scanner. Biodistribution study of (−)-[18F]1 was performed using Sprague-Dawley rats, euthanized at 5, 30, 60, and 120 minutes post injection. Radiometabolism analysis of (−)-[18F]1 was conducted for macaque arterial plasma samples collected at 5, 15, 30, 60, and 90 minutes during PET scan of (−)-[18F]1.

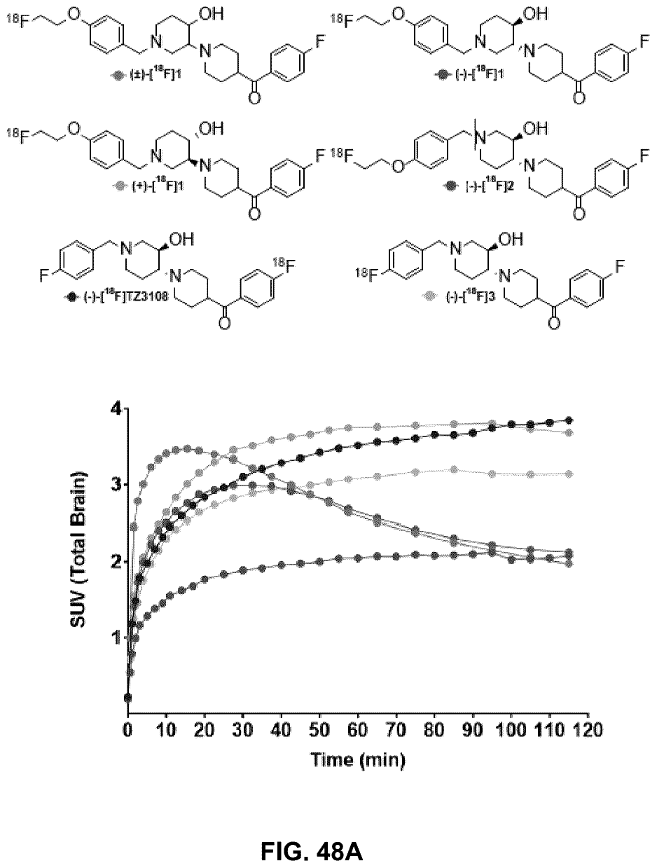

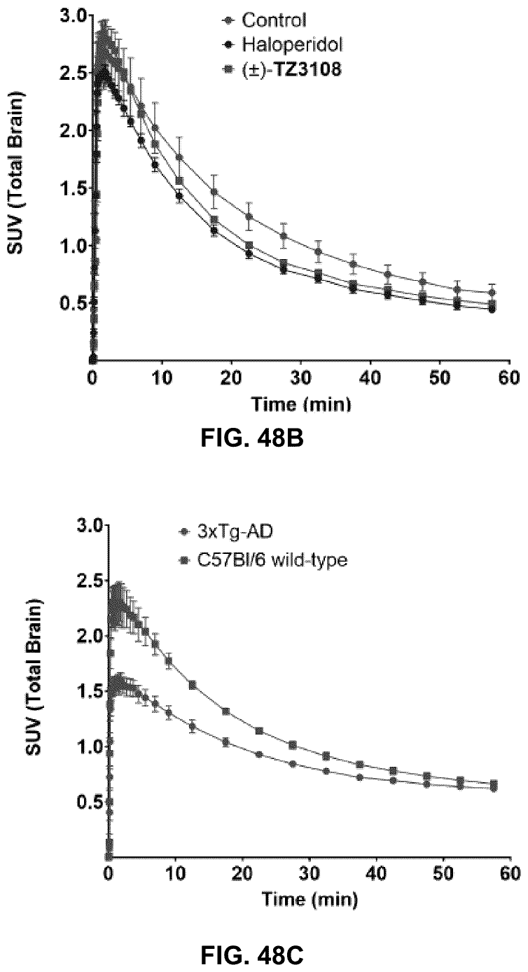

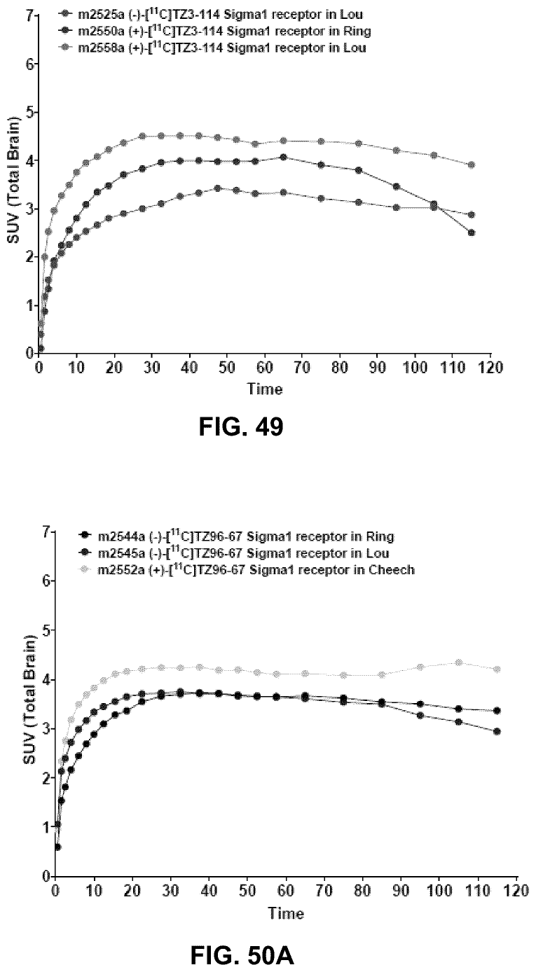

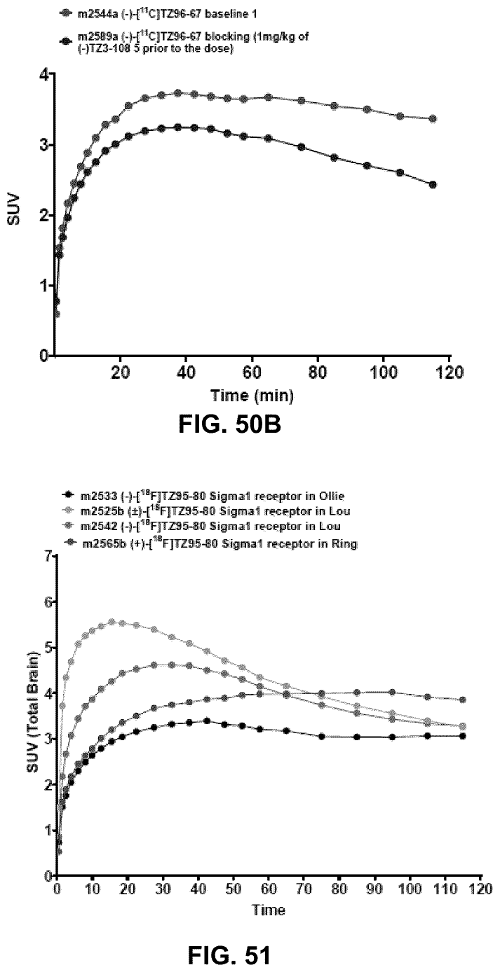

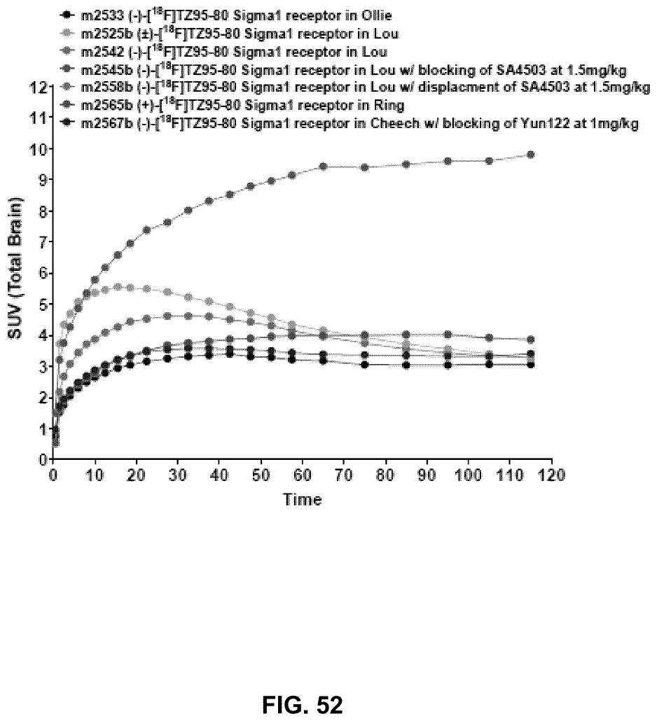

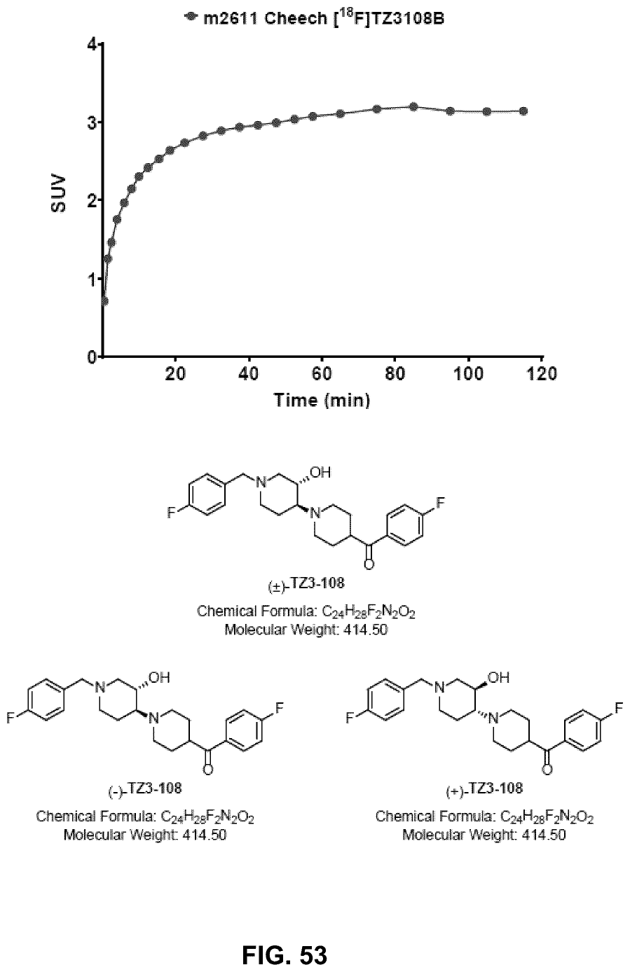

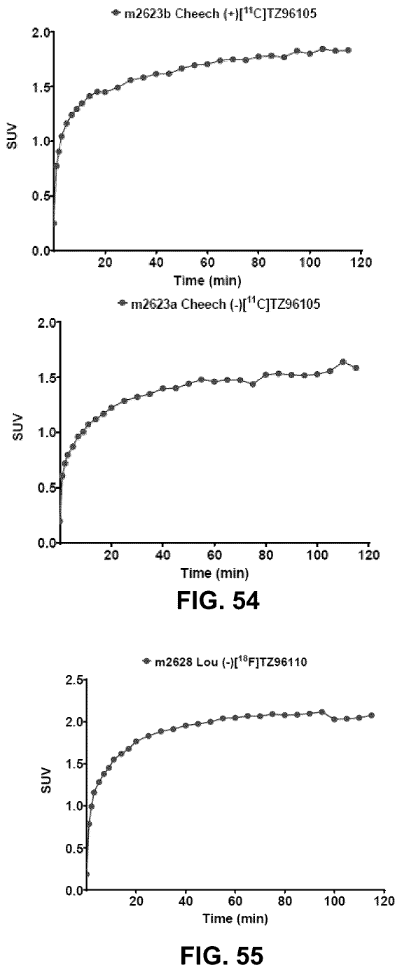

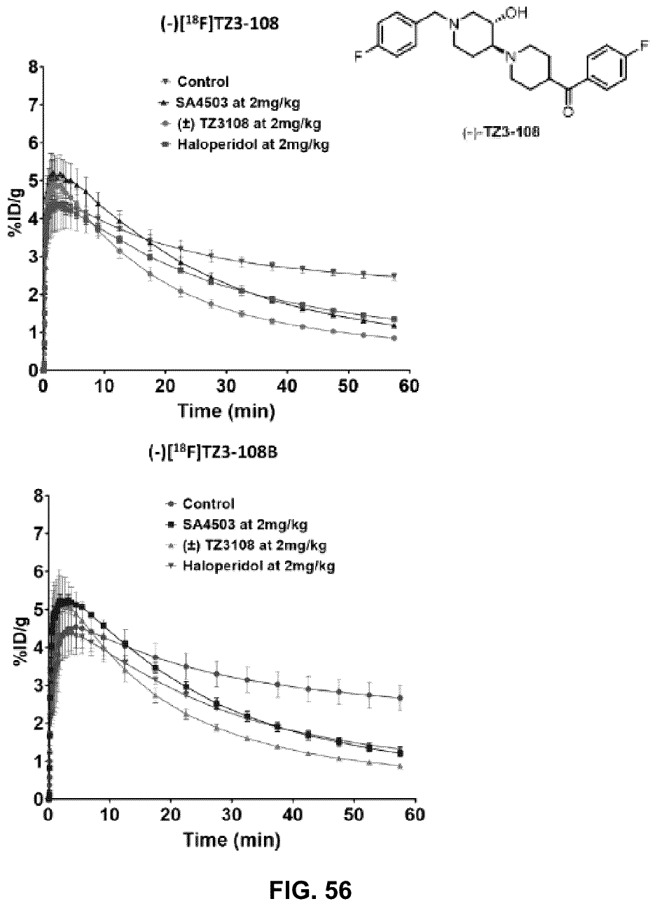

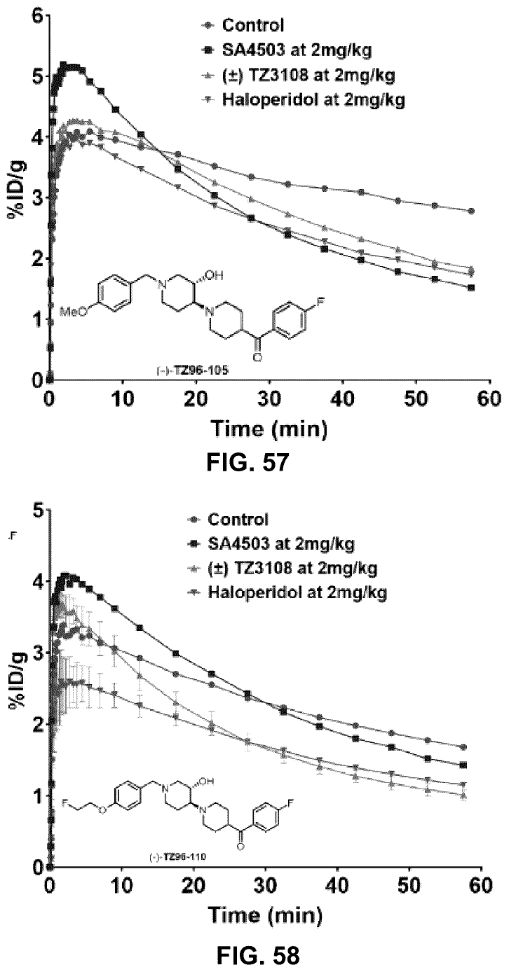

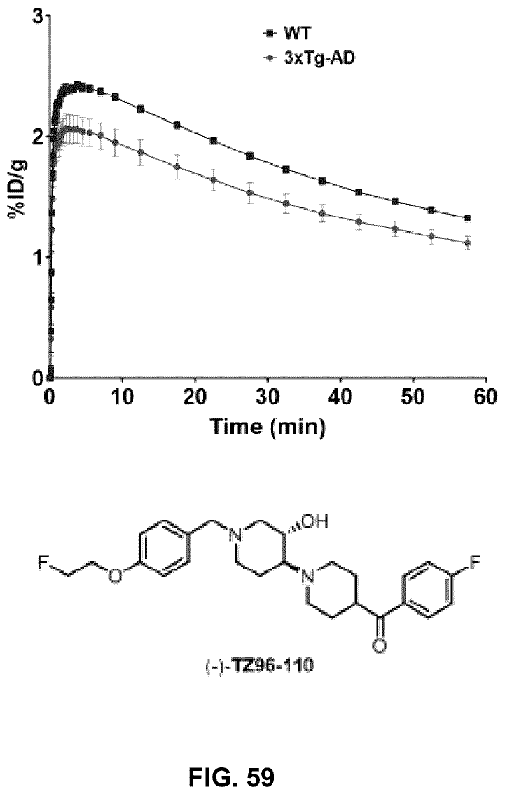

Results: Out of Six, five new σ1R ligands (±)-1, (−)-1, (+)-1, (−)-2, and (−)-3 were synthesized successfully. The in vitro binding assay indicated all six compounds were potent to σ1R with Ki values of 14.1±1.7, 2.7±0.9, 14.3±2.8, 13.8±1.5 nM for (±)-1, (−)-1, (+)-1, (−)-2 respectively, and 1.8±0.4 nM for (−)-3 also called as (−)-TZ3108. All compounds were selective to σ1R over σ2R and VAChT with a 27.3, 150.1, 40.1, 44.7 and 3866.7 fold selection over σ2R and 27.9, 120.4, 57.0, 49.7, and 544.4 fold selection over VAChT for (±)-1, (−)-1, (+)-1, (−)-2, and (−)-3 respectively. The F-18 radiosynthesis of (±)-[18F]1, (−)-[18F]1, (+)-[18F]1 (−)-[18F]2, and (−)-[18F]3 was achieved with good radiochemical yields up to 21-30%, high chemical and radiochemical purity (>95%), and high molar activities (>42 GBq/mmol, decay corrected to EOS). (−)-[18F]3 was made by introducing F-18 from two different positions and methods, it is one molecule for two radiotracers (−)-[18F]3 and (−)-[18F]TZ3108. PET imaging revealed that all six radiotracers entered the monkey brain well with good brain uptakes. (−)-[18F]1 showed initial brain uptake with an SUV of ˜2.9 at 30 minutes, followed by gradual washout, indicating it is the most favorable σ1R radiotracer for brain imaging among the six tested F-18 σ1R tracers. Further PET brain imaging studies in mice with σ1R blocking agents, (±)-TZ3108 and Haloperidol at 1.0 mg/kg showed a significant reduction of brain uptake, confirmed the in vivo σ1R binding specificity of (−)-[18F]1. Biodistribution study in SD rats indicated (−)-[18F]1 had a good initial brain uptake up (ID/gram) of 1.0 at 5 min, followed by gradual washout with the brain uptake of 0.34, 0.30, and 0.25 at 30, 60, 120 min, respectively. The radiometabolism data revealed that (−)-[18F]1 was stable in NHP plasma in vivo with parental percentage (%) of 98, 91, 85, 77, and 75 at 5, 15, 30, 60, and 90 min post-injection, respectively. Moreover, 60 min dynamic PET imaging of (−)-[18F]1 showed a significantly lower brain in 3xTg-AD mouse model of Alzheimer compared to age-matched wild-type mice with a 24.1% decrease in average SUV from 10 to 20 min (FIG. 48A, FIG. 48B, FIG. 48C).

Conclusions: We successfully synthesized five new F-18 labeled σ1R radioligands: (±)-[18F]1, (−)-[18F]1, (+)-[18F]1, (−)-[18F]2, and (−)-[18F]3 with good quality for animal study. PET studies in cynomolgus macaque indicated these radiotracers have excellent blood-brain barrier permeability and brain uptake. Radiotracer (−)-[18F]1 showed the most favorable tracer brain washout pharmacokinetics. Both PET imaging and biodistribution studies of (−)-[18F]1 in rodents pretreated with σ1R ligands showed significant brain update reduction, suggesting it has in vivo binding specificity for σ1R, and radiometabolite analysis in NHP confirmed (−)-[18F]1 has good metabolic profiles. PET with (−)-[18F]1 detected σ1R decreased in AD mouse model compared to controls. Our data indicate (−)-[18F]1 has a great potential for quantifying σ1R in CNS particularly for the σ1R expression change in AD. Further characterization and validation are warranted prior to transfer into clinical validation in patients.

What is claimed is:

1. A composition configured to target a sigma-1 receptor, the composition comprising a compound selected from:

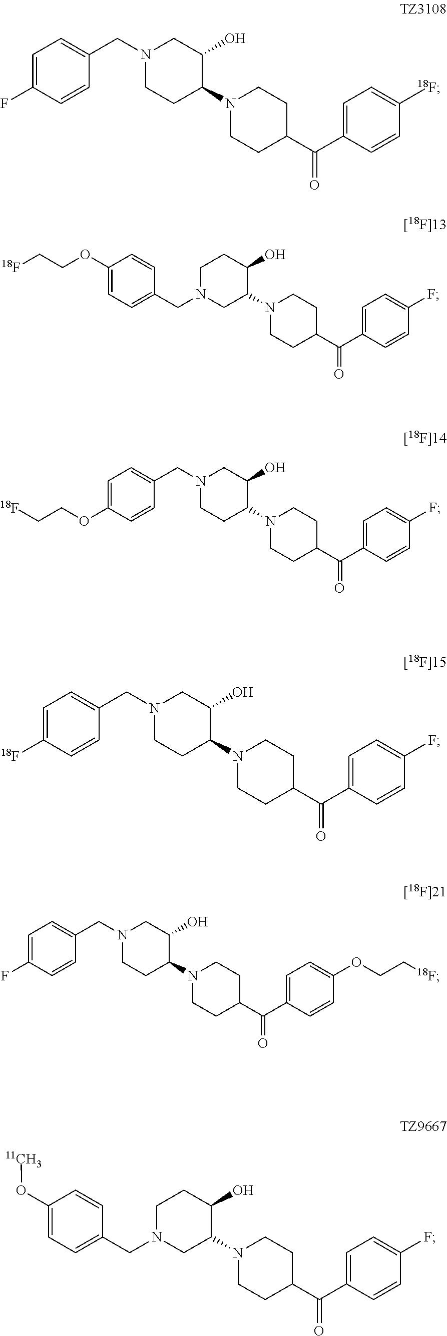

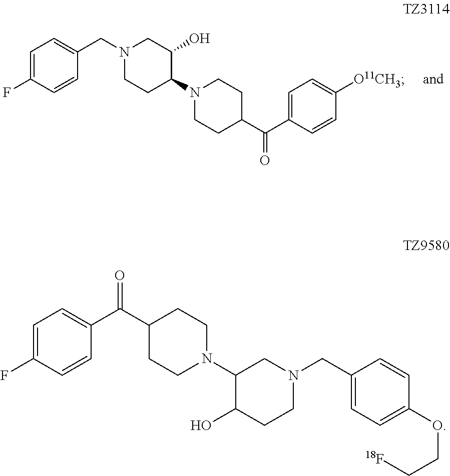

2. The compound of claim 1, wherein the compound is radiolabeled with a radionuclide.

3. The compound of claim 2 wherein the radionuclide is a radiohalogen.

4. The compound of claim 3, wherein the radiohalogen is F-18.

5. The compound of claim 2, wherein the radionuclide is C-11.

6. The compound of claim 1, wherein the compound is [¹⁸F]13.

7. A method to assess treatment efficacy of a sigma-1 modulator in a subject, the method comprising:
a. administering a therapeutically effective amount of a radiolabeled compound configured to target a sigma-1 receptor of the subject;
b. acquiring at least two radioactive images of the subject, wherein a first radioactive image is acquired before a sigma-1 modulator treatment begins and a second radioactive image is acquired after the sigma-1 modulator treatment begins;
c. characterizing a sigma-1 expression of the subject based on the acquired image; and
d. assessing a treatment efficacy of a sigma-1 modulator in the subject based on the characterized sigma-1 expression.

8. The method of claim 7, wherein the radiolabeled compound is configured for uptake to a brain of the subject, specificity for the sigma-1 receptor, and fast metabolic kinetics.

9. The method of claim 8, wherein the fast metabolic kinetics comprise clearance of the radiolabeled compound from the brain within 60 minutes.

10. The method of claim 8, wherein the radiolabeled compound is a compound selected from:

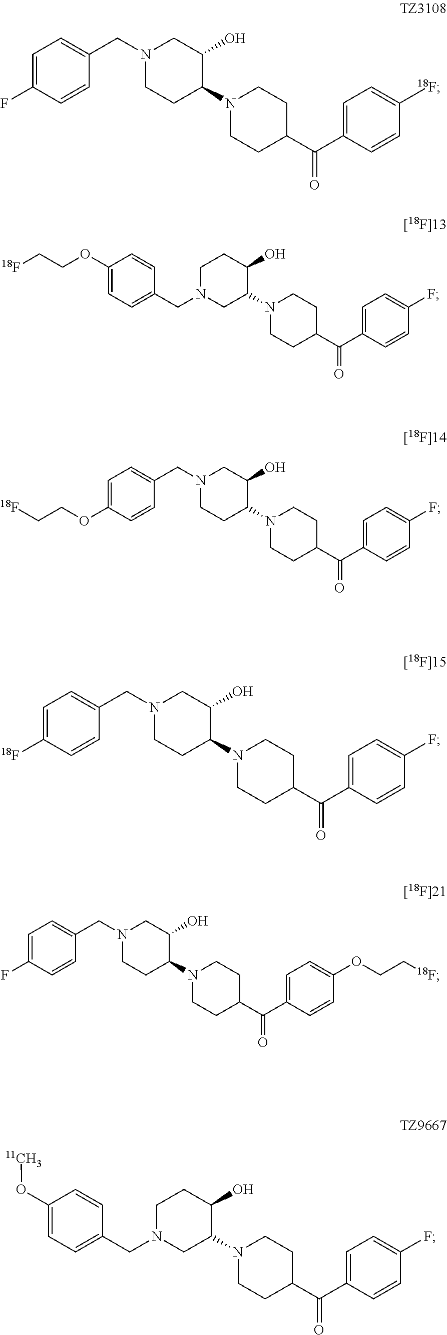

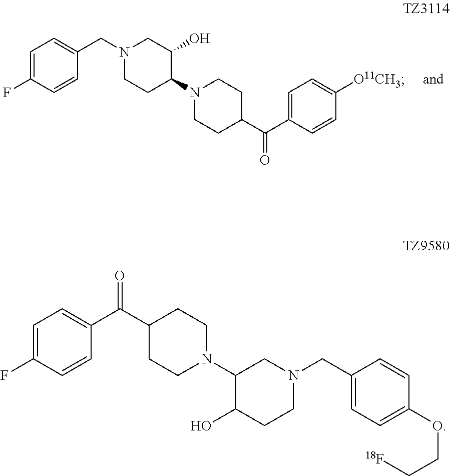

11. The method of claim 10, wherein the radiolabeled compound selected is [¹⁸F]13.

12. The method of claim 7, wherein the at least one radioactive image is selected from PET and SPECT.

13. The method of claim 7, wherein the radiolabeled compound is administered at a dosage ranging from about 7 MBq to about 370 MBq.

14. The method of claim 7, wherein the subject has a neurological disorder.

15. The method of claim 14, wherein the neurological disorder is Alzheimer's disease.

16. The method of claim 7, wherein assessing the treatment efficacy of the sigma-1 modulator in the subject based on the characterized sigma-1 expression further comprises comparing the characterized expression of sigma-1 receptor in the first radioactive image to the characterized expression of sigma-1 receptor in the second radioactive image, wherein:
the treatment is characterized as effective if the characterized expression of sigma-1 receptor in the second radioactive images is the same or increased compared to the characterized expression of sigma-1 receptor in the first radioactive image; and
the treatment is characterized as ineffective if the characterized expression of sigma-1 receptor in the second radioactive images is the decreased compared to the characterized expression of sigma-1 receptor in the first radioactive image.
